SEQUENCE LISTING (23 sequences)

SEQ ID NO: 1 - DNA, 9142 bp, Artificial Sequence
  Description of Artificial Sequence Synthetic polynucleotide
tgggagtcaataaacccgatgtgcgttggatttgccactaccagccgcccctgcaactcagtgaatatctccaagaggtgggacgcgctgggcgagatggcgaagcggcacaggccctggttttggtgagcgatcgctggggcttggatcgcgaagatcaacagcgttggtctttttttcagcaccaaagtcaagacacctacaatcgcgccatggcacttcagacgcagctgcccctccagggtaatctgcagcaactgcggcaacactttcctgaagtggaattgaccctggcattactgcatcaacagggggccctccgctggcaagatccctttcactattgccgtcaacccttggcacaggtgccacccccacccaaagaccctcaagaacagttgatgcaaaagttcctctatcaccggggctgccgctggcagtttctcctccaagcctttggttttgccactgaggcaaggggattccactgtggccattgcgatcgctgtcggccgccgcaccgctcccgcaaaataccgtaaattgccagcgctgtatcactggaatattgggtacactggcacatagaacggtcgctttaccattggtaggcaaaagtttctcagcagtcattctgttgccgcaaggtaggggttgcaggcatggggctactacaagttgaggaaattcgcgaagcacttcaagatgtgctttcagaacacgcccttgttgtgcaagttaatcagtttcgcaaccaattaaacattattttgaacaagccccccggcaccgttgcccattattctgccctagcggattttctcaagtcgcgcttgggacagtttcatctcaatgatattgaccgcattaaaataattggccgcatacagggttcgcctaaacccgattgggaagaggtcattgatctacgtccccccaacccagccctagctgcccctgtgtatgcttcttctgccccgtgggtggtggcgatcgctgctggctttgtcagtttactggtgatctttagctatcaccttggtcagtagcagcaacagcaacggctgtagccgttgatcgaaggttcctttggtcaaaagggcgtcgtgatgacggactttaagtggcacattgagggtggtacagggtttattgtcggggttcttaaaaactacagtaaagggtattttcgcttagttcaggcggactttgaactctttgaccaaggcggtcagcaagttgggacagtggcggtacaggtttatggtcttggccctgaggaaacatggcaattccgtgaactgatagccaatcatcaggcagtgcgagcacggctggtaaaattacagtcattcaattaaggtttttctaatgtttaggtttccccagcagggagcgacaccgcttgctatggcacaccttaaagccctgatctttgatgtcgatggcaccttagcagatacggagcgggatggccatcgtatcgccttcaacaaggcctttgccgccgctggtctagattgggaatgggacattcccctctatggtcaactcctggcggtggctgggggcaaggagcggatccggtattaccttgagtgctttcgtcccgattggccacgtccccaaaatttggatgctctgattgccgatttacacaaggccaagacccgctattataccgagctattggcggcaggggctattcccctgcggccgggggtgaaacggctcctcactgaagcccgggaagcaggattacgtttggcgatcgccaccacgaccacccctgccaatgtcaccgcactccttgaaaatgccctcgctcctgatggcgtcagttggtttgagataattgctgccggggatgtagttccagccaagaaacccgcgcccgacatttacttctacacgcttgaaaagatgcgcctctcaccccaagagtgccttgcctttgaggattccgccaatgggattcaggcggccactgccagtcacctagcgaccattatcacgattaccgactacaccaaggatcatgattttcgtgatgcagcgctggtcttggattgcttaggggaaccggactacccctttcaggttctgcgcggtgaggtgggttggacaacctatgtggatgtccccctattgcgatcgctgcaccagcagtggacaagcacgttgagtcagggataattttctggccgcagcgttttacattgaatatgacccccttagtctgaggatcaaggaacataatgtacacgattgatttaattctgcgtcatgtccccatgcccgtcagcattgaacgcaaggaaagtgcagcagcgatggcagtctatcagcaaattcagcaggccatggccagtggtactccaactttcctcgaactgacgtgcgatcgccaagtgggcaagaagttaacggtgctcacctcagaaattgtcgccgtgcaaatggcggataaggatgccccctccagtactatcagtcgtgggggattctttgctcaattagtgcagcaaaccagcaactgagggaaaatgcctcaataaagttgagtttttcttggcaatgctgattctttgccgttaggatactaagcagaccgatccgtaggggaacgtgaagcaaatcctccccgtctgaaagtcaggtatctctggtgtgtcgtaatagggttgtctatggtgcagcgtttcctgccggttctgattttgttggggtgtagttttggtcttgcgacccctgcccttgtgcgtgcccaagccaatcagggctttacgtttacttggggtgaggggccgagtggccgacagcagttgcaataccacttagataacggcacccccggttttatgggcgatcgctattggctgcggctgggtcagcagaaagtggccatcaatcgcattaacattacctatcccgactactacaacggtattattgatcccaaaggcattgaggtgcgcatcggtggcgatcgcggcaatcgcttcttccaatttcgccgtgaccccggcaccaaaattcaattggcggaagtctccgttgatcgcgataaccgcgtgattgatattgtgccggctgaggtgattcccgccggaacaccggtgcaagttattctcaataatgtgcgcaaccctaacaatggcggcatgtactatttcaatgcccgcattggctcccctggagatattcccctcatgcgctacgttggcacctggattctcagcattgccaataactaaaacccgtcaaactcgagcattggtgagcgggttagccatttctaactattgcggggcgatcgccctagactagttttttgtctattattgccggttcactctttacaccagatgccagattccgttaggtcttcattcccctccatttctcctctgctcacgcctctgatgtaccgcctcgtgggggacgttgtcctgcggcgctattttcgtacccttgaggtgcaagggcaggagcgggtgccccaaaggggtccagtgatcttggcccccacccaccgttcccgctgggatgcgctgattattccctatgtcactgggcggcgggtgagtgggcgcgacctctactacatggtgtcccacgatgagatgttgggactacagggctgggtgattgctcagtgtggcggttttcccgtcaatacccaagcgccttcggtgagtgcgttgcgtacgggtgtggaactgctccggcaggggcaagccttggtggtgttccctgaggggaatatctttcgcgatcgccagattcatcccctcaagccggggttggctcgcttagcccttcaggcggcccagcgctgtgaacaagcaatccagattctgccaattttactcgattatgcccagccctacccacagtggggaagtgcggtcaaggtaatcattggggctcccttgagtaccgacaattacgatgccagccggccaaaaagtgctgcccaacaactgaccagtgatctctttagaagacttcagcagctccaaggggggcgatcgcccctgtgttttgcttagacctcaaacttccatccccgcggccgcaaaaaaaacgggccggcgtattatcgccggcccgagtaacaccgtgcgtgttgactattttacctctggcggtgataatggttgcaggatccttttgctggaggaaaaccatatgaaaggaccaataataatgactagagaagaaagaatgaagattgttcatgaaattaaggaacgaatattggataaatatggggatgatgttaaggcaattggtgtttatggctctcttggtcgtcagactgatgggccctattcggatattgagatgatgtgtgttctgtcaacagagggagtagagttcagctatgaatggacaaccggtgagtggaaggcggaagtgaatttttatagcgaagagattctactagattatgcatctcgggtggaaccggattggccgcttacacatggtcgatttttctctattttgccgatttatgatccaggtggatactttgagaaagtgtaccaaactgctaaatcggtagaagcccaaaagttccacgatgcgatctgtgcccttatcgtagaagagctgtttgaatatgcaggcaaatggcgtaatattcgtgtgcaaggaccgacaacatttctaccatccttgactgtacaggtggcaatggcaggtgccatgttgattggtctgcatcatcgcatctgttatacgacgagcgcttcggtcttaactgaagcagttaagcaaccagatcttcctccaggttatgtccaactgtgccagctcgtaatgtctggtcaactttccgaccctgagaaacttctggaatcgctagagaatttctggaatggggttcaggagtgggcggaacgacacggatatatagtggatgtgtcaaaacgcataccattttgatgtctaacccccttccttgcccacagcttcgtcgatggcgcgaaatttcgggtaaatataatgaccctcttgataacccaagagggcattttttaggcgcgccctaagcgtccgtaggcacaattaaggcttcaaattgttggcgaagctgctcagtcacttccttgacggcttgccgtgccccttggcgatcgcgccggtacagaggccaatagctctctaaattgagagggtcgccgacactgaggcgcacctgccgcaaacccaccaaacgattgagattcgagctttttccctctagccaatcaaatgtgcgccagagaatcagcgcgacatctgcaaagcgatgaatcgtgaatttctcacggatatagctacccgtaattgaggtaaatcgctccgcaagacgcatatgacgcaatcgcacattggcttcctcggccaaccaatcggctaggcagcgctctacggccgaaagttgtgccaaatcactgcgaaacatccgttcccaagcagcctgttcaatgcgtcggcagcgactcacaaaatcggcactgggcttcagaccaaagtaggactctgccaccacaagggcgctgttgaggaggcgctgaattcgcgctgccaatttagcattggcagagtcaaaggggggcagttcgggaaaatcttgaccataggaggtggcataaaaagcctccaggcgatccaagaggtggatcgctaaattcagcaggcggcggtagaggtcgtctggctgggtactgtgagaatctgtagggcacccaaggcggttctccagttgtgccatcagccttgccatgcgctcccaagagggctgactgaggctgtactgaatgccaatgggaagaatgaccacggggagcgatcgccccgccttggctaaatcttctagacaccaaaatcccagttgggccacccccggctccaaaggtgcgaccagttcgttgtgctcattcgttgctccctccggcgctgccgctaggggaaatcgtcctccgagaagtagctcccgcgctgagcgcagggcttggctatcgagcttaccgcgcatgatggaaatcccccccaaccgtgaaaagagccaaccaatctgcgcccctgcccagaggggaatcccgcgatcgtagagaaaatagccatttgtcggcggacgcaagggaatgcccagccgccgtgctgtttgcggcagtaaatgccacatcaaatagcccatcaccaacggatcatccgtacagggatggcgaaaggcaatgaggagccggacctgtccctgctgaaactgctggtaataacgggcaagggtctccacattcaccccttcaacccgctgtagcccaagaccatagcgaatgtagaggggcaggagtcttgctactgtccaccagacggggtagctaaaccgctgggggagaaaatgcaacggcggttgggcagttgtcactacactggacattaggcaagctcctcagggcaatggctaaactgaggcagtggccaactccgcaattaactgctctaacatcggttgatcggcccaatagacagcattacaaaactgacaggtggcttctgcctttgcctctgtggctaggatatctcttaattctgcctcccctaggagcttgagtgccgctaacatccgttcatgggaacagccacagtggaagcgcaccatttgccgttggggcaagatttgtaaatccatatcccctaagagttcctgaaagatatctggcagtgtccgccctgcctgtagcagtggtgtaaagcccttaagattggccacccgttgttcaagggtcgcgatcaggtgttcatcattggccgctttgggtagcacctgtaacatcaacccaccggcggcagtcaccccggactcttcgacaaaaacacccaacatcagggcggagggggtttgctctgaggtggcgaggtagtaggtgatgtcttctgcaatttcgccggagactagctccaccgtgctggaataggggtagccgtagccaagatcgtggatgacgtagagatatccctgatggcccaccgctgcccccacatcgagtttgcccttggcattggggggcagttcaacactggggtactgcacatagccgcgaactgtgccatcggcaccagcatcggcaaaaatggttcctaggggaccgttgccctgaatgcgcacattcacccgtgcttggggctgtttgaaactggaggcaaggattaagcctgcggccatggttcgtcccaaggccgctgtggccacgtaggacagttggtgacgtttgcgggcttcatcagtgagttgagtggtaatcacacctacggcccggatgccttcggcagcggcagttgctcgcaacagaaaatcggccatgttcaacctacgaaatgttttgttacatttagtgtgacatactcccaccgctgaccagggcacaatggggcaaaaaaccatcaatcctgcctttggtgaccgatccagtacagccagccagggcttaagactgggaagacccctagcactggggctagaaaattggcgatgataggcaagcaatagtcattcagcgtccagtcattccgcctatggccatgcccctcactgtcttgcctgccacaactgttttgacagaagcgactcaattgccccagggcggcttgattacggagattccgacgctggcgatcgcccaccgtttggcccagcagttgcgccgccattggcccctagagacccccttaacgctgattgatgcgcaataccagagtatccccctgacccttggggaattggccgagctcaccgatgccaactgtcctttacagctctatgtgccgccccccttgccagaggccttgacgcaatttcaacgcctgatggatgtggttcgagagctgcgccatccggagcgtggctgtccttgggatttgcagcaaaccccaaccagtctcattccctatgtccttgaggaagcctatgaagtggtacatgccctgcaggagggagatgcgggggcgatcgccgaagaattgggagacctgttgcttcaagttgttctccagagccaacttgcccaagaagccggccaatttacccttgctcaagtcattcaaaggattaccgataaactcatccgccgccatccccacgtctttggtgaagtggcactcaccactgctcaagaggtgcgcgaccaatgggagcaaatcaaagcggctgaaaaaggcaccgaactccccctgagtcaaacgctgcaacgttacgcacgcaccctcccacccctgatggccggcatgaaaattggtgagcgagccagtcgcgctggcctcgattggccgacgattagtggtgcatgggagaaattttacgaggaactggcggagtttcaggaggcccttctgcaagggaatgctgagcaacaggcagcggaattaggagacctgctcttcagtgtgattaaccttgcccgctggtgccaactggatcctgttaatgccctgcaacaaacctaccaacgctttattcaacgcttggcctgtattgaggcagtcatcgatcgcccccttgagacgtacaccctagaagaactagaagccctctggcaacaggccaaagtacagttagccaccgacagcgaggcaacccctatggagactgaggaagaggcctagtccgctgcggcccttgccaccttcagttcatcgagattccacagggggccccccagcgccgtgggcttggcgccaatgacatgattgcgaaaagctgtaagggagaggggattcacgaggtaaataaaggggagatattcctgagctagtcgttgggcttccgcataaatttgctgccgtcgttccagattgagctcctgggcaccttggacatacaggtcactgatgcgctgctcccagtcagcgacgactcgacccgtaatgggtggttgattcggtgacggttgctgattgaatgtatgcaaaaggccatccacacgccagatattggcaccgctattgggttcattgccccccccagtaaagccgaggatatgggcttcccactctagggaattggagagacgatccacgagggtaccaaaggccaaaaattgcagatccacctgcatgccgatcgcccctaggtcctgctgaacttgcgtcg

SEQ ID NO: 2 - DNA, 9618 bp, Artificial Sequence
  Description of Artificial Sequence Synthetic polynucleotide
tccgcgggaggtgtaatgccgatggcccccttgcggaaaacctatgttctcaagctatacgttgccggtaacacacccaactcggtgcgtgccctaaaaactctcaataacattcttgaaaaagaatttaagggagtctatgcactcaaagtaatcgatgtcctcaaaaatccgcaactggctgaggaagataaaattttggccacgcctacccttgccaaagtcctaccgccccctgtgcgccggattattggggacttgtcgaatcgtgagaaggtgctcattggcttagatctcttgtatgaagagattggtgaccaagccgaggatgacttaggcttggaataggcacagtccttagagactctcagtttagaatagcttcttggaatttttgcgcaataccgaatctaaaaatcttctatgacaaacctaccggaacatcagtctagtccaacggagcagtcctctgcggaagtcaagaaaatcccgacgatgattgagggctttgacgatatcagtcatgggggacttccccaaggacgcaccaccttagtcagcggcacttcaggcacagggaagaccctttttgcagttcagtttctctacaatggcattaccatttttaatgagccaggtatatttgttacatttgaagaatccccccaagatattatcaaaaacgccctcagttttggctggaacctgcaaagtctgattgatcaaggcaagctatttatcctggatgcttctccggatcccgatggccaagaggtggctggtgactttgacttatctgctctgattgagcgcattcagtatgccattcgcaaatacaaagcaacccgggtctccattgattcggtcacagcagtgttccagcaatacgatgcggcctccgtggtgcggcgggaaatttttcgcttggcttttcgcctcaagcaactgggcgtgaccacgattatgaccactgagcgggtagatgaatacggccctgtggcgcgttttggtgttgaggagtttgtctccgacaatgtggtcattttgcggaatgttctcgagggagaaaggcggcggcgcacggtcgaaattctcaagctgcggggcaccacccacatgaagggggaatatccctttacgatcaacaatggtattaacatcttcccgttgggggccatgcgcttgactcagcgctcatcgaatgtgcgggtgtcttcaggggtcaagaccctcgacgagatgtgtggcggtggcttcttcaaggattcaattattttggccacgggcgctacgggtactggcaagacgctcttggtcagtaaattcttggagacgggctgccaacagggagaacgagccctgctgtttgcctatgaagaatcgcgggcgcagttgtcgcgcaatgcctcctcttggggtattgattttgaggagttagaacggcgcggtttgttgcggattatttgtgcctatccagagtcagcggggcttgaggatcacctgcaaattatcaagtcggagattgcggactttaagccctcacgggtggcgattgactctttgtctgcgttggcgcggggggtgagtaacaatgccttccggcagtttgtaatcggggttactggatttgccaaacaggaggaaatcactggctttttcaccaacacgacggatcagtttatggggtccaactcgattaccgagtcccatatctccacaattacagacaccattttgctgttgcagtacgtggaaatccgcggtgagatgtcgcgggcaattaatgtctttaagatgcgtggctcttggcacgacaaggggattcgggagtatgtgatcactgagaagggggcagaaatccgcgattccttccgcaactttgaggggattattagcggtacccccacccgcatttccgtggacgaaaaaacagagctggcgcgaattgccaaggggatgcaggatctagagagcgagtagccccatgcagttaaaccaagttattgtggtgcacaaggcgggcgatcgccagagcaaggaatgggcagatcgtgcctcccgtcaactacaacagcgtggcgccaatgtgctggtagggcctagtgggcctaaggacaacccttaccccgtctttatggcctctgtgacagagccgattgatctcgccgttgttctggggggcgatggcacctccttagcagcggcacgccatctcgcagcggctggggttccaattttagcggtgaatgtgggggggcatttggggtttttgacggagcccttggagttgtttcgcgatatggaggcggtttgggatcgcctggagcgggatgagtacgcgatgcaacagcggatgatgctgcaagcccaggtttttgaagggtcaaaggctcatccggaagcggtgggcgatcgctactatgccctgaatgaaatgtgcattaagccggcctctgctgatcgcatgatcaccgccatcctcgagatggaaattgatggcgatgttgtggatcagtaccaaggggatgggttgctggtggccacgcccactggctctacttgctatacggtcgccgccaatggccccattttgcatccagggatggaagccctggtggtgacacccatttgtcctttgagtctctctagccgccccattgtcttgcctgcgcgctcctcagtcagcatttggcccttggaggatcacagtctcaataccaagctgtggatggatggtgtcctggccacctccatttggccaggacagcgggtacaggtgacaatggccgattgtcaagctcgctttatcatcctgcgggatcactactccttttatcaaaccctacgggagaagttagcctgggcaggggcacggattccctatcacaacaatcaccgcaattagatcacaaccgcccctccagaaggtctttataattggggcattcctcactaaacccttgctatgattctcagtccctttgaacgcgccgttcttggccaagaggcggaagccctggttgatcagttgttagaaattgggatttccctctctgccagtcaatccctagaggaattgctgcatctgattctcacgaaaagtcgccaaatcactgctagcgatgctggcacgatttttctagttcagcgggaacgggcagtgctggaattcaaggcagctcaaaacgatagcgtcacccttcctgagcaagtgcaggactataccatacccctcaccgccgatagcttggtgggctatgccgctctcacgggggaatccctaaatattgccgatgtgtatgccctcaaggggagcgagatgtaccagttcaatcgctcttttgatgaagccctccactatcgaacctgttcggtgctggtggtgccgatgcaaaatattagcggtgaggtgattggcgttctgcaactgattaaccgcaagcgatcgcccgatacccggctgagaccagaaaccagtgtggccctcacccagccctatagtccttgggaagaacatattgtgcgatcgctggccagccaagcggcggtgattattgagcgcaatcatctgctcgagagtattgaacagctctttgagggatttattaccgcttcagttcaagccattgagacgcgagatccagtcaccgcagggcattcggaacgggtggcagcgctgacggtgcgccttgctgagatcaccaatgccacctctaggggagtctttcgcgatgttttctttagcgatcgccagctccaggaaatccgctatgctgctctgctccacgattttggcaaggtgggcgtgccggaggcaattctcaacaagcaaaagaaattctaccccgaacagctagaggtgattcgccagcgctttgccctcgtccgccgcacccttgaaatggaaacggctcaagccaaagtcaattatttactctcccatccccatcagccccataccccacaacagcggtgtcagtcctgtacttttttacgagacctcgatcagcaactccagcaacaactgcacaccctagaggcctactggcagctaattgagcaggccaatgagccgcaaattcttgaggaggaacccctggctcagcttcaggaattgacccagttttattaccgcggcactgatggggaactccatcccctgatcacggccagcgaactggagcaactcttggtgcggcggggcaatctcacccaaggggagcggcgcatgattgaagcccacgtcacctatacctacgagtttctctcgcgcattccttggacaccccacctgaagaatgtgccgatcattgcctatggtcaccatgagcgcttaaatggcagtggctacccccgcggtattggtgccgccgaaattcccctacaaacccaaatgctggcgatcgcggatatttacgatgccctgaccgccaaggatcgcccctacaaaaagagcctacctgtggatagggccctagggattttgtggcaggaggctagggaatttaagattaatcctgatctggtggaactctttgagcagcaggaggtctttcgggtgctggggcaccagcgctaggcggccgcaaaaaaaacgggccggcgtattatcgccggcccgagtaacaccgtgcgtgttgactattttacctctggcggtgataatggttgcaggatccttttgctggaggaaaaccatatgaaaggaccaataataatgactagagaagaaagaatgaagattgttcatgaaattaaggaacgaatattggataaatatggggatgatgttaaggcaattggtgtttatggctctcttggtcgtcagactgatgggccctattcggatattgagatgatgtgtgttctgtcaacagagggagtagagttcagctatgaatggacaaccggtgagtggaaggcggaagtgaatttttatagcgaagagattctactagattatgcatctcgggtggaaccggattggccgcttacacatggtcgatttttctctattttgccgatttatgatccaggtggatactttgagaaagtgtaccaaactgctaaatcggtagaagcccaaaagttccacgatgcgatctgtgcccttatcgtagaagagctgtttgaatatgcaggcaaatggcgtaatattcgtgtgcaaggaccgacaacatttctaccatccttgactgtacaggtggcaatggcaggtgccatgttgattggtctgcatcatcgcatctgttatacgacgagcgcttcggtcttaactgaagcagttaagcaaccagatcttcctccaggttatgtccaactgtgccagctcgtaatgtctggtcaactttccgaccctgagaaacttctggaatcgctagagaatttctggaatggggttcaggagtgggcggaacgacacggatatatagtggatgtgtcaaaacgcataccattttgatgtctaacccccttccttgcccacagcttcgtcgatggcgcgaaatttcgggtaaatataatgaccctcttgataacccaagagggcattttttaggcgcgccctagggtggatcggcggacgattgcaaaaacgagagtttccacagcgtagctgccagccaattggtacaggtatgggcaacgatcgctaagagtaaattattcgttgccacagcactataggcaaagaatccgcccacaaaggtagcccacagggcatagggccactgctgccgcgatccagcgtgcaaaatgccaaagcacgcagaactgccaataatccctgcccagttgagccccaaactcggtaggagcaccccgcgaaagagcagctcttcactaaggccgggcagaatgccaatccaaaatagatcaggccacagcagtggtgaaagcacaagtttcaggtaggtatctgaggcgtggcggtaggccggccagaggcgatacaaaatggcgccaatgccggtaattcctaggcagagggcaatgcctaaaaccactgcccagacatcccagcgcagcggcagcagtcccccagaaaagggggtaaataaccacacccgcgccaaaatcagccacaggatggccgttaacgccatggccactaagacctgtgtacgactcagaggctcatcgggtaggggggactcctccataggtctacgctttctggaactgaccaaattggaagttatagacctcctcctctttttcagagatcaatttcaaatctgagcaagggcgggccacacagaggaggacatagcctttttcccgcagttcgggactcagccccattgcatctccgtgatccacggtaccctcctgaatttgggccgcacaggtggtacataccccggcattgcaggaactcggaagatcaattccggcagcggtggccgatcgcaggaggggtttatcggcactggcttcaaaagtgtaggtttgtccttggtgcagaatctcaacacgaaaggtttgggtcattctggcagtgagctatgacgcaacatcttccctattatccccctaatcctcgcgatcgctggcttcctcgggggcagacttcaaccatgccggcaaaggatcaggaatcggcacacgctggcggtggggcagttgcaggcacatgtgttgcgtctgggcaatggctacccgatcccccccttcgttgtagagagtataggtcagttgaaaacggctagtatccagtctttgggggtcaatggtcacccgcaggcgatcgccacagtagaggggtttcaaaaaccgtatctgcgcctccgtaatcggcacaatgaggccactgttgctgaaaaattgccgcagatctacccccaattgggcaagggcatcctcataggcctcatggcaaaaccgcagcagattggcaaagtagactaccccagccgcatcggtatcggcaaaatgaactgtgcgctgatagtcgcgcaggggtgttggattcatctatcgtccttccattgccatcccatagggttgtccaacacaagccatgggcaaaaacgcgccacagcatttgttgttaatataggatacagctcttttgcaaccaattcccatccctaaaccgatgagtaacaaaggcagttctgatctgcgacttcttttaagcacgctggtgatcagtggcttagtcgcaggactggcctattggcaactcagtcaacactggacccgctcccccgatcaaaacgctggctcccccctccacaccccaacctcaaagtggcaaaaaattgccctcgcgatgaccctgcggggccatgaagatgaggtgaacgcgatcgccctgagtcccgatggcaatttcctcgtcagtgctggcgacgatcgcaggctgtacttctggaacttggctacgggaactgccctaggacaagccaaaggtcacaccgactggatctatgccctggtgatgactcccgatggtcagacggtgattagcggcagtaaagacaaaaccatcaaactatggggggtgggcgatcgccaactccaagccaccctcagtggccaccaagattttgtgaatggcttagccctcagtcccgacggtcgcacccttgccagtgccagctatgatcacaccgtcaaactgtggaatgttcccagccgtcaggaaattactacgctcaaagcaaatgagggcatcatgctcagcgtcgccattagtcgagatgggcgttttttagccacgggtggcgtggataaactcatccgcatttgggatttgccctcccgccgactcctgcgcaccctggaaggacacaccagtgatgtcaatagcctcgccttcacccccgacagcagccaactggtcagtggcagtgacaaagatggtataaaactttggaacctgaccacaggagaactgcagcaacagtttggcactgagggcgggcaggtctttagtgtggcagtgagtcccgacggcagcacccttgccagtggtcacggcgatcaaactgtcaaactttggtccctctctggtcagttattgcggaacctcaagggacactctggcgctgtctacagtgtcgtctttggtcaggatcaactgatctccgccagtgaagacaaaaccatcaaagtgtggcgtctttttcccgaaaccccatagagaactcgcgggcctcacctacggcacaaaaaacggctaagatccccaagaatcttagccactgagaacaacggctggaatttttttagcccacacttccctctagcttcaggctcagcaggcgatcggcctcgactgcaaattccatcggcaattgattaaagacatcgcgacagaagccactaatcatcattgagacggcatcttcagcggaaattccccgctgggcaaagtagaagagttgatcttcaccaattttcgatgtcgaagcctcatgctccacctgggcagtggggttttgcacctgaatataggggaaggtattggcagcggccgtatccccaatgagcatcgaatcgcattgggagtagttgcgtgcccctgtggccttggggccaattttcaccagaccgcgatagctattttgggagtggccggccgaaatgcccttagagacaatcctgctgcgggtatttttcccaatgtggatcatcttcgtgcccgtgtccgcctgttggtagtgattggtgagggcaacggagtaaaattctcccacggagttatcccccaccaagacacaactggggtatttccaagtaatggcagaacccgtctccacctgtgtccaggaaatcttggaattgcggccgaggcagagtccccgcttcgtcacaaagttgtaaatgccccctttgccattttcatcgccggcataccagttttgcacagtggagtatttgatttcggcattgtccagagccaccagctccaccactgccgcatggagttgattggtgtcaaacatgggagcagtacaaccctcaagatagctcacgtagctcccggcatcggcaatgatcagggtgcgctcaaactgacccgactcaccgttattgatgcggaaataggtggatagctccattggacagcgggtattcttgggaacatagacgaaggagccatcggaaaaaactgcggagttcaaggcagcatagaaattatcgccaatgggaacaacactgcctaagtatttctgcactaactcgggatagtcctggagcgcttcagaaatggagcaaaaaatgatcccctgcttggccaactcctcgcggaaggtggtggccactgacacactatcgaaaatggcatctacggctacattggtgagccgcttttgctctgaaaggggaatccctagtttttcaaaggtttccagcagaacgggatctacttcatccaagctttttagcttttccttctgtttcggagctgagtaatagacgatgtcttgataattgatggggggatagctcacccgtggccattggggctcgctcatcttcagccattgacgataggcacgcaggcgaaactccagcatgaactctggctcgttcttcttggcggagatgaggcgaataatgtcctcgttgagacctttgggaatggtttccgtctcaatgggggtg

SEQ ID NO: 3 - DNA, 50 bp, Artificial Sequence
  Description of Artificial Sequence Synthetic oligonucleotide
taacaccgtgcgtgttgactattttacctctggcggtgataatggttgca

SEQ ID NO: 4 - DNA, 762 bp, Artificial Sequence
  Description of Artificial Sequence Synthetic polynucleotide
atgaaaggaccaataataatgactagagaagaaagaatgaagattgttcatgaaattaaggaacgaatattggataaatatggggatgatgttaaggcaattggtgtttatggctctcttggtcgtcagactgatgggccctattcggatattgagatgatgtgtgttctgtcaacagagggagtagagttcagctatgaatggacaaccggtgagtggaaggcggaagtgaatttttatagcgaagagattctactagattatgcatctcgggtggaaccggattggccgcttacacatggtcgatttttctctattttgccgatttatgatccaggtggatactttgagaaagtgtaccaaactgctaaatcggtagaagcccaaaagttccacgatgcgatctgtgcccttatcgtagaagagctgtttgaatatgcaggcaaatggcgtaatattcgtgtgcaaggaccgacaacatttctaccatccttgactgtacaggtggcaatggcaggtgccatgttgattggtctgcatcatcgcatctgttatacgacgagcgcttcggtcttaactgaagcagttaagcaaccagatcttcctccaggttatgtccaactgtgccagctcgtaatgtctggtcaactttccgaccctgagaaacttctggaatcgctagagaatttctggaatggggttcaggagtgggcggaacgacacggatatatagtggatgtgtcaaaacgcataccattttga

SEQ ID NO: 5 - DNA, 4224 bp, Artificial Sequence
  Description of Artificial Sequence Synthetic polynucleotide
ctagaggagcttgttaacaggcttacggctgttggcggcagcaacgcgcttaccccatttgaccaattcttcagtgcagtcttcacgaccgatgaagcattcgatcagggttgggccgtcggtgtttgccagagcaaccttgatagcttctgccagttcgccaccggttttagccttcaggcctttaccagcaccgctgtcataaccaccgttaccgttgaacacttccatcagaccggcataatcccagttcttgatgttgttgtacggaccatcatggatcataacttcgatggtgtaaccatagttattgatcaagaagatgataaccggcagtttcaggcgaaccatctgagcgacttcctgagccgtcagctggaaggaaccatcaccaaccatgaggatgttgcgacgttccggagcaccgacggcataaccgaaggcggcaggaacggaccaaccgatgtgaccccactgcatttcatattcaacgcgagcaccgttcgggagcttcatgcgctgagcattgaaccaagagtcaccggtttcagcaataaccgtcgtgttcggggtcagaagagcttcgacctgacgggcgatttctgcgttgaccaacggagcactcggatcagccggagcggctttcttcagttcacctgcattgagggatttgaagaagtccaaagcaccggttttcttggaaactttctgagccaaacgggtcagatagtctttcagatgaacgctggggaagcgaacgccgttaacgacgacagaacgcggttcagcgagaaccagtttcttaggatcaggaatatccgtccaaccagtggtggagtagtcgttgaagacaggagccagagcgataaccgcatcggcttctttcatcgtcttttcaacgcccggatagctgacttcaccccatgaggtaccgatgtaatgcgggttttcttctgggaagaagctttttgcagcagccatggtagcaactgcgccaccgagagcatcagcaaatttgacagcagcttcttcagcaccagctgcgcgcagcttgctgccgacgaggacggcaactttgtcgcggttggcgatgaatttcagggtttcttcaaccgctgcattcaaagaagcttcgtcgctggcttcgtcattgaacaatgcgcttgccggtccaggagcggcgcagggcatggaagcaatgttgcaagcgatttcgagataaaccggcttcttctcacgaagagcagttttaatcacgtgatcgattttagccggagcttcttctggggtgtaaatcgcttcagctgcggccgtgatgttcttggccatttccaactgatagtgatagtcggttttgccaagagcgtgatgcaacacgtgaccagcagcgtgatcattgttgttcggagcaccggagatcaggataaccggaaggttttctgcataggcgccaccgatagcatcaaatgcggaaagcgcaccgacgctgtaggtaacgacggctgctgctgcgcctttggcacgagcataaccttctgcactgaaaccgcagttcagttcgttacagcaataaacctgctccatgtttttgttcaaaagcaggttgtcaagaaggacgaggttgtagtcgcccgcgactgcgaagtgatgcttgagaccaatctggacaagccgctccgctaaataggtaccgacagtataactcatatgttttcctccagcaaaaggatcctgcaaccattatcaccgccagaggtaaaatagtcaacacgcacggtgttaggccgcataggccagaggcgcgcctggccttcatggcctataaacgcagaaaggcccacccgaaggtgagccagtgtgactctagtagagagcgttcaccgacaaacaacagataaaacgaaaggcccagtctttcgactgagcctttcgttttatttgatgcctggaatacttcgaagagatgctcgacgtccgtatctcaggctagcttagaagaactcatccagcagacggtagaaggcaatgcgctgagaatccggcgctgcgataccgtacagcaccaggaaacggtcagcccattcaccacccagttcttctgcaatatcgcgggtagcgagggcgatatcctgatagcgatcagctacacccagacggccacagtcaataaaaccagagaagcggccgttttccaccataatgtttggcagacaagcgtcgccatgcgttaccaccaggtcttcgccgtccggcatgcgggctttcagacgtgcaaacagttccgccggtgcgaggccctggtgctcttcatccaggtcgtcctgatcaaccagacccgcttccatacgagtgcgtgcacgttcaatacggtgtttagcctgatggtcaaacgggcaagttgccgggtccagggtgtgcagacggcgcatcgcgtccgccatgatggaaactttttctgccggagcgaggtggctgctcagcagatcctgacccggaacttcacccagcagcagccaatcgcgaccggcttcagtaactacgtccagaactgccgcgcacggaacaccagtcgtcgcgagccaggacagacgggccgcttcgtcctgcagttcgttcagtgcgccggacaggtcggttttcacaaacagaaccggacgaccctgtgcagacagacggaaaaccgctgcatcgctacagccaatagtcagctgagcccagtcgtaaccaaacaggcgttccacccaagcagccggagaaccagcatgcaggccatcttgttcaatcatactcttcctttttcaatattattgaagcatttatcagggttattgtctcatgagcagatacatatttgaatgtatttagaaaaataaacaaataggggtcgggccggcgataatacgccggcccgttttttttggccatgaaggccaggcgcgcctctggcctatgcggcctgttgacaattaatcatcggcatagtatatcggcatagtataatacgacaaggtgaggaactaacatatgtgggaaactaagattaatatcaacgaagtccgtgagatccgcgcgaaaaccaccgtttactttggtgttggtgctatcaagaaaattgatgatatcgctcgcgagttcaaagaaaaaggttacgatcgcatcatcgtgatcaccggtaaaggcgcttacaaagcgaccggtgcatgggaatacatcgtgcctgctctgaacaaaaaccagattacgtatatccattatgatcaggtgaccccgaacccgaccgtagatcaggttgacgaagcgaccaaacaggcccgtgaatttggcgctcgcgcagtactggctattggtggcggttccccgatcgacgcagccaaatctgtggcggtgctgctgtcttatccggacaaaaacgctcgtcagctgtaccagctggagtttaccccggtaaaagcagcgccgatcatcgccatcaacctgacccacggtacgggcaccgaagcggaccgcttcgcggttgtatctatcccggagaaggcctacaaaccggctatcgcttacgattgcatctacccgctgtactctattgacgacccggctctgatggttaaactgccgagcgaccagacggcgtacgttagcgtggatgccctgaaccatgttgttgaagctgcgacctccaaagttgcatctccgtacactattatcctggcaaaagaaacggtccgtctcatcgcacgctacctgcctcaggccctgtctcaccctgcagacctgaccgcgcgttattacctcctgtatgcctctctgatcgccggtattgcgtttgataacggcctgctgcatttcacccacgcactggaacacccgctgtctgccgtgaaacctgaactggctcatggcctgggtctgggtatgctcctgcctgcggtagttaaacaaatttatccggctaccccggaggtactggcggaaatcctggaaccaatcgtaccggatctgaaaggcgttccgggcgaggctgagaaagcggcgtctggcgtggcgaaatggctggctggtgcaggcatcactatgaaactgaaagacgcgggtttccaggctgaagatatcgcgcgtctgaccgacctggccttcaccactccatccctggaactcctgctgtctatggcaccagtaactgctgatcgtgagcgtgtgaaagcaatttaccaggacgcattttga

SEQ ID NO: 6 - DNA, 4224 bp, Artificial Sequence
  Description of Artificial Sequence Synthetic polynucleotide
ctagaggagcttgttaacaggcttacggctgttggcggcagcaacgcgcttaccccatttgaccaattcttcagtgcagtcttcacgaccgatgaagcattcgatcagggttgggccgtcggtgtttgccagagcaaccttgatagcttctgccagttcgccaccggttttagccttcaggcctttaccagcaccgctgtcataaccaccgttaccgttgaacacttccatcagaccggcataatcccagttcttgatgttgttgtacggaccatcatggatcataacttcgatggtgtaaccatagttattgatcaagaagatgataaccggcagtttcaggcgaaccatctgagcgacttcctgagccgtcagctggaaggaaccatcaccaaccatgaggatgttgcgacgttccggagcaccgacggcataaccgaaggcggcaggaacggaccaaccgatgtgaccccactgcatttcatattcaacgcgagcaccgttcgggagcttcatgcgctgagcattgaaccaagagtcaccggtttcagcaataaccgtcgtgttcggggtcagaagagcttcgacctgacgggcgatttctgcgttgaccaacggagcactcggatcagccggagcggctttcttcagttcacctgcattgagggatttgaagaagtccaaagcaccggttttcttggaaactttctgagccaaacgggtcagatagtctttcagatgaacgctggggaagcgaacgccgttaacgacgacagaacgcggttcagcgagaaccagtttcttaggatcaggaatatccgtccaaccagtggtggagtagtcgttgaagacaggagccagagcgataaccgcatcggcttctttcatcgtcttttcaacgcccggatagctgacttcaccccatgaggtaccgatgtaatgcgggttttcttctgggaagaagctttttgcagcagccatggtagcaactgcgccaccgagagcatcagcaaatttgacagcagcttcttcagcaccagctgcgcgcagcttgctgccgacgaggacggcaactttgtcgcggttggcgatgaatttcagggtttcttcaaccgctgcattcaaagaagcttcgtcgctggcttcgtcattgaacaatgcgcttgccggtccaggagcggcgcagggcatggaagcaatgttgcaagcgatttcgagataaaccggcttcttctcacgaagagcagttttaatcacgtgatcgattttagccggagcttcttctggggtgtaaatcgcttcagctgcggccgtgatgttcttggccatttccaactgatagtgatagtcggttttgccaagagcgtgatgcaacacgtgaccagcagcgtgatcattgttgttcggagcaccggagatcaggataaccggaaggttttctgcataggcgccaccgatagcatcaaatgcggaaagcgcaccgacgctgtaggtaacgacggctgctgctgcgcctttggcacgagcataaccttctgcactgaaaccgcagttcagttcgttacagcaataaacctgctccatgtttttgttcaaaagcaggttgtcaagaaggacgaggttgtagtcgcccgcgactgcgaagtgatgcttgagaccaatctggacaagccgctccgctaaataggtaccgacagtataactcatatgttagttcctcaccttgtcgtattatactatgccgatatactatgccgatgattaattgtcaacaggccgcataggccagaggcgcgcctggccttcatggccaaaaaaaacgggccggcgtattatcgccggcccgacccctatttgtttatttttctaaatacattcaaatatgtatctgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaagctagcctgagatacggacgtcgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataggccatgaaggccaggcgcgcctctggcctatgcggcctaacaccgtgcgtgttgactattttacctctggcggtgataatggttgcaggatccttttgctggaggaaaacatatgtgggaaactaagattaatatcaacgaagtccgtgagatccgcgcgaaaaccaccgtttactttggtgttggtgctatcaagaaaattgatgatatcgctcgcgagttcaaagaaaaaggttacgatcgcatcatcgtgatcaccggtaaaggcgcttacaaagcgaccggtgcatgggaatacatcgtgcctgctctgaacaaaaaccagattacgtatatccattatgatcaggtgaccccgaacccgaccgtagatcaggttgacgaagcgaccaaacaggcccgtgaatttggcgctcgcgcagtactggctattggtggcggttccccgatcgacgcagccaaatctgtggcggtgctgctgtcttatccggacaaaaacgctcgtcagctgtaccagctggagtttaccccggtaaaagcagcgccgatcatcgccatcaacctgacccacggtacgggcaccgaagcggaccgcttcgcggttgtatctatcccggagaaggcctacaaaccggctatcgcttacgattgcatctacccgctgtactctattgacgacccggctctgatggttaaactgccgagcgaccagacggcgtacgttagcgtggatgccctgaaccatgttgttgaagctgcgacctccaaagttgcatctccgtacactattatcctggcaaaagaaacggtccgtctcatcgcacgctacctgcctcaggccctgtctcaccctgcagacctgaccgcgcgttattacctcctgtatgcctctctgatcgccggtattgcgtttgataacggcctgctgcatttcacccacgcactggaacacccgctgtctgccgtgaaacctgaactggctcatggcctgggtctgggtatgctcctgcctgcggtagttaaacaaatttatccggctaccccggaggtactggcggaaatcctggaaccaatcgtaccggatctgaaaggcgttccgggcgaggctgagaaagcggcgtctggcgtggcgaaatggctggctggtgcaggcatcactatgaaactgaaagacgcgggtttccaggctgaagatatcgcgcgtctgaccgacctggccttcaccactccatccctggaactcctgctgtctatggcaccagtaactgctgatcgtgagcgtgtgaaagcaatttaccaggacgcattttga

SEQ ID NO: 7 - DNA, 4289 bp, Artificial Sequence
  Description of Artificial Sequence Synthetic polynucleotide
ctagaggagcttgttaacaggcttacggctgttggcggcagcaacgcgcttaccccatttgaccaattcttcagtgcagtcttcacgaccgatgaagcattcgatcagggttgggccgtcggtgtttgccagagcaaccttgatagcttctgccagttcgccaccggttttagccttcaggcctttaccagcaccgctgtcataaccaccgttaccgttgaacacttccatcagaccggcataatcccagttcttgatgttgttgtacggaccatcatggatcataacttcgatggtgtaaccatagttattgatcaagaagatgataaccggcagtttcaggcgaaccatctgagcgacttcctgagccgtcagctggaaggaaccatcaccaaccatgaggatgttgcgacgttccggagcaccgacggcataaccgaaggcggcaggaacggaccaaccgatgtgaccccactgcatttcatattcaacgcgagcaccgttcgggagcttcatgcgctgagcattgaaccaagagtcaccggtttcagcaataaccgtcgtgttcggggtcagaagagcttcgacctgacgggcgatttctgcgttgaccaacggagcactcggatcagccggagcggctttcttcagttcacctgcattgagggatttgaagaagtccaaagcaccggttttcttggaaactttctgagccaaacgggtcagatagtctttcagatgaacgctggggaagcgaacgccgttaacgacgacagaacgcggttcagcgagaaccagtttcttaggatcaggaatatccgtccaaccagtggtggagtagtcgttgaagacaggagccagagcgataaccgcatcggcttctttcatcgtcttttcaacgcccggatagctgacttcaccccatgaggtaccgatgtaatgcgggttttcttctgggaagaagctttttgcagcagccatggtagcaactgcgccaccgagagcatcagcaaatttgacagcagcttcttcagcaccagctgcgcgcagcttgctgccgacgaggacggcaactttgtcgcggttggcgatgaatttcagggtttcttcaaccgctgcattcaaagaagcttcgtcgctggcttcgtcattgaacaatgcgcttgccggtccaggagcggcgcagggcatggaagcaatgttgcaagcgatttcgagataaaccggcttcttctcacgaagagcagttttaatcacgtgatcgattttagccggagcttcttctggggtgtaaatcgcttcagctgcggccgtgatgttcttggccatttccaactgatagtgatagtcggttttgccaagagcgtgatgcaacacgtgaccagcagcgtgatcattgttgttcggagcaccggagatcaggataaccggaaggttttctgcataggcgccaccgatagcatcaaatgcggaaagcgcaccgacgctgtaggtaacgacggctgctgctgcgcctttggcacgagcataaccttctgcactgaaaccgcagttcagttcgttacagcaataaacctgctccatgtttttgttcaaaagcaggttgtcaagaaggacgaggttgtagtcgcccgcgactgcgaagtgatgcttgagaccaatctggacaagccgctccgctaaataggtaccgacagtataactcatatgttttcctccagcaaaaggatcctgcaaccattatcaccgccagaggtaaaatagtcaacacgcacggtgttaggccgcataggccagaggcgcgcctggccttcatggcctataaacgcagaaaggcccacccgaaggtgagccagtgtgactctagtagagagcgttcaccgacaaacaacagataaaacgaaaggcccagtctttcgactgagcctttcgttttatttgatgcctggaatacttcgaagagatgctcgacgtccgtatctcaggctagcttagaagaactcatccagcagacggtagaaggcaatgcgctgagaatccggcgctgcgataccgtacagcaccaggaaacggtcagcccattcaccacccagttcttctgcaatatcgcgggtagcgagggcgatatcctgatagcgatcagctacacccagacggccacagtcaataaaaccagagaagcggccgttttccaccataatgtttggcagacaagcgtcgccatgcgttaccaccaggtcttcgccgtccggcatgcgggctttcagacgtgcaaacagttccgccggtgcgaggccctggtgctcttcatccaggtcgtcctgatcaaccagacccgcttccatacgagtgcgtgcacgttcaatacggtgtttagcctgatggtcaaacgggcaagttgccgggtccagggtgtgcagacggcgcatcgcgtccgccatgatggaaactttttctgccggagcgaggtggctgctcagcagatcctgacccggaacttcacccagcagcagccaatcgcgaccggcttcagtaactacgtccagaactgccgcgcacggaacaccagtcgtcgcgagccaggacagacgggccgcttcgtcctgcagttcgttcagtgcgccggacaggtcggttttcacaaacagaaccggacgaccctgtgcagacagacggaaaaccgctgcatcgctacagccaatagtcagctgagcccagtcgtaaccaaacaggcgttccacccaagcagccggagaaccagcatgcaggccatcttgttcaatcatactcttcctttttcaatattattgaagcatttatcagggttattgtctcatgagcagatacatatttgaatgtatttagaaaaataaacaaataggggtcgggccggcgataatacgccggcccgttttttttggccatgaaggccaggcgcgcctctggcctatgcggcctcgccctcattttctccctaggaggggcttcgatgcaaaaattgcccgaggtgttgacaaacgctcagggtattcgctacattaactaatgctgagtcttgatctaaagatctttctagattctcgaggcatatgtgggaaactaagattaatatcaacgaagtccgtgagatccgcgcgaaaaccaccgtttactttggtgttggtgctatcaagaaaattgatgatatcgctcgcgagttcaaagaaaaaggttacgatcgcatcatcgtgatcaccggtaaaggcgcttacaaagcgaccggtgcatgggaatacatcgtgcctgctctgaacaaaaaccagattacgtatatccattatgatcaggtgaccccgaacccgaccgtagatcaggttgacgaagcgaccaaacaggcccgtgaatttggcgctcgcgcagtactggctattggtggcggttccccgatcgacgcagccaaatctgtggcggtgctgctgtcttatccggacaaaaacgctcgtcagctgtaccagctggagtttaccccggtaaaagcagcgccgatcatcgccatcaacctgacccacggtacgggcaccgaagcggaccgcttcgcggttgtatctatcccggagaaggcctacaaaccggctatcgcttacgattgcatctacccgctgtactctattgacgacccggctctgatggttaaactgccgagcgaccagacggcgtacgttagcgtggatgccctgaaccatgttgttgaagctgcgacctccaaagttgcatctccgtacactattatcctggcaaaagaaacggtccgtctcatcgcacgctacctgcctcaggccctgtctcaccctgcagacctgaccgcgcgttattacctcctgtatgcctctctgatcgccggtattgcgtttgataacggcctgctgcatttcacccacgcactggaacacccgctgtctgccgtgaaacctgaactggctcatggcctgggtctgggtatgctcctgcctgcggtagttaaacaaatttatccggctaccccggaggtactggcggaaatcctggaaccaatcgtaccggatctgaaaggcgttccgggcgaggctgagaaagcggcgtctggcgtggcgaaatggctggctggtgcaggcatcactatgaaactgaaagacgcgggtttccaggctgaagatatcgcgcgtctgaccgacctggccttcaccactccatccctggaactcctgctgtctatggcaccagtaactgctgatcgtgagcgtgtgaaagcaatttaccaggacgcattttga

SEQ ID NO: 8 - DNA, 4289 bp, Artificial Sequence
  Description of Artificial Sequence Synthetic polynucleotide
ctagaggagcttgttaacaggcttacggctgttggcggcagcaacgcgcttaccccatttgaccaattcttcagtgcagtcttcacgaccgatgaagcattcgatcagggttgggccgtcggtgtttgccagagcaaccttgatagcttctgccagttcgccaccggttttagccttcaggcctttaccagcaccgctgtcataaccaccgttaccgttgaacacttccatcagaccggcataatcccagttcttgatgttgttgtacggaccatcatggatcataacttcgatggtgtaaccatagttattgatcaagaagatgataaccggcagtttcaggcgaaccatctgagcgacttcctgagccgtcagctggaaggaaccatcaccaaccatgaggatgttgcgacgttccggagcaccgacggcataaccgaaggcggcaggaacggaccaaccgatgtgaccccactgcatttcatattcaacgcgagcaccgttcgggagcttcatgcgctgagcattgaaccaagagtcaccggtttcagcaataaccgtcgtgttcggggtcagaagagcttcgacctgacgggcgatttctgcgttgaccaacggagcactcggatcagccggagcggctttcttcagttcacctgcattgagggatttgaagaagtccaaagcaccggttttcttggaaactttctgagccaaacgggtcagatagtctttcagatgaacgctggggaagcgaacgccgttaacgacgacagaacgcggttcagcgagaaccagtttcttaggatcaggaatatccgtccaaccagtggtggagtagtcgttgaagacaggagccagagcgataaccgcatcggcttctttcatcgtcttttcaacgcccggatagctgacttcaccccatgaggtaccgatgtaatgcgggttttcttctgggaagaagctttttgcagcagccatggtagcaactgcgccaccgagagcatcagcaaatttgacagcagcttcttcagcaccagctgcgcgcagcttgctgccgacgaggacggcaactttgtcgcggttggcgatgaatttcagggtttcttcaaccgctgcattcaaagaagcttcgtcgctggcttcgtcattgaacaatgcgcttgccggtccaggagcggcgcagggcatggaagcaatgttgcaagcgatttcgagataaaccggcttcttctcacgaagagcagttttaatcacgtgatcgattttagccggagcttcttctggggtgtaaatcgcttcagctgcggccgtgatgttcttggccatttccaactgatagtgatagtcggttttgccaagagcgtgatgcaacacgtgaccagcagcgtgatcattgttgttcggagcaccggagatcaggataaccggaaggttttctgcataggcgccaccgatagcatcaaatgcggaaagcgcaccgacgctgtaggtaacgacggctgctgctgcgcctttggcacgagcataaccttctgcactgaaaccgcagttcagttcgttacagcaataaacctgctccatgtttttgttcaaaagcaggttgtcaagaaggacgaggttgtagtcgcccgcgactgcgaagtgatgcttgagaccaatctggacaagccgctccgctaaataggtaccgacagtataactcatatgcctcgagaatctagaaagatctttagatcaagactcagcattagttaatgtagcgaataccctgagcgtttgtcaacacctcgggcaatttttgcatcgaagcccctcctagggagaaaatgagggcgaggccgcataggccagaggcgcgcctggccttcatggccaaaaaaaacgggccggcgtattatcgccggcccgacccctatttgtttatttttctaaatacattcaaatatgtatctgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaagctagcctgagatacggacgtcgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataggccatgaaggccaggcgcgcctctggcctatgcggcctaacaccgtgcgtgttgactattttacctctggcggtgataatggttgcaggatccttttgctggaggaaaacatatgtgggaaactaagattaatatcaacgaagtccgtgagatccgcgcgaaaaccaccgtttactttggtgttggtgctatcaagaaaattgatgatatcgctcgcgagttcaaagaaaaaggttacgatcgcatcatcgtgatcaccggtaaaggcgcttacaaagcgaccggtgcatgggaatacatcgtgcctgctctgaacaaaaaccagattacgtatatccattatgatcaggtgaccccgaacccgaccgtagatcaggttgacgaagcgaccaaacaggcccgtgaatttggcgctcgcgcagtactggctattggtggcggttccccgatcgacgcagccaaatctgtggcggtgctgctgtcttatccggacaaaaacgctcgtcagctgtaccagctggagtttaccccggtaaaagcagcgccgatcatcgccatcaacctgacccacggtacgggcaccgaagcggaccgcttcgcggttgtatctatcccggagaaggcctacaaaccggctatcgcttacgattgcatctacccgctgtactctattgacgacccggctctgatggttaaactgccgagcgaccagacggcgtacgttagcgtggatgccctgaaccatgttgttgaagctgcgacctccaaagttgcatctccgtacactattatcctggcaaaagaaacggtccgtctcatcgcacgctacctgcctcaggccctgtctcaccctgcagacctgaccgcgcgttattacctcctgtatgcctctctgatcgccggtattgcgtttgataacggcctgctgcatttcacccacgcactggaacacccgctgtctgccgtgaaacctgaactggctcatggcctgggtctgggtatgctcctgcctgcggtagttaaacaaatttatccggctaccccggaggtactggcggaaatcctggaaccaatcgtaccggatctgaaaggcgttccgggcgaggctgagaaagcggcgtctggcgtggcgaaatggctggctggtgcaggcatcactatgaaactgaaagacgcgggtttccaggctgaagatatcgcgcgtctgaccgacctggccttcaccactccatccctggaactcctgctgtctatggcaccagtaactgctgatcgtgagcgtgtgaaagcaatttaccaggacgcattttga

SEQ ID NO: 9 - DNA, 3096 bp, Artificial Sequence
  Description of Artificial Sequence Synthetic polynucleotide
gcggccgcgggggggggggggaaagccacgttgtgtctcaaaatctctgatgttacattgcacaagataaaaatatatcatcatgaacaataaaactgtctgcttacataaacagtaatacaaggggtcatatgtataccgttggtatgtacttggcagaacgcctagcccagatcggcctgaaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggtgatggctctttccagctgactgctcaagaagttgctcagatgatccgctatgaaatcccggtcatcatcttcctgatcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaattaactcgagtaacaccgtgcgtgttgactattttacctctggcggtgataatggttgcaggatccttttgctggaggaaaaccatatgtgggaaactaagattaatatcaacgaagtccgtgagatccgcgcgaaaaccaccgtttactttggtgttggtgctatcaagaaaattgatgatatcgctcgcgagttcaaagaaaaaggttacgatcgcatcatcgtgatcaccggtaaaggcgcttacaaagcgaccggtgcatgggaatacatcgtgcctgctctgaacaaaaaccagattacgtatatccattatgatcaggtgaccccgaacccgaccgtagatcaggttgacgaagcgaccaaacaggcccgtgaatttggcgctcgcgcagtactggctattggtggcggttccccgatcgacgcagccaaatctgtggcggtgctgctgtcttatccggacaaaaacgctcgtcagctgtaccagctggagtttaccccggtaaaagcagcgccgatcatcgccatcaacctgacccacggtacgggcaccgaagcggaccgcttcgcggttgtatctatcccggagaaggcctacaaaccggctatcgcttacgattgcatctacccgctgtactctattgacgacccggctctgatggttaaactgccgagcgaccagacggcgtacgttagcgtggatgccctgaaccatgttgttgaagctgcgacctccaaagttgcatctccgtacactattatcctggcaaaagaaacggtccgtctcatcgcacgctacctgcctcaggccctgtctcaccctgcagacctgaccgcgcgttattacctcctgtatgcctctctgatcgccggtattgcgtttgataacggcctgctgcatttcacccacgcactggaacacccgctgtctgccgtgaaacctgaactggctcatggcctgggtctgggtatgctcctgcctgcggtagttaaacaaatttatccggctaccccggaggtactggcggaaatcctggaaccaatcgtaccggatctgaaaggcgttccgggcgaggctgagaaagcggcgtctggcgtggcgaaatggctggctggtgcaggcatcactatgaaactgaaagacgcgggtttccaggctgaagatatcgcgcgtctgaccgacctggccttcaccactccatccctggaactcctgctgtctatggcaccagtaactgctgatcgtgagcgtgtgaaagcaatttaccaggacgcattttgagcggccgc

SEQ ID NO: 10 - DNA, 3567 bp, Artificial Sequence
  Description of Artificial Sequence Synthetic polynucleotide
gcggccgcttcgttataaaataaacttaacaaatctatacccacctgtagagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaatcatatgtataccgttggtatgtacttggcagaacgcctagcccagatcggcctgaaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggtgatggctctttccagctgactgctcaagaagttgctcagatgatccgctatgaaatcccggtcatcatcttcctgatcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaattaactcgagtaacaccgtgcgtgttgactattttacctctggcggtgataatggttgcaggatccttttgctggaggaaaaccatatgtgggaaactaagattaatatcaacgaagtccgtgagatccgcgcgaaaaccaccgtttactttggtgttggtgctatcaagaaaattgatgatatcgctcgcgagttcaaagaaaaaggttacgatcgcatcatcgtgatcaccggtaaaggcgcttacaaagcgaccggtgcatgggaatacatcgtgcctgctctgaacaaaaaccagattacgtatatccattatgatcaggtgaccccgaacccgaccgtagatcaggttgacgaagcgaccaaacaggcccgtgaatttggcgctcgcgcagtactggctattggtggcggttccccgatcgacgcagccaaatctgtggcggtgctgctgtcttatccggacaaaaacgctcgtcagctgtaccagctggagtttaccccggtaaaagcagcgccgatcatcgccatcaacctgacccacggtacgggcaccgaagcggaccgcttcgcggttgtatctatcccggagaaggcctacaaaccggctatcgcttacgattgcatctacccgctgtactctattgacgacccggctctgatggttaaactgccgagcgaccagacggcgtacgttagcgtggatgccctgaaccatgttgttgaagctgcgacctccaaagttgcatctccgtacactattatcctggcaaaagaaacggtccgtctcatcgcacgctacctgcctcaggccctgtctcaccctgcagacctgaccgcgcgttattacctcctgtatgcctctctgatcgccggtattgcgtttgataacggcctgctgcatttcacccacgcactggaacacccgctgtctgccgtgaaacctgaactggctcatggcctgggtctgggtatgctcctgcctgcggtagttaaacaaatttatccggctaccccggaggtactggcggaaatcctggaaccaatcgtaccggatctgaaaggcgttccgggcgaggctgagaaagcggcgtctggcgtggcgaaatggctggctggtgcaggcatcactatgaaactgaaagacgcgggtttccaggctgaagatatcgcgcgtctgaccgacctggccttcaccactccatccctggaactcctgctgtctatggcaccagtaactgctgatcgtgagcgtgtgaaagcaatttaccaggacgcattttgagcggccgc

SEQ ID NO: 11 - DNA, 3048 bp, Artificial Sequence
  Description of Artificial Sequence Synthetic polynucleotide
gcggccgcgggggggggggggaaagccacgttgtgtctcaaaatctctgatgttacattgcacaagataaaaatatatcatcatgaacaataaaactgtctgcttacataaacagtaatacaaggggtcatatgtataccgttggtatgtacttggcagaacgcctagcccagatcggcctgaaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggtgatggctctttccagctgactgctcaagaagttgctcagatgatccgctatgaaatcccggtcatcatcttcctgatcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaattaactcgagttggatcctataagtaggagataaacatatgtgggaaactaagattaatatcaacgaagtccgtgagatccgcgcgaaaaccaccgtttactttggtgttggtgctatcaagaaaattgatgatatcgctcgcgagttcaaagaaaaaggttacgatcgcatcatcgtgatcaccggtaaaggcgcttacaaagcgaccggtgcatgggaatacatcgtgcctgctctgaacaaaaaccagattacgtatatccattatgatcaggtgaccccgaacccgaccgtagatcaggttgacgaagcgaccaaacaggcccgtgaatttggcgctcgcgcagtactggctattggtggcggttccccgatcgacgcagccaaatctgtggcggtgctgctgtcttatccggacaaaaacgctcgtcagctgtaccagctggagtttaccccggtaaaagcagcgccgatcatcgccatcaacctgacccacggtacgggcaccgaagcggaccgcttcgcggttgtatctatcccggagaaggcctacaaaccggctatcgcttacgattgcatctacccgctgtactctattgacgacccggctctgatggttaaactgccgagcgaccagacggcgtacgttagcgtggatgccctgaaccatgttgttgaagctgcgacctccaaagttgcatctccgtacactattatcctggcaaaagaaacggtccgtctcatcgcacgctacctgcctcaggccctgtctcaccctgcagacctgaccgcgcgttattacctcctgtatgcctctctgatcgccggtattgcgtttgataacggcctgctgcatttcacccacgcactggaacacccgctgtctgccgtgaaacctgaactggctcatggcctgggtctgggtatgctcctgcctgcggtagttaaacaaatttatccggctaccccggaggtactggcggaaatcctggaaccaatcgtaccggatctgaaaggcgttccgggcgaggctgagaaagcggcgtctggcgtggcgaaatggctggctggtgcaggcatcactatgaaactgaaagacgcgggtttccaggctgaagatatcgcgcgtctgaccgacctggccttcaccactccatccctggaactcctgctgtctatggcaccagtaactgctgatcgtgagcgtgtgaaagcaatttaccaggacgcattttgagcggccgc

SEQ ID NO: 12 - DNA, 22 bp, Artificial Sequence
  Description of Artificial Sequence Synthetic primer
agatgccagattccgttaggtc

SEQ ID NO: 13 - DNA, 22 bp, Artificial Sequence
  Description of Artificial Sequence Synthetic primer
gattcatcgctttgcagatgtc

SEQ ID NO: 14 - DNA, 22 bp, Artificial Sequence
  Description of Artificial Sequence Synthetic primer
tctccagcaatttctcaagcag

SEQ ID NO: 15 - DNA, 22 bp, Artificial Sequence
  Description of Artificial Sequence Synthetic primer
tcagtctgacgaccaagagagc

SEQ ID NO: 16 - DNA, 22 bp, Artificial Sequence
  Description of Artificial Sequence Synthetic primer
aagcaaccagatcttcctccag

SEQ ID NO: 17 - DNA, 22 bp, Artificial Sequence
  Description of Artificial Sequence Synthetic primer
gggactgcccacctacagttac

SEQ ID NO: 18 - DNA, 22 bp, Artificial Sequence
  Description of Artificial Sequence Synthetic primer
ggatatttacgatgccctgacc

SEQ ID NO: 19 - DNA, 22 bp, Artificial Sequence
  Description of Artificial Sequence Synthetic primer
gtgttgagattctgcaccaagg

SEQ ID NO: 20 - DNA, 22 bp, Artificial Sequence
  Description of Artificial Sequence Synthetic primer
gagattcacgtcgaactcatgg

SEQ ID NO: 21 - DNA, 22 bp, Artificial Sequence
  Description of Artificial Sequence Synthetic primer
atccacctggatcataaatcgg

SEQ ID NO: 22 - DNA, 22 bp, Artificial Sequence
  Description of Artificial Sequence Synthetic primer
aagcaaccagatcttcctccag

SEQ ID NO: 23 - DNA, 22 bp, Artificial Sequence
  Description of Artificial Sequence Synthetic primer
gcaatacatcctgcatctgctc

(19) United States
(12) Patent Application Publication
(10) Pub. No.: US 2011/0020867 A1
(43) Pub. Date: Jan. 27, 2011

(54) Constructs And Methods For Efficient Transformation Of Micro-Organisms For Production Of Carbon-Based Products Of Interest

(75) Inventors: Nikos B. Reppas, Brookline, MA (US); Brian D. Green, Watertown, MA (US)

Correspondence Address: Joule/Fenwick, Silicon Valley Center, 801 California Street, Mountain View, CA, 94041, US

(73) Assignee: JOULE UNLIMITED, INC., Cambridge, MA (US)

(21) Appl. No.: 12/840,279

(22) Filed: Jul. 20, 2010

Related U.S. Application Data
(60) Provisional application No. 61/227,056, filed on Jul. 20, 2009.

(51) Int. Cl. C12P 7/06; C12P 7/62; C12P 19/12; C12P 7/02; C12P 7/04; C12P 7/16; C12P 7/64; C12P 5/00; C12P 7/44; C12P 17/08; C12P 17/10; C12P 7/48; C12P 17/02; C12P 21/00; C12N 1/21; C07H 21/04; C12N 15/74

(52) U.S. Cl. 435/69.1; 435/135; 435/100; 435/155; 435/161; 435/157; 435/160; 435/134; 435/166; 435/142; 435/124; 435/121; 435/144; 435/123; 435/252.3; 536/23.1; 435/471

(57) ABSTRACT

Improved constructs for increasing efficiency of transformation of thermophilic host cells for production of carbon-based products of interest and methods for producing carbon-based products of interest are provided.

### FIELD OF THE INVENTION

The present disclosure relates to mechanisms to confer production of carbon-based products to a photoautotrophic organism such that it efficiently converts carbon dioxide and light into various carbon-based products, and in particular the use of such organisms for the commercial production of various carbon-based products of interest.

### BACKGROUND

Photosynthesis is a process by which biological entities utilize sunlight and CO₂ to produce sugars for energy. Photosynthesis, as naturally evolved, is an extremely complex system with numerous and poorly understood feedback loops, control mechanisms, and process inefficiencies. This complicated system presents likely insurmountable obstacles to either one-factor-at-a-time or global optimization approaches [Nedbal L, Cerven Ã J, Rascher U, Schmidt H. E-photosynthesis: a comprehensive modeling approach to understand chlorophyll fluorescence transients and other complex dynamic features of photosynthesis in fluctuating light. Photosynth Res. 2007 July; 93(1-3):223-34; Salvucci M E, Crafts-Brandner S J. Inhibition of photosynthesis by heat stress: the activation state of Rubisco as a limiting factor in photosynthesis. Physiol Plant. 2004 February; 120(2):179-186; Greene D N, Whitney S M, Matsumura I. Artificially evolved Synechococcus PCC6301 Rubisco variants exhibit improvements in folding and catalytic efficiency. Biochem J. 2007 Jun. 15; 404(3):517-24].

Many existing photoautotrophic organisms (i.e., plants, algae, and photosynthetic bacteria) are poorly suited for industrial bioprocessing and have therefore not been used for this purpose. Said organisms have slow doubling time (3-72 hrs) compared to industrialized heterotrophic organisms such as Escherichia coli (20 minutes), reflective of low total productivities. In addition, techniques for genetic manipulation (knockout, over-expression of transgenes via integration or episomic plasmid propagation) of many of these organisms are inefficient, time-consuming, laborious, or non-existent. Thus a need exists for vectors and methods that can be used to genetically engineer organisms efficiently such that the organisms use photosynthesis to produce desired products, including biofuels and other carbon-based products.

### SUMMARY

The invention described herein provides constructs and methods to engineer thermophilic cyanobacteria to produce carbon-based products of interest.

In one embodiment, the method comprises preparing a heterologous DNA sequence operably linked to an expression vector; transforming a thermophilic cyanobacterium host with said vector; and culturing the host. Optionally, the method further comprises isolating the carbon-based product of interest from the host cell or a medium.

Also provided is a method for producing a biodiesel fuel composition, comprising preparing a heterologous DNA sequence operably linked to an expression vector; transforming a thermophilic cyanobacterium host with said vector; and culturing said host. Optionally, the method further comprises isolating the biodiesel fuel composition from the host cell or a medium.

In one embodiment, the carbon-based product of interest is selected from the group consisting of: ethyl ester, methyl ester, sucrose, alcohol, ethanol, propanol, isopropanol, butanol, fatty alcohols, fatty acid ester, wax ester, hydrocarbons, n-alkanes, propane, octane, diesel, JP8, polymers, terephthalate, polyol, 1,3-propanediol, 1,4-butanediol, PHA, PHB, acrylate, adipic acid, ε-caprolactone, isoprene, caprolactam, rubber, lactate, DHA, 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid, carotenoid, isoprenoid, itaconic acid, limonene, pharmaceutical or pharmaceutical intermediates, erythromycin 7-ADCA/cephalosporin, polyketides, statin, paclitaxel, docetaxel, terpene, peptide, steroid, and an omega fatty acid.

In certain embodiments, the host cell provided by the invention is capable of producing ethanol. In one embodiment, the carbon-based product of interest is ethanol, and the cyanobacterium produces at least 1000, at least 5000, at least 10,000, at least 12,000, or at least 15,000 mgs ethanol per liter of culture medium. In one embodiment, the carbon-based product of interest is ethanol, and the cyanobacterium produces between 1000 and 20,000 mgs ethanol per liter of culture medium. In one embodiment, the carbon-based product of interest is ethanol, and the cyanobacterium produces between 10,000 and 20,000, between 12,000 and 18,000, or between 13,000 and 16,000 mgs ethanol per liter of culture medium. In one embodiment, the carbon-based product of interest is ethanol, and the cyanobacterium further produces acetaldehyde, and wherein the ratio of ethanol to acetaldehyde is at least 500, at least 2000, at least 4000, at least 4500, at least 5000, at least 10,000, or between 4000 and 15,000, or between 500 and 3,000.

In yet other embodiments, thermophilic cyanobacteria engineered is Thermosynechococcus elongatus BP-1.

In another embodiment, transforming said thermophilic cyanobacterium host comprises with said vector comprises integrating at least a portion of said vector in a chromosome of said thermophilic cyanobacterium.

In other embodiments, a modified Thermosynechococcus cell comprising a recombinant marker gene and a λ phage cI promoter where in said marker gene is operably linked to said promoter is provided. In one embodiment the marker gene confers antibiotic resistance to said cell. In another embodiment the marker gene confers resistance to kanamycin to said cell. In yet another embodiment the marker gene is htk.

In yet another aspect, the invention provides an isolated or recombinant polynucleotide comprising or consisting of a nucleic acid sequence selected from the group consisting of: any one of the sequences from Table 3; a nucleic acid sequence at least 80%, at least 85%, at least 90%, at least 95%, at least 98%, at least 99% or at least 99.9% identical to any one of the sequences from Table 3; and a nucleic acid sequence that hybridizes under stringent conditions to any one of the sequences in Table 3.

In another embodiment, a modified Thermosynechococcus cell comprising an alcohol dehydrogenase gene and a pyruvate decarboxylase gene is provided. In one embodiment at least one of the genes is recombinant. In one embodiment the genes are divergently oriented. In one embodiment, the cell comprises at least one promoter. In one embodiment the at least on promoter is selected from the group consisting of tef, tac, trp, tet, trp-tet, lpp, lac, lpp-lac, lacIq, T7, T5, T3, gal, trc, ara, SP6, amyE, phage SP02, Pcpcb, PaphII, PtRNA_{Glu}, λ phage cI λ-p_{R} and λ-p_{L}. In one embodiment, the at least one promoter is PaphII.

In one embodiment the cell further comprises a first promoter operably linked to said alcohol dehydrogenase gene and a second promoter operably linked to said pyruvate decarboxylase gene. In one embodiment, the first promoter and said second promoter are each independently selected from the group consisting of tef, tac, trp, tet, trp-tet, lpp, lac, lpp-lac, lacIq, T7, T5, T3, gal, trc, ara, SP6, amyE, phage SP02, Pcpcb, PaphII, PtRNAGlu, λ phage cI λ-pR and λ-pL. In one embodiment at least one of said first promoter and said second promoter is λ phage cI. In one embodiment, the first promoter is λ phage cI and said second promoter is PEM7. In one embodiment, the first promoter is PEM7 and said second promoter is λ phage cI. In one embodiment, the first promoter is λ phage cI and said second promoter is PtRNAGlu. In one embodiment, the first promoter is PtRNAGlu and said second promoter is λ phage cI. In one embodiment, the first promoter is PaphII and said second promoter is λ phage cI. In one embodiment, the first promoter is Pcpcb and said second promoter is λ phage cI.

In one embodiment, the cell comprises any one of SEQ ID NO:5, SEQ ID NO:6, SEQ ID NO:7, SEQ ID NO:8, SEQ ID NO:9, SEQ ID NO:10 or SEQ ID NO: 11.

Also provided is a method producing a carbon-based product of interest by culturing the cell. In one embodiment, the carbon-based product of interest is selected from the group consisting of: ethyl ester, methyl ester, sucrose, alcohol, ethanol, propanol, isopropanol, butanol, fatty alcohols, fatty acid ester, wax ester, hydrocarbons, n-alkanes, propane, octane, diesel, JP8, polymers, terephthalate, polyol, 1,3-propanediol, 1,4-butanediol, PHA, PHB, acrylate, adipic acid, ε-caprolactone, isoprene, caprolactam, rubber, lactate, DHA, 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid, carotenoid, isoprenoid, itaconic acid, limonene, pharmaceutical or pharmaceutical intermediates, erythromycin 7-ADCA/cephalosporin, polyketides, statin, paclitaxel, docetaxel, terpene, peptide, steroid, and an omega fatty acid. In one embodiment, the carbon-based product of interest is an alcohol. In one embodiment, the carbon-based product of interest is ethanol.

In one embodiment, the carbon-based product of interest is ethanol, and the cyanobacterium produces at least 1000, at least 5000, at least 10,000, at least 12,000, or at least 15,000 mgs ethanol per liter of culture medium. In one embodiment, the carbon-based product of interest is ethanol, and the cyanobacterium produces between 1000 and 20,000 mgs ethanol per liter of culture medium. In one embodiment, the carbon-based product of interest is ethanol, and the cyanobacterium produces between 10,000 and 20,000, between 12,000 and 18,000, or between 13,000 and 16,000 mgs ethanol per liter of culture medium. In one embodiment, the carbon-based product of interest is ethanol, and the cyanobacterium further produces acetaldehyde, and wherein the ratio of ethanol to acetaldehyde is at least 500, at least 2000, at least 4000, at least 4500, at least 5000, at least 10,000, or between 4000 and 15,000, or between 500 and 3,000.

Also provided is a method of for engineering a thermophilic cyanobacterium comprising transforming said thermophilic cyanobacterium with a heterologous DNA sequence operably linked to an expression vector. expression vector comprises an isolated or recombinant polynucleotide comprising or consisting of a nucleic acid sequence selected from the group consisting of: any one of the sequences from Table 3; a nucleic acid sequence at least 80%, at least 85%, at least 90%, at least 95%, at least 98%, at least 99% or at least 99.9% identical to any one of the sequences from Table 3; and a nucleic acid sequence that hybridizes under stringent conditions to any one of the sequences in Table 3. In one embodiment the thermophilic cyanobacterium is Thermosynechococcus elongatus BP-1. In one embodiment, transforming the thermophilic cyanobacterium host comprises integrating at least a portion of said vector in a chromosome of said thermophilic cyanobacterium.

### BRIEF DESCRIPTION OF THE DRAWINGS

FIG. 1 provides gels illustrating successful transformation of host cells.

FIG. 2 is a diagram of pJB825 ethanologen constructs.

FIG. 3 is a diagram of pJB826 ethanologen constructs.

Table 1 provides primers useful for screening putative transformants to identify those actually transformed.

Table 2 provides data for acetaldehyde and ethanol production by transformed cells.

Table 3 provides an informal sequence listing.

Table 4 provides additional informal sequence listings.

### DETAILED DESCRIPTION

### Abbreviations and Terms

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a cell” includes one or a plurality of such cells, and so forth. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Accession Numbers: The accession numbers throughout this description are derived from the NCBI database (National Center for Biotechnology Information) maintained by the National Institute of Health, U.S.A. The accession numbers are as provided in the database on Jul. 15, 2009.

Enzyme Classification Numbers (EC): The EC numbers provided throughout this description are derived from the KEGG Ligand database, maintained by the Kyoto Encyclopedia of Genes and Genomics, sponsored in part by the University of Tokyo. The EC numbers are as provided in the database on Jul. 15, 2009.

Alcohol dehydrogenase is an enzyme that catalyzes the formation of an ethanol molecule by the reduction of acetaldehyde with nicotinamide adenine dinucleotide (NADH). The enzyme described herein is the class I alcohol dehydrogenase with zinc co-factor and is designated “ADH1.” The genes encoding the nucleotide sequences for the invention described herein is designated “adh1.”

Codons are triplets of nucleotides in DNA molecules and code for an amino acid. The term codon is also used for the corresponding (and complementary) sequences of three nucleotides in the mRNA into which the DNA sequence is transcribed.

Attenuate: The term as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non functional. In some instances a functional deletion is described as a knockout mutation. Attenuation also includes amino acid sequence changes by altering the nucleic acid sequence, placing the gene under the control of a less active promoter, downregulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non pathway specific feedback) is lessened such that the enzyme activity is not impacted by the presence of a compound. In other instances, an enzyme that has been altered to be less active can be referred to as attenuated.

Autotroph: Autotrophs (or autotrophic organisms) are organisms that produce complex organic compounds from simple inorganic molecules and an external source of energy, such as light (photoautotroph) or chemical reactions of inorganic compounds.

Biofuel: A biofuel is any fuel that derives from a biological source. Biofuel refers to one or more hydrocarbons, one or more alcohols, one or more fatty esters or a mixture thereof.

Biosynthetic pathway: Also referred to as “metabolic pathway,” refers to a set of anabolic or catabolic biochemical reactions for converting (transmuting) one chemical species into another. For example, a hydrocarbon biosynthetic pathway refers to the set of biochemical reactions that convert inputs and/or metabolites to hydrocarbon product like intermediates and then to hydrocarbons or hydrocarbon products. Anabolic pathways involve constructing a larger molecule from smaller molecules, a process requiring energy. Catabolic pathways involve breaking down of larger: molecules, often releasing energy.

“Carbon-based Products of Interest” include alcohols such as ethanol, propanol, isopropanol, butanol, fatty alcohols, fatty acid esters, wax esters; hydrocarbons and alkanes such as propane, octane, diesel, Jet Propellant 8 (JP8); polymers such as terephthalate, 1,3 propanediol, 1,4 butanediol, polyols, Polyhydroxyalkanoates (PHA), poly-beta-hydroxybutyrate (PHB), acrylate, adipic acid, ε caprolactone, isoprene, caprolactam, rubber; commodity chemicals such as lactate, Docosahexaenoic acid (DHA), 3 hydroxypropionate, γ valerolactone, lysine, serine, aspartate, aspartic acid, sorbitol, ascorbate, ascorbic acid, isopentenol, lanosterol, omega 3 DHA, lycopene, itaconate, 1,3 butadiene, ethylene, propylene, succinate, citrate, citric acid, glutamate, malate, 3-hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7 ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

Deletion: The removal of one or more nucleotides from a nucleic acid molecule or one or more amino acids from a protein, the regions on either side being joined together.

DNA: Deoxyribonucleic acid. DNA is a long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached.

Downregulation: When a gene is caused to be transcribed at a reduced rate compared to the endogenous gene transcription rate for that gene. In some examples, downregulation additionally includes a reduced level of translation of the gene compared to the endogenous translation rate for that gene. Methods of testing for downregulation are well known to those in the art, for example the transcribed RNA levels can be assessed using RT PCR and proteins levels can be assessed using SDS PAGE analysis.

Endogenous: As used herein with reference to a nucleic acid molecule and a particular cell or microorganism endogenous refers to a nucleic acid sequence or peptide that is in the cell and was not introduced into the cell (or its progentors) using recombinant engineering techniques. An example, a gene that was present in the cell when the cell was originally isolated from nature is endogenous. A gene is still considered endogenous if the control sequences, such as a promoter or enhancer sequences that activate transcription or translation have been altered through recombinant techniques.

The term “ethanologenesis” and “ethanologenic” as used herein with reference to a gene, gene product or protein capable of conferring on a host cell the capacity to produce, metabolically use or tolerate ethanol or is capable of improving any aspect of cellular production of ethanol, such as, e.g., substrate uptake, substrate processing, ethanol tolerance, etc. For instance, such genes include a gene encoding pyruvate decarboxylase and alcohol dehydrogenases I, II, III, IV, V and/or A, B, C.

Exogenous: As used herein with reference to a nucleic acid molecule and a particular cell or microorganism exogenous refers to a nucleic acid sequence or peptide that was not present in the cell when the cell was originally isolated from nature. For example, a nucleic acid that originated in a different microorganism and was engineered into an alternate cell using recombinant DNA techniques or other methods for delivering said nucleic acid is exogenous.

Expression: The process by which a gene's coded information is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Expression Control Sequence: as used herein refers to polynucleotide sequences which are necessary to affect the expression of coding sequences to which they are operatively linked. Expression control sequences are sequences which control the transcription, post transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Knock out: A gene whose level of expression or activity has been reduced to zero. In some examples, a gene is knocked out via deletion of some or all of its coding sequence. In other examples, a gene is knocked out via introduction of one or more nucleotides into its open reading frame, which results in translation of a non sense or otherwise non functional protein product.

Overexpression: When a gene is caused to be transcribed at an elevated rate compared to the endogenous transcription rate for that gene. In some examples, overexpression additionally includes an elevated rate of translation of the gene compared to the endogenous translation rate for that gene. Methods of testing for overexpression are well known in the art, for example transcribed RNA levels can be assessed using reverse transcriptase polymerase chain reaction (RT PCR) and protein levels can be assessed using sodium dodecyl sulfate polyacrylamide gel electrophoresis (SDS PAGE) analysis. Furthermore, a gene is considered to be overexpressed when it exhibits elevated activity compared to its endogenous activity, which may occur, for example, through reduction in concentration or activity of its inhibitor, or via expression of mutant version with elevated activity. In preferred embodiments, when the host cell encodes an endogenous gene with a desired biochemical activity, it is useful to overexpress an exogenous gene, which allows for more explicit regulatory control in the fermentation and a means to potentially mitigate the effects of central metabolism regulation, which is focused around the native genes explicitly.

“Fuel component” is any compound or a mixture of compounds that are used to formulate a fuel composition. There are “major fuel components” and “minor fuel components.” A major fuel component is present in a fuel composition by at least 50% by volume; and a minor fuel component is present in a fuel composition by less than 50%. Fuel additives are minor fuel components. The isoprenoid compounds disclosed herein can be a major component or a minor component, by themselves or in a mixture with other fuel components.

As used herein, a composition that is a “substantially pure” compound is substantially free of one or more other compounds, i.e., the composition contains greater than 80 vol. %, greater than 90 vol. %, greater than 95 vol. %, greater than 96 vol. %, greater than 97 vol. %, greater than 98 vol. %, greater than 99 vol. %, greater than 99.5 vol. %, greater than 99.6 vol. %, greater than 99.7 vol. %, greater than 99.8 vol. %, or greater than 99.9 vol. % of the compound; or less than 20 vol. %, less than 10 vol. %, less than 5 vol. %, less than 3 vol. %, less than 1 vol. %, less than 0.5 vol. %, less than 0.1 vol. %, or less than 0.01 vol. % of the one or more other compounds, based on the total volume of the composition.

Nucleic Acid Molecule: The term “nucleic acid molecule” of “polynucleotide” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation. If single stranded, the nucleic acid molecule can be the sense strand or the antisense strand.

Engineered nucleic acid: An “engineered nucleic acid” is a nucleic acid molecule that includes at least one difference from a naturally occurring nucleic acid molecule. An engineered nucleic acid includes all exogenous modified and unmodified heterologous sequences (i.e., sequences derived from an organism or cell other than that harboring the engineered nucleic acid) as well as endogenous genes, operons, coding sequences, or non coding sequences, that have been modified, mutated, or that include deletions or insertions as compared to a naturally occuring sequence. Engineered nucleic acids also include all sequences, regardless of origin, that are linked to an inducible promoter or to another control sequence with which they are not naturally associated.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

A particular, non-limiting example of a mathematical algorithm utilized for the comparison of sequences is that of Karlin and Altschul (Proc. Natl. Acad. Sci. (1990) USA 87:2264-68; Proc. Natl. Acad. Sci. USA (1993) 90: 5873-77) as used in the NBLAST and XBLAST programs (version 2.0) of Altschul et al. (J. Mol. Biol. (1990) 215:403-10). BLAST nucleotide searches can be performed with the NBLAST program, score=100, wordlength=12 to obtain nucleotide sequences homologous to nucleic acid molecules of the invention. BLAST polypeptide searches can be performed with the XBLAST program, score=50, wordlength=3 to obtain amino acid sequences homologous to polypeptide molecules of the invention. To obtain gapped alignments for comparison purposes, Gapped BLAST can be utilized as described in Altschul et al. (Nucleic Acids Research (1997) 25(17):3389-3402). When utilizing BLAST and Gapped BLAST programs, the default parameters of the respective programs (e.g., XBLAST and NBLAST) can be used (http://www.ncbi.nlm.nih.gov). One skilled in the art may also use the ALIGN program incorporating the non-linear algorithm of Myers and Miller (Comput. Appl. Biosci. (1988) 4:11-17). For amino acid sequence comparison using the ALIGN program one skilled in the art may use a PAM120 weight residue table, a gap length penalty of 12, and a gap penalty of 4.

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 71%, 72%, 73%, 74%, 75%, 76%, 77%, 78%, 79%, 80%, 81%, 82%, 83%, 84%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Intermediate ranges e.g., at 65-70° C. or at 42-50° C. are also within the scope of the invention. SSPE (1× SSPE is 0.15 M NaCl, 10 mM NaH₂PO₄, and 1.25 mM EDTA, pH 7.4) can be substituted for SSC (1×SSC is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41 (% G+C)−(600/N), where N is the number of bases in the hybrid, and [Na⁺] is the concentration of sodium ions in the hybridization buffer ([Na⁺] for 1×SSC=0.165 M).

The skilled practitioner recognizes that reagents can be added to hybridization and/or wash buffers. For example, to decrease non-specific hybridization of nucleic acid molecules to, for example, nitrocellulose or nylon membranes, blocking agents, including but not limited to, BSA or salmon or herring sperm carrier DNA and/or detergents, including but not limited to, SDS, chelating agents EDTA, Ficoll, PVP and the like can be used. When using nylon membranes, in particular, an additional, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (Church and Gilbert (1984) Proc. Natl. Acad. Sci. USA 81:1991-1995,) or, alternatively, 0.2×SSC, 1% SDS.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

Isolated: An “isolated” nucleic acid or polynucleotide (e.g., an RNA, DNA or a mixed polymer) is one which is substantially separated from other cellular components that naturally accompany the native polynucleotide in its natural host cell, e.g., ribosomes, polymerases, and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems. However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non native promoter sequence can be substituted (e.g. by homologous recombination) for the native promoter of a gene in the genome of a human cell, such that this gene has an altered expression pattern. This gene would now become “isolated” because it is separated from at least some of the sequences that naturally flank it. A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g. by human intervention. An “isolated nucleic acid” also includes a nucleic acid integrated into a host cell chromosome at a heterologous site, as well as a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame. Configurations of separate genes that are transcribed in tandem as a single messenger RNA are denoted as operons. Thus placing genes in close proximity, for example in a plasmid vector, under the transcriptional regulation of a single promoter, constitutes a synthetic operon.

Purified: The term purified does not require absolute purity; rather, it is intended as a relative term. Thus, for example, a purified product preparation, is one in which the product is more concentrated than the product is in its environment within a cell. For example, a purified wax is one that is substantially separated from cellular components (nucleic acids, lipids, carbohydrates, and other peptides) that can accompany it. In another example, a purified wax preparation is one in which the wax is substantially free from contaminants, such as those that might be present following fermentation.

Detectable: Capable of having an existence or presence ascertained using various analytical methods as described throughout the description or otherwise known to a person skilled in the art.

Microorganism: Includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

Recombinant: A recombinant nucleic acid molecule or protein is one that has a sequence that is not naturally occurring, has a sequence that is made by an artificial combination of two otherwise separated segments of sequence, or both. This artificial combination can be achieved, for example, by chemical synthesis or by the artificial manipulation of isolated segments of nucleic acid molecules or proteins, such as genetic engineering techniques. Recombinant is also used to describe nucleic acid molecules that have been artificially manipulated, but contain the same regulatory sequences and coding regions that are found in the organism from which the nucleic acid was isolated.

The term “recombinant host cell” (“expression host cell,” “expression host system,” “expression system,” or simply “host cell”), as used herein, refers to a cell into which a recombinant vector has been introduced, e.g., a vector comprising acyl CoA synthase. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

Release: The movement of a compound from inside a cell (intracellular) to outside a cell (extracellular). The movement can be active or passive. When release is active it can be facilitated by one or more transporter peptides and in some examples it can consume energy. When release is passive, it can be through diffusion through the membrane and can be facilitated by continually collecting the desired compound from the extracellular environment, thus promoting further diffusion. Release of a compound can also be accomplished by lysing a cell.

The terms “thermal stability” and “thermostability” are used interchangeably and refer to the ability of an enzyme (e.g., whether expressed in a cell, present in an cellular extract, cell lysate, or in purified or partially purified form) to exhibit the ability to catalyze a reaction at least at about 20° C., preferably at about 25° C. to 35° C., more preferably at about 37° C. or higher, in more preferably at about 50° C. or higher, and even more preferably at least about 60° C. or higher.

Vector: The term “vector” as used herein refers to a nucleic acid molecule capable of transporting another nucleic acid to which it has been linked. One type of vector is a “plasmid,” which refers to a circular double stranded DNA loop into which additional DNA segments may be ligated. Other vectors include cosmids, bacterial artificial chromosomes (BACs) and yeast artificial chromosomes (YACs). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”). A vector can also include one or more selectable marker genes and other genetic elements known in the art. Suitable vectors for use in cyanobacteria include self-replicating plasmids (e.g., multiple copy and high-level expression) and chromosomal integration plasmids. Integration of vectors into the host genome or autonomously replicating vectors allow for gene expression in the host cell. When stable expression results from integration, the site of the construct's integration can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

### General Methods for Engineering Microorganisms to Produce Carbon-Based Products

Generally, carbon-based products of interest are produced by expressing a gene or a set of genes in a photoautotrophic microorganism, e.g., cyanobacteria or thermophilic cyanobacteria as described herein. Plasmids are constructed to express various proteins that are useful in production of carbon-based products as described in Example 1. The constructs can be synthetically made or made using standard molecular biology methods and all the cloned genes are put under the control of constitutive promoters or inducible promoters. Plasmids containing the genes of interest are transformed into the host and corresponding transformants are selected in LB plate supplemented with antibiotics such as spectinomycin, carbenicillin, kanamycin, etc. Using standard molecular biology techniques, cells in which a nucleic acid molecule has been introduced are transformed to express or over-express desired genes while other nucleic acid molecules are attenuated or functionally deleted. Transformation techniques by which a nucleic acid molecule can be introduced into such a cell, including, but not limited to, transfection with viral vectors, conjugation, transformation with plasmid vectors, and introduction of naked DNA by electroporation, lipofection, and particle gun acceleration. Transformants are inoculated into a suitable medium. The samples containing the transformants are grown at suitable temperatures in a shaker until they reach at certain OD. The cells are then spun down at and the cell pellets are suspended. Separation techniques allows for the sample to be subjected to GC/MS analysis. Total yield is determined

### Selected or Engineered Microorganisms for the Production of Carbon-Based Products of Interest

A variety of host organisms can be transformed to produce a product of interest. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Cyanobacteria are photosynthetic bacteria which require light, inorganic elements, nitrogen sources, water and a carbon source, generally CO₂, to metabolize and grow. Cyanobacteria are photosynthetic prokaryotes which carry out oxygenic photosynthesis. The main product of the metabolic pathway of Cyanobacteria during aerobic conditions is oxygen and carbohydrates. Exemplary suitable cyanobacteria include those described in Donald Bryant, The Molecular Biology of Cyanobacteria, published by Kluwer Academic Publishers (1994).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera: Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis.

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospira sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic Sulfur-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

HyperPhotosynthetic conversion can require extensive genetic modification; in preferred embodiments the parental photoautotrophic organism can be transformed with exogenous DNA.

Preferred organisms for HyperPhotosynthetic conversion include: Arabidopsis thaliana, Panicum virgatum, Miscanthus giganteus, and Zea mays (plants), Botryococcus braunii, Chlamydomonas reinhardtii and Dunaliela salina (algae), Synechococcus sp PCC 7002, Synechococcus sp. PCC 7942, Synechocystis sp. PCC 6803, and Thermosynechococcus elongatus BP-1 (cyanobacteria), Chlorobium tepidum (green sulfur bacteria), Chloroflexus auranticus (green non-sulfur bacteria), Chromatium tepidum and Chromatium vinosum (purple sulfur bacteria), Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A common theme in selecting or engineering a suitable organism is autotrophic fixation of CO₂ to products. This would cover photosynthesis and methanogenesis. Acetogenesis, encompassing the three types of CO₂ fixation; Calvin cycle, acetyl CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways. Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in almost all aerobic autotrophic bacteria, for example, the cyanobacteria.

Additional inorganic carbon sources such as bicarbonate are also contemplated.

### Propagation of Selected Microoganisms

Methods for cultivation of photosynthetic organisms in liquid media and on agarose-containing plates are well known to those skilled in the art (see, e.g., websites associated with ATCC, and with the Institute Pasteur). For example, Thermosynechococcus elongatus BP-1 (available from the Kazusa DNAResearch Institute, Japan) is propagated in BG11 medium supplemented with 20 mM TES-KOH (pH 8.2) as described [Iwai M, Katoh H, Katayama M, Ikeuchi M. “Improved genetic transformation of the thermophilic cyanobacterium, Thermosynechococcus elongatus BP-1.” Plant Cell Physiol (2004). 45(2):171-175)]. Typically, cultures are maintained at 50° C. and bubbled continuously with 5% CO2 under a light intensity of 38 μmol photons/m2/s. T. elongatus BP-1 can also be grown in A⁺ medium. To date, however, thermophiles have not been suitable host cells for recombinant expression because of the difficulties associated in their transformation.

### Production of Carbon-Based Products of Interest

Herein is disclosed a method for transforming a thermophilic cyanobacterium. It is desirable for the host cell to achieve increased transformation efficiency and, thus, is optimized for use in a genetic system for production of various carbon-based products of interest.

In one embodiment, such a carbon-based product of interest is ethanol. In a preferred embodiment, the host cell produces commercial yields of ethanol. Ethanol has various commercial applications including use as a solvent, antiseptic, rocket propellant, renewable fuel source and as a base compound for the manufacture of other industrially important organic compounds. Therefore, it is desirable to increase the efficiency of the process whereby an organism is optimized for use in a genetic system for clean and efficient ethanol production.

Natural metabolic pathways for producing ethanol through fermentative processes are commonly found in plants, yeast and various fungi, while being less common in bacteria and entirely absent in animals. The enzyme activities required for the pyruvate decarboxylase pathway for producing ethanol are: pyruvate decarboxylase (EC 4.1.1.1) and alcohol dehydrogenase (EC 1.1.1.1 or EC 1.1.1.2). Pyruvate decarboxylase (PDC), only rarely found in bacteria, converts pyruvate to acetaldehyde by chemical reduction with NADH, with acetaldehyde also having important industrial applications. Alcohol dehydrogenase (ADH), more commonly found in a diverse array of bacterial organisms, converts acetaldehyde to ethanol. It has been demonstrated that an ethanol production metabolic pathway utilizing PDC and ADH can be engineered into microorganisms for the production of ethanol from nutrient rich growth media (Bräu and Sahm (1986) Arch. Microbiol. Vol. 144:296-301; U.S. Pat. No. 5,000,000; U.S. Pat. No. 5,028,539). Ethanol can then be isolated and used for other industrial applications as well as an alternative fuel source.

Accordingly, the invention includes improved constructs which may be utilized to more efficiently insert into a host cell genes such as those for expression of ADH and PDC.

In one embodiment, the invention includes producing ethanol using genetically engineered host cells into which genes for expression of ADH and PDC have been inserted by the improved constructs of the invention.

In alternative embodiments, methods for producing biodiesel are disclosed comprising: preparing a heterologous DNA sequence operably linked to an expression vector; transforming a thermophilic cyanobacterium host with said vector; and culturing said host. The thermophilic host may comprise various known pathways or be engineered to express synthetic pathways.

### Isolated or Recombinant Nucleic Acid Molecules

In various embodiments, the thermophilic host is suitable for recombinant expression of polynucleotides. Improved constructs and methods for increasing transformation efficiency of thermophilic host cells for the production of carbon-based products of interest are disclosed.

Accordingly, the present invention provides isolated or recombinant nucleic acid molecules for the transformation of host cells more efficiently.

In one embodiment the nucleic acid molecule includes a gene or recombinant nucleic acid molecule operably linked to regulatory sequences including, but not limited to, promoter sequences, terminator sequences and/or artificial ribosome binding sites (RBSs).

The regulatory sequence may be comprised of nucleic acid sequences which modulate, regulate or otherwise affect expression of other nucleic acid sequences. In one embodiment, a regulatory sequence can be in a similar or identical position and/or orientation relative to a nucleic acid sequence as observed in its natural state, e.g., in a native position and/or orientation. For example, a gene of interest can be included in a recombinant nucleic acid molecule or recombinant vector operably linked to a regulatory sequence which accompanies or is adjacent to the gene of interest in the natural host cell, or can be adjacent to a different gene in the natural host cell, or can be operably linked to a regulatory sequence from another organism. Regulatory sequences operably linked to a gene can be from other bacterial regulatory sequences, bacteriophage regulatory sequences and the like.

In one embodiment, a regulatory sequence is a sequence which has been modified, mutated, substituted, derivated, deleted, including sequences which are chemically synthesized. Preferably, regulatory sequences include promoters, enhancers, termination signals, anti-termination signals and other expression control elements that, for example, serve as sequences to which repressors or inducers bind or serve as or encode binding sites for transcriptional and/or translational regulatory polypeptides, for example, in the transcribed mRNA (see Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989). Regulatory sequences include promoters directing constitutive expression of a nucleotide sequence in a host cell, promoters directing inducible expression of a nucleotide sequence in a host cell and promoters which attenuate or repress expression of a nucleotide sequence in a host cell. Regulating expression of a gene of interest also can be done by removing or deleting regulatory sequences. For example, sequences involved in the negative regulation of transcription can be removed such that expression of a gene of interest is enhanced. Preferably, promoters include native promoters, surrogate promoters and/or bacteriophage promoters.

In one embodiment, a promoter is associated with a biochemical housekeeping gene or a promoter associated with an ethanologenic pathway. In another embodiment, a promoter is a bacteriophage promoter. Other promoters include tef (the translational elongation factor (TEF) promoter) which promotes high level expression in Bacillus (e.g. Bacillus subtilis). Additional advantageous promoters, for example, for use in Gram positive microorganisms include, but are not limited to, the amyE promoter or phage SP02 promoters. Additional advantageous promoters, for example, for use in Gram negative microorganisms include, but are not limited to tac, trp, tet, trp-tet, lpp, lac, lpp-lac, laclq, T7, T5, T3, gal, trc, ara, SP6, λ-p_{R} or λ-p_{L}. A preferred promoter for use in Gram negative microorganisms is λ phage cI constitutive promoter.

In another embodiment, a recombinant nucleic acid molecule includes a transcription terminator sequence or sequences. Typically, terminator sequences refer to the regulatory sequences which serve to terminate transcription of a gene. Terminator sequences (or tandem transcription terminators) can further serve to stabilize mRNA (e.g., by adding structure to mRNA), for example, against nucleases.

In another embodiment, a recombinant nucleic acid molecule or recombinant vector has sequences allowing for detection of the vector containing sequences (i.e., detectable and/or selectable markers), for example, sequences that overcome auxotrophic mutations, for example, ura3 or ilvE, fluorescent markers, and/or calorimetric markers (e.g., lacZ/β-galactosidase), and/or antibiotic resistance genes (e.g., htk, bla or tet).

Exemplary sequences are found in Table 3. In a further embodiment, the present invention provides a nucleic acid molecule and homologs, variants and derivatives of the sequences in Table 3 comprising or consisting of a sequence which is a variant of one of the sequences in Table having at least 80% identity to one of the sequences in Table 3. The nucleic acid sequence can be preferably 80%, 81%-85%, 90%-95%, 96%-98%, 99%, 99.9% or even higher identity to one of the sequences in Table 3.

The present invention also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

The nucleic acid sequence fragments display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hybridization to target sequences can be detected and optionally quantified. One of skill in the art will appreciate that the nucleic acid fragments may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of each of which is incorporated herein by reference in its entirety.

### Vectors

Also provided are vectors, including expression vectors, which comprise the above nucleic acid molecules, as described further herein. In a first embodiment, the vectors include the isolated nucleic acid molecules described above. In an alternative embodiment, the vectors include the above-described nucleic acid molecules operably linked to one or more expression control sequences.

### Examples

### Example 1

### Construction of Plasmids

The plasmids were constructed by standard molecular cloning techniques. Each comprises a ˜4 kb upstream homology region (UHR), a ˜4 kb downstream homology region (DHR), and a thermostabilized kanamycin resistance cassette in between. The UHR-DHR pair for a given plasmid correspond to the desired integration locus on the Thermosynechococcus elongatus BP-1 chromosome.

Plasmid pJB825 comprises: a 4.1 kb UHR for integration at site TS1 (Onai K et al. (2004). Natural transformation of the thermophilic cyanobacterium Thermosynechococcus elongatus BP-1: a simple and efficient method for gene transfer. Molec Genet and Genom 271:50-59), corresponding to the junction between base pairs 834231 and 834232 of the Thermosynechococcus elongatus BP-1 (JCC3) genome (GenBank NC_{—}004113); synthetic rho-independent transcriptional terminator (Nassal M et al. (1987). Structure-function studies on bacteriorhodopsin. III. Total synthesis of a gene for bacterio-opsin and its expression in Escherichia coli. J Biol Chem 262:9264-9270) designed to minimize transcription into the TS1 UHR region upon integration; λ phage a constitutive promoter (SEQ ID:3), active in both E. coli and Thermosynechococcus elongatus BP-1; coding sequence of the htk gene (kan^{htk}) encoding a highly thermostable kanamycin nucleotidyltransferase derived from plasmid pUB100 (Hoseki J et al. (1999)) (SEQ ID: 4). Directed evolution of thermostable kanamycin-resistance gene: a convenient selection marker for Thermus thermophilus. J Biochem 126:951-956; GenBank AB121443); Tn10 rho-independent transcriptional terminator (Hillen W & Schollmeier K (1983). Nucleotide sequence of the Tn10 encoded tetracycline resistance gene. Nucleic Acids Res 11:525-539) designed to minimize transcription into the TS1 downstream homology region (DHR) region upon integration; and 4.1 kb DHR for integration at site TS1. The sequence of plasmid pJB825 is disclosed as SEQ ID: 1 in Table 3.

Plasmid pJB826 comprises 4.6 kb UHR for integration at site TS4 (Onai K et al. (2004). Natural transformation of the thermophilic cyanobacterium Thermosynechococcus elongatus BP-1: a simple and efficient method for gene transfer. Molec Genet and Genom 271:50-59), corresponding to the junction between base pairs 483708 and 483709 of the Thermosynechococcus elongatus BP-1 genome (GenBank NC_{—}004113); synthetic rho-independent transcriptional terminator (Nassal M et al. (1987). Structure-function studies on bacteriorhodopsin. III. Total synthesis of a gene for bacterio-opsin and its expression in Escherichia coli. J Biol Chem 262:9264-9270) designed to minimize transcription into the TS1 UHR region upon integration; λ phage a constitutive promoter, active in both E. coli and Thermosynechococcus elongatus BP-1; coding sequence of the htk gene (kan^{htk}) encoding a highly thermostable kanamycin nucleotidyltransferase derived from plasmid pUB100 (Hoseki J et al. (1999). Directed evolution of thermostable kanamycin-resistance gene: a convenient selection marker for Thermus thermophilus. J Biochem 126:951-956; GenBank AB121443); Tn10 rho-independent transcriptional terminator (Hillen W & Schollmeier K (1983). Nucleotide sequence of the Tn10 encoded tetracycline resistance gene. Nucleic Acids Res 11:525-539) designed to minimize transcription into the TS4 DHR region upon integration; and a 4.1 kb DHR for integration at site TS4. The sequence of plasmid pJB826 is disclosed as SEQ ID: 2 in Table 3.

### Example 2

### Transformation of Host Cell with Plasmids

Thermosynechococcus elongatus BP-1 was transformed with pJB825 and pJB826 using the following protocol. 400 ml Thermosynechococcus elongatus BP-1 in B-HEPES medium was grown in a 2.8 l Fernbach flask to an OD₇₃₀ of 1.0 in an Infors Multritron II shaking photoincubator (55° C.; 3.5% CO₂; 150 rpm). For each transformation, 50 ml cell culture was pelleted by centrifugation for 20 min (22° C.; 6000 rpm). After removing the supernatant, the cell pellet was resuspended in 500 μl B-HEPES and transferred to a 15 ml Falcon tube. To each 500 μl Thermosynechococcus elongatus BP-1 cell suspension (OD₇₃₀ of ˜100), 25 μg undigested pJB825/pJB826 (or no DNA) was added, having been isolated from E. coli NEB 5-alpha (New England Biolabs) using a QIAprep Spin Miniprep Kit (QIAGEN). The cell-DNA suspension was incubated in a New Brunswick shaking incubator (45° C.; 250 rpm) in low light (˜3 μmol photons m⁻² s¹). Following this incubation, the cell-DNA suspension was made up to 1 ml by addition of B-HEPES, mixed by gentle vortexing with 2.5 ml of molten B-HEPES 0.82% top agar solution equilibrated at 55° C., and spread out on the surface of a B-HEPES 1.5% agar plate (50 ml volume). Plates were left to sit at room temperature for 10 min to allow solidification of the top agar, after which time plates were placed in an inverted position in a Percival photoincubator and left to incubate for 24 hr (45° C.; 1% CO₂; 95% relative humidity) in low light (7-12 μmol photons m⁻² s¹). After 24 hr, the plates were underlaid with 300 μl of 10 mg/ml kanamycin so as to obtain a final kanamycin concentration of 60 μg/ml following complete diffusion in the agar. Underlaid plates were placed back in the Percival incubator and left to incubate (45° C.; 1% CO₂; 95% relative humidity; 7-12 μmol photons m⁻² s¹) for twelve days. At this time, fifteen kanamycin-resistant colonies were observed on the plate corresponding to Thermosynechococcus elongatus BP-1 transformed with pJB825, and one kanamycin-resistant colony was observed on the plate corresponding to Thermosynechococcus elongatus BP-1 transformed with pJB826. No colonies were observed on the minus DNA transformation plate.

### Example 3

### Verifying Transformation of Host Cells by Plasmids

Four putative Thermosynechococcus elongatus BP-1/pJB825 transformant colonies and the single putative Thermosynechococcus elongatus BP-1/pJB826 were grown in 6 ml B-HEPES+60 μg/ml kanamycin, along with a control colony of Thermosynechococcus elongatus BP-1 in B-HEPES, in an Infors Multritron II shaking photoincubator (45° C.; 2% CO₂; 150 rpm). Genomic DNA was isolated from 1.5 ml of each of the six cultures using the MasterPure DNA Purification Kit (Epicentre).

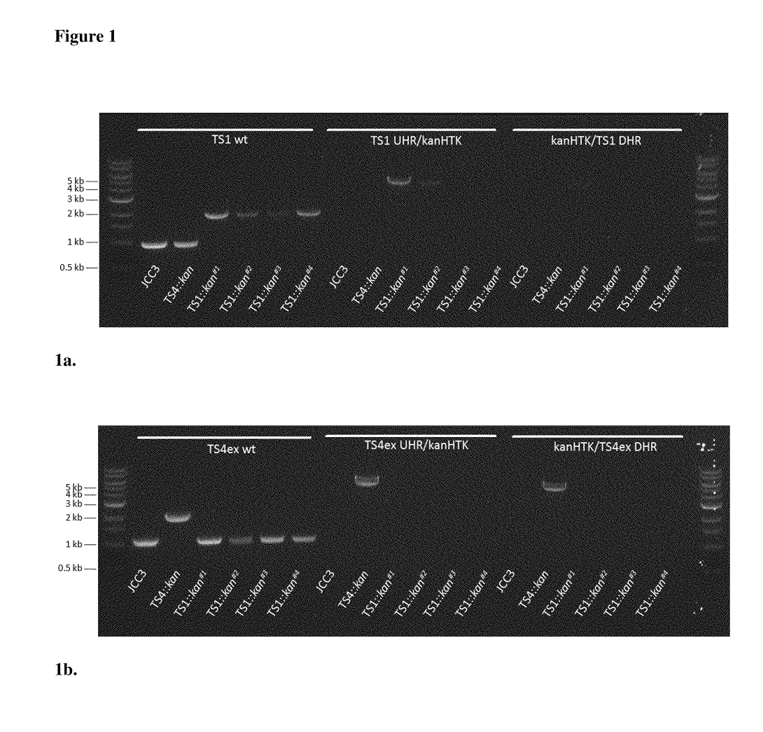

Each of the six different genomic DNA was queried by PCR using six different primer pairs (Table 1) using Phusion Hot Start High-Fidelity DNA Polymerase (New England Biolabs). For junctions involving a homology region and the kan^{htk} coding sequence, the homology region primer was selected such that it was outside the ˜4 kb homology sequence used in pJB825/pJB826. For wild-type junctions, primers were inside the UHR and DHR sequences of pJB825/pJB826. Primers are denoted in the 5′ to 3′ orientation. PCR products were electrophoresed on a 0.7% agarose/1× TBE gel versus 1 kb ladder (New England Biolabs) (FIG. 1).

TABLE 1

| | | | Expected amplicon | |
|---|---|---|---|---|
| | | | length(bp) if | |
| Junction | | | Wild- | Segregated |
| queried^{a} | Forward primer^{b} | Reverse primer^{b} | type | recombinant |
| wild- | AGATGCCAGATTCCGTTAGGTC | GATTCATCGCTTTGCAGATGTC | 958 | 1943 |
| type TS1 | | | | |
| TS1- | TCTCCAGCAATTTCTCAAGCAG | TCAGTCTGACGACCAAGAGAGC | na | 4543 |
| UHR: | | | | |
| kan^{htk} | | | | |
| kan^{htk:} | AAGCAACCAGATCTTCCTCCAG | GGGACTGCCCACCTACAGTTAC | na | 4521 |
| TS1- | | | | |
| DHR | | | | |
| wild- | GGATATTTACGATGCCCTGACC | GTGTTGAGATTCTGCACCAAGG | 1080 | 2069 |
| type TS4 | | | | |
| TS4- | GAGATTCACGTCGAACTCATGG | ATCCACCTGGATCATAAATCGG | na | 5179 |
| UHR: | | | | |
| kan^{htk} | | | | |
| kan^{htk:} | AAGCAACCAGATCTTCCTCCAG | GCAATACATCCTGCATCTGCTC | na | 4853 |
| TS4- | | | | |
| DHR | | | | |

FIG. 1 shows a 0.7% agarose gel of the 36 PCR reactions involving the six PCR primer pairs described in Table 1 and the six genomic DNA templates derived from strains JCC3, the one candidate JCC3 TS4::kan (pJB826) transformant, and the four candidate JCC3 TS1::kan transformants #1-#4 (pJB825)

The data presented in FIG. 1a indicate that the candidate segregated Thermosynechococcus elongatus BP-1 TS4::kan (pJB826) transformant is authentic as it gives a 2.1 kb band with the wild-type TS4 junction primer pair, a 5.2 kb band with the TS4-UHR: kan^{htk} junction primer pair, and 4.9 kb band with the kan^{htk}:TS4-DHR primer pair.

The data presented in FIG. 1b indicate that the candidate segregated Thermosynechococcus elongatus BP-1 TS1::kan #1 (pJB825) transformant is authentic as it gives a 2.0 kb band with the wild-type TS1 junction primer pair, a 4.5 kb band with the TS1-UHR: kan^{htk} junction primer pair, and 4.5 kb band with the kan^{htk}:TS1-DHR primer pair.

### Example 4

### Preparation of Ethanologen Constructs

Starting with plasmids pJB825 and pJB826 as described in Example 1, ethanologen constructs were prepared.

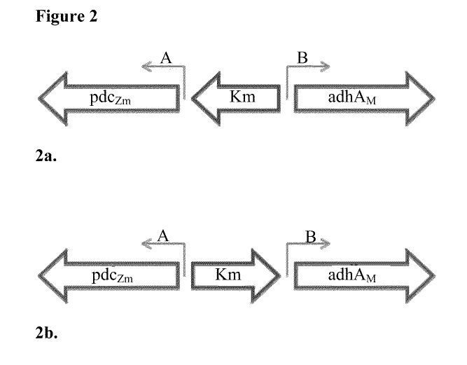

The genes for ethanol production, including pyruvate decarboxylase from Zymomonas mobilis (pdc_{Zm}) and alcohol dehydrogenase from Moorella sp. HUC22-1 (adhA_{M}), were cloned such that each gene was oriented in a divergent orientation and expressed under the control of a unique promoter. The divergent orientation means that the two genes are transcribed in opposite directions. In one configuration, expression of pdcZm and adhAM were driven by λ phage cI (“PcI”) and pEM7 and in another expression was driven by PcI and PtRNA^{Glu}. Central to the pdcZm and adhA gene was KmR, a gene conferring resistance to kanamycin. FIG. 2 shows a diagram of the pJB825 ethanologen constructs and the divergent orientation of the pyruvate decarboxylase and alcohol dehydrogenase genes. A and B are the promoters for the genes. FIG. 2a illustrates a construct where KmR is oriented in the same direction as pdc_{Zm} and FIG. 2b illustrates a construct where KmR is oriented in the same direction as adhA_{M}.

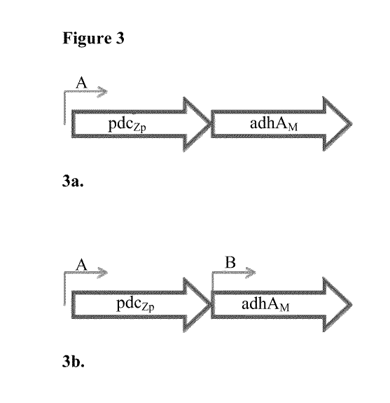

In the pJB826 ethanologen constructs, the pyruvate decarboxylase from Zymobacter palmae (pdc_{Zp}) and alcohol dehydrogenase from Moorella sp. HUC22-1 (adhA_{M}), were cloned such that the genes were in the same orientation. They were expressed either by a single promoter driving expression of both genes, or a unique promoter driving expression of each gene separately. FIG. 3 shows a diagram of pJB826 ethanologen constructs. FIG. 3a illustrates an embodiment in which both pdc_{Zp} and adhA_{M} are driven by the same promoter, A. In one embodiment, the single promoter is PaphII. FIG. 3b illustrates an embodiment in which pdc_{Zp} and adhA_{M} are driven by separate promoters, A and B. In one embodiment A is PaphII or Pcpcb and B is PcI.

### Example 5

### Production of Ethanol

JCC3 cells were grown in 800 ml B-HEPES medium in a 2-L baffled Ehrlenmeyer flask at 45 C, 100 uE, 150 rpm to an OD₇₃₀ of 1.6. The cells were then concentrated by centrifugation and resuspended in a total of 6 ml B-HEPES. Five hundred ml of concentrated JCC3 recipient cells were transferred into a 15-ml culture tube for each transformation. Transforming DNA as prepared in Example 4 (approx 60 μg in 800 μl) was added to the recipient cells and the transformation mix was incubated at 45 C in the dark for 4 hours. After 4 hours, 5 ml of B-HEPES medium was added to the transformation mix and the cultures incubated at 45 C, 100 μE at 150 rpm in an atmosphere of 2% CO₂. After 24 hrs incubation, 500 μl of overnight culture was transferred to 1.5-ml microcentrifuge tube and centrifuged for 3 minutes at 13,000 RPM. The supernatant was transferred to a clean microcentrifuge tube. Ethanol and acetaldehyde concentrations were determined by GC-FID. The resulting concentrations of ethanol and acetaldehyde are show in Table 2.

TABLE 2

| | Acetal- | |
|---|---|---|
| | dehyde | Ethanol |
| Transforming DNA | (mg/L) | (mg/L) |
| No DNA | 0.35 | 7.3 |
| pJB826 (vector-only control) | 0.2 | 77.7 |
| pJB825_PEM7_pdcZm_Km_PcI_adhAM | 1.28 | 13214.8 |
| (SEQ ID NO: 6) | | |
| pJB825_PcI_pdcZm_Km_PEM7_adhAM | 3.14 | 15628.1 |
| (SEQ ID NO: 5) | | |
| pJB825_PtRNAglu_pdcZm_Km_PcI_adhAM | 3.31 | 15090.9 |
| (SEQ ID NO: 8) | | |
| pJB825_PcI_pdcZm_Km_PtRNAglu_adhAM | 3.46 | 15752.1 |
| (SEQ ID NO: 7) | | |
| pJB826_PaphII_pdcZp_PcI_adhAM | 2.39 | 1729.5 |
| (SEQ ID NO: 9) | | |
| pJB826_Pcpcb_pdcZp_PcI_adhAM | 0.77 | 1317.1 |
| (SEQ ID NO: 10) | | |
| pJB826_PaphII_pdcZp_adhAM | 0.84 | 2091.1 |
| (SEQ ID NO: 11) | | |

TABLE 3
Informal Sequence Listing

SEQ ID: 1
TGGGAGTCAATAAACCCGATGTGCGTTGGATTTGCCACTACCAGCCGC
CCCTGCAACTCAGTGAATATCTCCAAGAGGTGGGACGCGCTGGGCGAG
ATGGCGAAGCGGCACAGGCCCTGGTTTTGGTGAGCGATCGCTGGGGCT
TGGATCGCGAAGATCAACAGCGTTGGTCTTTTTTTCAGCACCAAAGTC
AAGACACCTACAATCGCGCCATGGCACTTCAGACGCAGCTGCCCCTCC
AGGGTAATCTGCAGCAACTGCGGCAACACTTTCCTGAAGTGGAATTGA
CCCTGGCATTACTGCATCAACAGGGGGCCCTCCGCTGGCAAGATCCCT
TTCACTATTGCCGTCAACCCTTGGCACAGGTGCCACCCCCACCCAAAG
ACCCTCAAGAACAGTTGATGCAAAAGTTCCTCTATCACCGGGGCTGCC
GCTGGCAGTTTCTCCTCCAAGCCTTTGGTTTTGCCACTGAGGCAAGGG
GATTCCACTGTGGCCATTGCGATCGCTGTCGGCCGCCGCACCGCTCCC
GCAAAATACCGTAAATTGCCAGCGCTGTATCACTGGAATATTGGGTAC
ACTGGCACATAGAACGGTCGCTTTACCATTGGTAGGCAAAAGTTTCTC
AGCAGTCATTCTGTTGCCGCAAGGTAGGGGTTGCAGGCATGGGGCTAC
TACAAGTTGAGGAAATTCGCGAAGCACTTCAAGATGTGCTTTCAGAAC
ACGCCCTTGTTGTGCAAGTTAATCAGTTTCGCAACCAATTAAACATTA
TTTTGAACAAGCCCCCCGGCACCGTTGCCCATTATTCTGCCCTAGCGG
ATTTTCTCAAGTCGCGCTTGGGACAGTTTCATCTCAATGATATTGACC
GCATTAAAATAATTGGCCGCATACAGGGTTCGCCTAAACCCGATTGGG
AAGAGGTCATTGATCTACGTCCCCCCAACCCAGCCCTAGCTGCCCCTG
TGTATGCTTCTTCTGCCCCGTGGGTGGTGGCGATCGCTGCTGGCTTTG
TCAGTTTACTGGTGATCTTTAGCTATCACCTTGGTCAGTAGCAGCAAC
AGCAACGGCTGTAGCCGTTGATCGAAGGTTCCTTTGGTCAAAAGGGCG
TCGTGATGACGGACTTTAAGTGGCACATTGAGGGTGGTACAGGGTTTA
TTGTCGGGGTTCTTAAAAACTACAGTAAAGGGTATTTTCGCTTAGTTC
AGGCGGACTTTGAACTCTTTGACCAAGGCGGTCAGCAAGTTGGGACAG
TGGCGGTACAGGTTTATGGTCTTGGCCCTGAGGAAACATGGCAATTCC
GTGAACTGATAGCCAATCATCAGGCAGTGCGAGCACGGCTGGTAAAAT
TACAGTCATTCAATTAAGGTTTTTCTAATGTTTAGGTTTCCCCAGCAG
GGAGCGACACCGCTTGCTATGGCACACCTTAAAGCCCTGATCTTTGAT
GTCGATGGCACCTTAGCAGATACGGAGCGGGATGGCCATCGTATCGCC
TTCAACAAGGCCTTTGCCGCCGCTGGTCTAGATTGGGAATGGGACATT
CCCCTCTATGGTCAACTCCTGGCGGTGGCTGGGGGCAAGGAGCGGATC
CGGTATTACCTTGAGTGCTTTCGTCCCGATTGGCCACGTCCCCAAAAT
TTGGATGCTCTGATTGCCGATTTACACAAGGCCAAGACCCGCTATTAT
ACCGAGCTATTGGCGGCAGGGGCTATTCCCCTGCGGCCGGGGGTGAAA
CGGCTCCTCACTGAAGCCCGGGAAGCAGGATTACGTTTGGCGATCGCC
ACCACGACCACCCCTGCCAATGTCACCGCACTCCTTGAAAATGCCCTC
GCTCCTGATGGCGTCAGTTGGTTTGAGATAATTGCTGCCGGGGATGTA
GTTCCAGCCAAGAAACCCGCGCCCGACATTTACTTCTACACGCTTGAA
AAGATGCGCCTCTCACCCCAAGAGTGCCTTGCCTTTGAGGATTCCGCC
AATGGGATTCAGGCGGCCACTGCCAGTCACCTAGCGACCATTATCACG
ATTACCGACTACACCAAGGATCATGATTTTCGTGATGCAGCGCTGGTC
TTGGATTGCTTAGGGGAACCGGACTACCCCTTTCAGGTTCTGCGCGGT
GAGGTGGGTTGGACAACCTATGTGGATGTCCCCCTATTGCGATCGCTG
CACCAGCAGTGGACAAGCACGTTGAGTCAGGGATAATTTTCTGGCCGC
AGCGTTTTACATTGAATATGACCCCCTTAGTCTGAGGATCAAGGAACA
TAATGTACACGATTGATTTAATTCTGCGTCATGTCCCCATGCCCGTCA
GCATTGAACGCAAGGAAAGTGCAGCAGCGATGGCAGTCTATCAGCAAA
TCAGCAGGCCATGGCCAGTGGTACTCCAACTTTCCTCGAACTGACGTG
CGATCGCCAAGTGGGCAAGAAGTTAACGGTGCTCACCTCAGAAATTGT
CGCCGTGCAAATGGCGGATAAGGATGCCCCCTCCAGTACTATCAGTCG
TGGGGGATTCTTTGCTCAATTAGTGCAGCAAACCAGCAACTGAGGGAA
AATGCCTCAATAAAGTTGAGTTTTTCTTGGCAATGCTGATTCTTTGCC
GTTAGGATACTAAGCAGACCGATCCGTAGGGGAACGTGAAGCAAATCC
TCCCCGTCTGAAAGTCAGGTATCTCTGGTGTGTCGTAATAGGGTTGTC
TATGGTGCAGCGTTTCCTGCCGGTTCTGATTTTGTTGGGGTGTAGTTT
TGGTCTTGCGACCCCTGCCCTTGTGCGTGCCCAAGCCAATCAGGGCTT
TACGTTTACTTGGGGTGAGGGGCCGAGTGGCCGACAGCAGTTGCAATA
CCACTTAGATAACGGCACCCCCGGTTTTATGGGCGATCGCTATTGGCT
GCGGCTGGGTCAGCAGAAAGTGGCCATCAATCGCATTAACATTACCTA
TCCCGACTACTACAACGGTATTATTGATCCCAAAGGCATTGAGGTGCG
CATCGGTGGCGATCGCGGCAATCGCTTCTTCCAATTTCGCCGTGACCC
CGGCACCAAAATTCAATTGGCGGAAGTCTCCGTTGATCGCGATAACCG
CGTGATTGATATTGTGCCGGCTGAGGTGATTCCCGCCGGAACACCGGT
GCAAGTTATTCTCAATAATGTGCGCAACCCTAACAATGGCGGCATGTA
CTATTTCAATGCCCGCATTGGCTCCCCTGGAGATATTCCCCTCATGCG
CTACGTTGGCACCTGGATTCTCAGCATTGCCAATAACTAAAACCCGTC
AAACTCGAGCATTGGTGAGCGGGTTAGCCATTTCTAACTATTGCGGGG
CGATCGCCCTAGACTAGTTTTTTGTCTATTATTGCCGGTTCACTCTTT
ACACCAGATGCCAGATTCCGTTAGGTCTTCATTCCCCTCCATTTCTCC
TCTGCTCACGCCTCTGATGTACCGCCTCGTGGGGGACGTTGTCCTGCG
GCGCTATTTTCGTACCCTTGAGGTGCAAGGGCAGGAGCGGGTGCCCCA
AAGGGGTCCAGTGATCTTGGCCCCCACCCACCGTTCCCGCTGGGATGC
GCTGATTATTCCCTATGTCACTGGGCGGCGGGTGAGTGGGCGCGACCT
CTACTACATGGTGTCCCACGATGAGATGTTGGGACTACAGGGCTGGGT
GATTGCTCAGTGTGGCGGTTTTCCCGTCAATACCCAAGCGCCTTCGGT
GAGTGCGTTGCGTACGGGTGTGGAACTGCTCCGGCAGGGGCAAGCCTT
GGTGGTGTTCCCTGAGGGGAATATCTTTCGCGATCGCCAGATTCATCC
CCTCAAGCCGGGGTTGGCTCGCTTAGCCCTTCAGGCGGCCCAGCGCTG
TGAACAAGCAATCCAGATTCTGCCAATTTTACTCGATTATGCCCAGCC
CTACCCACAGTGGGGAAGTGCGGTCAAGGTAATCATTGGGGCTCCCTT
GAGTACCGACAATTACGATGCCAGCCGGCCAAAAAGTGCTGCCCAACA
ACTGACCAGTGATCTCTTTAGAAGACTTCAGCAGCTCCAAGGGGGGCG
ATCGCCCCTGTGTTTTGCTTAGACCTCAAACTTCCATCCCCGCGGCCG
CAAAAAAAACGGGCCGGCGTATTATCGCCGGCCCGAGTAACACCGTGC
GTGTTGACTATTTTACCTCTGGCGGTGATAATGGTTGCAGGATCCTTT
TGCTGGAGGAAAACCATATGAAAGGACCAATAATAATGACTAGAGAAG
AAAGAATGAAGATTGTTCATGAAATTAAGGAACGAATATTGGATAAAT
ATGGGGATGATGTTAAGGCAATTGGTGTTTATGGCTCTCTTGGTCGTC
AGACTGATGGGCCCTATTCGGATATTGAGATGATGTGTGTTCTGTCAA
CAGAGGGAGTAGAGTTCAGCTATGAATGGACAACCGGTGAGTGGAAGG
CGGAAGTGAATTTTTATAGCGAAGAGATTCTACTAGATTATGCATCTC
GGGTGGAACCGGATTGGCCGCTTACACATGGTCGATTTTTCTCTATTT
TGCCGATTTATGATCCAGGTGGATACTTTGAGAAAGTGTACCAAACTG
CTAAATCGGTAGAAGCCCAAAAGTTCCACGATGCGATCTGTGCCCTTA
TCGTAGAAGAGCTGTTTGAATATGCAGGCAAATGGCGTAATATTCGTG
TGCAAGGACCGACAACATTTCTACCATCCTTGACTGTACAGGTGGCAA
TGGCAGGTGCCATGTTGATTGGTCTGCATCATCGCATCTGTTATACGA
CGAGCGCTTCGGTCTTAACTGAAGCAGTTAAGCAACCAGATCTTCCTC
CAGGTTATGTCCAACTGTGCCAGCTCGTAATGTCTGGTCAACTTTCCG
ACCCTGAGAAACTTCTGGAATCGCTAGAGAATTTCTGGAATGGGGTTC
AGGAGTGGGCGGAACGACACGGATATATAGTGGATGTGTCAAAACGCA
TACCATTTTGATGTCTAACCCCCTTCCTTGCCCACAGCTTCGTCGATG
GCGCGAAATTTCGGGTAAATATAATGACCCTCTTGATAACCCAAGAGG
GCATTTTTTAGGCGCGCCCTAAGCGTCCGTAGGCACAATTAAGGCTTC
AAATTGTTGGCGAAGCTGCTCAGTCACTTCCTTGACGGCTTGCCGTGC
CCCTTGGCGATCGCGCCGGTACAGAGGCCAATAGCTCTCTAAATTGAG
AGGGTCGCCGACACTGAGGCGCACCTGCCGCAAACCCACCAAACGATT
GAGATTCGAGCTTTTTCCCTCTAGCCAATCAAATGTGCGCCAGAGAAT
CAGCGCGACATCTGCAAAGCGATGAATCGTGAATTTCTCACGGATATA
GCTACCCGTAATTGAGGTAAATCGCTCCGCAAGACGCATATGACGCAA
TCGCACATTGGCTTCCTCGGCCAACCAATCGGCTAGGCAGCGCTCTAC
GGCCGAAAGTTGTGCCAAATCACTGCGAAACATCCGTTCCCAAGCAGC
CTGTTCAATGCGTCGGCAGCGACTCACAAAATCGGCACTGGGCTTCAG
ACCAAAGTAGGACTCTGCCACCACAAGGGCGCTGTTGAGGAGGCGCTG
AATTCGCGCTGCCAATTTAGCATTGGCAGAGTCAAAGGGGGGCAGTTC
GGGAAAATCTTGACCATAGGAGGTGGCATAAAAAGCCTCCAGGCGATC
CAAGAGGTGGATCGCTAAATTCAGCAGGCGGCGGTAGAGGTCGTCTGG
CTGGGTACTGTGAGAATCTGTAGGGCACCCAAGGCGGTTCTCCAGTTG
TGCCATCAGCCTTGCCATGCGCTCCCAAGAGGGCTGACTGAGGCTGTA
CTGAATGCCAATGGGAAGAATGACCACGGGGAGCGATCGCCCCGCCTT
GGCTAAATCTTCTAGACACCAAAATCCCAGTTGGGCCACCCCCGGCTC
CAAAGGTGCGACCAGTTCGTTGTGCTCATTCGTTGCTCCCTCCGGCGC
TGCCGCTAGGGGAAATCGTCCTCCGAGAAGTAGCTCCCGCGCTGAGCG
CAGGGCTTGGCTATCGAGCTTACCGCGCATGATGGAAATCCCCCCCAA
CCGTGAAAAGAGCCAACCAATCTGCGCCCCTGCCCAGAGGGGAATCCC
GCGATCGTAGAGAAAATAGCCATTTGTCGGCGGACGCAAGGGAATGCC
CAGCCGCCGTGCTGTTTGCGGCAGTAAATGCCACATCAAATAGCCCAT
CACCAACGGATCATCCGTACAGGGATGGCGAAAGGCAATGAGGAGCCG
GACCTGTCCCTGCTGAAACTGCTGGTAATAACGGGCAAGGGTCTCCAC
ATTCACCCCTTCAACCCGCTGTAGCCCAAGACCATAGCGAATGTAGAG
GGGCAGGAGTCTTGCTACTGTCCACCAGACGGGGTAGCTAAACCGCTG
GGGGAGAAAATGCAACGGCGGTTGGGCAGTTGTCACTACACTGGACAT
TAGGCAAGCTCCTCAGGGCAATGGCTAAACTGAGGCAGTGGCCAACTC
CGCAATTAACTGCTCTAACATCGGTTGATCGGCCCAATAGACAGCATT
ACAAAACTGACAGGTGGCTTCTGCCTTTGCCTCTGTGGCTAGGATATC
TCTTAATTCTGCCTCCCCTAGGAGCTTGAGTGCCGCTAACATCCGTTC
ATGGGAACAGCCACAGTGGAAGCGCACCATTTGCCGTTGGGGCAAGAT
TTGTAAATCCATATCCCCTAAGAGTTCCTGAAAGATATCTGGCAGTGT
CCGCCCTGCCTGTAGCAGTGGTGTAAAGCCCTTAAGATTGGCCACCCG
TTGTTCAAGGGTCGCGATCAGGTGTTCATCATTGGCCGCTTTGGGTAG
CACCTGTAACATCAACCCACCGGCGGCAGTCACCCCGGACTCTTCGAC
AAAAACACCCAACATCAGGGCGGAGGGGGTTTGCTCTGAGGTGGCGAG
GTAGTAGGTGATGTCTTCTGCAATTTCGCCGGAGACTAGCTCCACCGT
GCTGGAATAGGGGTAGCCGTAGCCAAGATCGTGGATGACGTAGAGATA
TCCCTGATGGCCCACCGCTGCCCCCACATCGAGTTTGCCCTTGGCATT
GGGGGGCAGTTCAACACTGGGGTACTGCACATAGCCGCGAACTGTGCC
ATCGGCACCAGCATCGGCAAAAATGGTTCCTAGGGGACCGTTGCCCTG
AATGCGCACATTCACCCGTGCTTGGGGCTGTTTGAAACTGGAGGCAAG
GATTAAGCCTGCGGCCATGGTTCGTCCCAAGGCCGCTGTGGCCACGTA
GGACAGTTGGTGACGTTTGCGGGCTTCATCAGTGAGTTGAGTGGTAAT
CACACCTACGGCCCGGATGCCTTCGGCAGCGGCAGTTGCTCGCAACAG
AAAATCGGCCATGTTCAACCTACGAAATGTTTTGTTACATTTAGTGTG
ACATACTCCCACCGCTGACCAGGGCACAATGGGGCAAAAAACCATCAA
TCCTGCCTTTGGTGACCGATCCAGTACAGCCAGCCAGGGCTTAAGACT
GGGAAGACCCCTAGCACTGGGGCTAGAAAATTGGCGATGATAGGCAAG
CAATAGTCATTCAGCGTCCAGTCATTCCGCCTATGGCCATGCCCCTCA
CTGTCTTGCCTGCCACAACTGTTTTGACAGAAGCGACTCAATTGCCCC
AGGGCGGCTTGATTACGGAGATTCCGACGCTGGCGATCGCCCACCGTT
TGGCCCAGCAGTTGCGCCGCCATTGGCCCCTAGAGACCCCCTTAACGC
TGATTGATGCGCAATACCAGAGTATCCCCCTGACCCTTGGGGAATTGG
CCGAGCTCACCGATGCCAACTGTCCTTTACAGCTCTATGTGCCGCCCC
CCTTGCCAGAGGCCTTGACGCAATTTCAACGCCTGATGGATGTGGTTC
GAGAGCTGCGCCATCCGGAGCGTGGCTGTCCTTGGGATTTGCAGCAAA
CCCCAACCAGTCTCATTCCCTATGTCCTTGAGGAAGCCTATGAAGTGG
TACATGCCCTGCAGGAGGGAGATGCGGGGGCGATCGCCGAAGAATTGG
GAGACCTGTTGCTTCAAGTTGTTCTCCAGAGCCAACTTGCCCAAGAAG
CCGGCCAATTTACCCTTGCTCAAGTCATTCAAAGGATTACCGATAAAC
TCATCCGCCGCCATCCCCACGTCTTTGGTGAAGTGGCACTCACCACTG
CTCAAGAGGTGCGCGACCAATGGGAGCAAATCAAAGCGGCTGAAAAAG
GCACCGAACTCCCCCTGAGTCAAACGCTGCAACGTTACGCACGCACCC
TCCCACCCCTGATGGCCGGCATGAAAATTGGTGAGCGAGCCAGTCGCG
CTGGCCTCGATTGGCCGACGATTAGTGGTGCATGGGAGAAATTTTACG
AGGAACTGGCGGAGTTTCAGGAGGCCCTTCTGCAAGGGAATGCTGAGC
AACAGGCAGCGGAATTAGGAGACCTGCTCTTCAGTGTGATTAACCTTG
CCCGCTGGTGCCAACTGGATCCTGTTAATGCCCTGCAACAAACCTACC
AACGCTTTATTCAACGCTTGGCCTGTATTGAGGCAGTCATCGATCGCC
CCCTTGAGACGTACACCCTAGAAGAACTAGAAGCCCTCTGGCAACAGG
CCAAAGTACAGTTAGCCACCGACAGCGAGGCAACCCCTATGGAGACTG
AGGAAGAGGCCTAGTCCGCTGCGGCCCTTGCCACCTTCAGTTCATCGA
GATTCCACAGGGGGCCCCCCAGCGCCGTGGGCTTGGCGCCAATGACAT
GATTGCGAAAAGCTGTAAGGGAGAGGGGATTCACGAGGTAAATAAAGG
GGAGATATTCCTGAGCTAGTCGTTGGGCTTCCGCATAAATTTGCTGCC
GTCGTTCCAGATTGAGCTCCTGGGCACCTTGGACATACAGGTCACTGA
TGCGCTGCTCCCAGTCAGCGACGACTCGACCCGTAATGGGTGGTTGAT
TCGGTGACGGTTGCTGATTGAATGTATGCAAAAGGCCATCCACACGCC
AGATATTGGCACCGCTATTGGGTTCATTGCCCCCCCCAGTAAAGCCGA
GGATATGGGCTTCCCACTCTAGGGAATTGGAGAGACGATCCACGAGGG
TACCAAAGGCCAAAAATTGCAGATCCACCTGCATGCCGATCGCCCCTA
GGTCCTGCTGAACTTGCGTCG
SEQ ID: 2
TCCGCGGGAGGTGTAATGCCGATGGCCCCCTTGCGGAAAACCTATGTT
CTCAAGCTATACGTTGCCGGTAACACACCCAACTCGGTGCGTGCCCTA
AAAACTCTCAATAACATTCTTGAAAAAGAATTTAAGGGAGTCTATGCA
CTCAAAGTAATCGATGTCCTCAAAAATCCGCAACTGGCTGAGGAAGAT
AAAATTTTGGCCACGCCTACCCTTGCCAAAGTCCTACCGCCCCCTGTG
CGCCGGATTATTGGGGACTTGTCGAATCGTGAGAAGGTGCTCATTGGC
TTAGATCTCTTGTATGAAGAGATTGGTGACCAAGCCGAGGATGACTTA
GGCTTGGAATAGGCACAGTCCTTAGAGACTCTCAGTTTAGAATAGCTT
CTTGGAATTTTTGCGCAATACCGAATCTAAAAATCTTCTATGACAAAC
CTACCGGAACATCAGTCTAGTCCAACGGAGCAGTCCTCTGCGGAAGTC
AAGAAAATCCCGACGATGATTGAGGGCTTTGACGATATCAGTCATGGG
GGACTTCCCCAAGGACGCACCACCTTAGTCAGCGGCACTTCAGGCACA
GGGAAGACCCTTTTTGCAGTTCAGTTTCTCTACAATGGCATTACCATT
TTTAATGAGCCAGGTATATTTGTTACATTTGAAGAATCCCCCCAAGAT
ATTATCAAAAACGCCCTCAGTTTTGGCTGGAACCTGCAAAGTCTGATT
GATCAAGGCAAGCTATTTATCCTGGATGCTTCTCCGGATCCCGATGGC
CAAGAGGTGGCTGGTGACTTTGACTTATCTGCTCTGATTGAGCGCATT
CAGTATGCCATTCGCAAATACAAAGCAACCCGGGTCTCCATTGATTCG
GTCACAGCAGTGTTCCAGCAATACGATGCGGCCTCCGTGGTGCGGCGG
GAAATTTTTCGCTTGGCTTTTCGCCTCAAGCAACTGGGCGTGACCACG
ATTATGACCACTGAGCGGGTAGATGAATACGGCCCTGTGGCGCGTTTT
GGTGTTGAGGAGTTTGTCTCCGACAATGTGGTCATTTTGCGGAATGTT
CTCGAGGGAGAAAGGCGGCGGCGCACGGTCGAAATTCTCAAGCTGCGG
GGCACCACCCACATGAAGGGGGAATATCCCTTTACGATCAACAATGGT
ATTAACATCTTCCCGTTGGGGGCCATGCGCTTGACTCAGCGCTCATCG
AATGTGCGGGTGTCTTCAGGGGTCAAGACCCTCGACGAGATGTGTGGC
GGTGGCTTCTTCAAGGATTCAATTATTTTGGCCACGGGCGCTACGGGT
ACTGGCAAGACGCTCTTGGTCAGTAAATTCTTGGAGACGGGCTGCCAA
CAGGGAGAACGAGCCCTGCTGTTTGCCTATGAAGAATCGCGGGCGCAG
TTGTCGCGCAATGCCTCCTCTTGGGGTATTGATTTTGAGGAGTTAGAA
CGGCGCGGTTTGTTGCGGATTATTTGTGCCTATCCAGAGTCAGCGGGG
CTTGAGGATCACCTGCAAATTATCAAGTCGGAGATTGCGGACTTTAAG
CCCTCACGGGTGGCGATTGACTCTTTGTCTGCGTTGGCGCGGGGGGTG
AGTAACAATGCCTTCCGGCAGTTTGTAATCGGGGTTACTGGATTTGCC
AAACAGGAGGAAATCACTGGCTTTTTCACCAACACGACGGATCAGTTT
ATGGGGTCCAACTCGATTACCGAGTCCCATATCTCCACAATTACAGAC
ACCATTTTGCTGTTGCAGTACGTGGAAATCCGCGGTGAGATGTCGCGG
GCAATTAATGTCTTTAAGATGCGTGGCTCTTGGCACGACAAGGGGATT
CGGGAGTATGTGATCACTGAGAAGGGGGCAGAAATCCGCGATTCCTTC
CGCAACTTTGAGGGGATTATTAGCGGTACCCCCACCCGCATTTCCGTG
GACGAAAAAACAGAGCTGGCGCGAATTGCCAAGGGGATGCAGGATCTA
GAGAGCGAGTAGCCCCATGCAGTTAAACCAAGTTATTGTGGTGCACAA
GGCGGGCGATCGCCAGAGCAAGGAATGGGCAGATCGTGCCTCCCGTCA
ACTACAACAGCGTGGCGCCAATGTGCTGGTAGGGCCTAGTGGGCCTAA
GGACAACCCTTACCCCGTCTTTATGGCCTCTGTGACAGAGCCGATTGA
TCTCGCCGTTGTTCTGGGGGGCGATGGCACCTCCTTAGCAGCGGCACG
CCATCTCGCAGCGGCTGGGGTTCCAATTTTAGCGGTGAATGTGGGGGG
GCATTTGGGGTTTTTGACGGAGCCCTTGGAGTTGTTTCGCGATATGGA
GGCGGTTTGGGATCGCCTGGAGCGGGATGAGTACGCGATGCAACAGCG
GATGATGCTGCAAGCCCAGGTTTTTGAAGGGTCAAAGGCTCATCCGGA
AGCGGTGGGCGATCGCTACTATGCCCTGAATGAAATGTGCATTAAGCC
GGCCTCTGCTGATCGCATGATCACCGCCATCCTCGAGATGGAAATTGA
TGGCGATGTTGTGGATCAGTACCAAGGGGATGGGTTGCTGGTGGCCAC
GCCCACTGGCTCTACTTGCTATACGGTCGCCGCCAATGGCCCCATTTT
GCATCCAGGGATGGAAGCCCTGGTGGTGACACCCATTTGTCCTTTGAG
TCTCTCTAGCCGCCCCATTGTCTTGCCTGCGCGCTCCTCAGTCAGCAT
TTGGCCCTTGGAGGATCACAGTCTCAATACCAAGCTGTGGATGGATGG
TGTCCTGGCCACCTCCATTTGGCCAGGACAGCGGGTACAGGTGACAAT
GGCCGATTGTCAAGCTCGCTTTATCATCCTGCGGGATCACTACTCCTT
TTATCAAACCCTACGGGAGAAGTTAGCCTGGGCAGGGGCACGGATTCC
CTATCACAACAATCACCGCAATTAGATCACAACCGCCCCTCCAGAAGG
TCTTTATAATTGGGGCATTCCTCACTAAACCCTTGCTATGATTCTCAG
TCCCTTTGAACGCGCCGTTCTTGGCCAAGAGGCGGAAGCCCTGGTTGA
TCAGTTGTTAGAAATTGGGATTTCCCTCTCTGCCAGTCAATCCCTAGA
GGAATTGCTGCATCTGATTCTCACGAAAAGTCGCCAAATCACTGCTAG
CGATGCTGGCACGATTTTTCTAGTTCAGCGGGAACGGGCAGTGCTGGA
ATTCAAGGCAGCTCAAAACGATAGCGTCACCCTTCCTGAGCAAGTGCA
GGACTATACCATACCCCTCACCGCCGATAGCTTGGTGGGCTATGCCGC
TCTCACGGGGGAATCCCTAAATATTGCCGATGTGTATGCCCTCAAGGG
GAGCGAGATGTACCAGTTCAATCGCTCTTTTGATGAAGCCCTCCACTA
TCGAACCTGTTCGGTGCTGGTGGTGCCGATGCAAAATATTAGCGGTGA
GGTGATTGGCGTTCTGCAACTGATTAACCGCAAGCGATCGCCCGATAC
CCGGCTGAGACCAGAAACCAGTGTGGCCCTCACCCAGCCCTATAGTCC
TTGGGAAGAACATATTGTGCGATCGCTGGCCAGCCAAGCGGCGGTGAT
TATTGAGCGCAATCATCTGCTCGAGAGTATTGAACAGCTCTTTGAGGG
ATTTATTACCGCTTCAGTTCAAGCCATTGAGACGCGAGATCCAGTCAC
CGCAGGGCATTCGGAACGGGTGGCAGCGCTGACGGTGCGCCTTGCTGA
GATCACCAATGCCACCTCTAGGGGAGTCTTTCGCGATGTTTTCTTTAG
CGATCGCCAGCTCCAGGAAATCCGCTATGCTGCTCTGCTCCACGATTT
TGGCAAGGTGGGCGTGCCGGAGGCAATTCTCAACAAGCAAAAGAAATT
CTACCCCGAACAGCTAGAGGTGATTCGCCAGCGCTTTGCCCTCGTCCG
CCGCACCCTTGAAATGGAAACGGCTCAAGCCAAAGTCAATTATTTACT
CTCCCATCCCCATCAGCCCCATACCCCACAACAGCGGTGTCAGTCCTG
TACTTTTTTACGAGACCTCGATCAGCAACTCCAGCAACAACTGCACAC
CCTAGAGGCCTACTGGCAGCTAATTGAGCAGGCCAATGAGCCGCAAAT
TCTTGAGGAGGAACCCCTGGCTCAGCTTCAGGAATTGACCCAGTTTTA
TTACCGCGGCACTGATGGGGAACTCCATCCCCTGATCACGGCCAGCGA
ACTGGAGCAACTCTTGGTGCGGCGGGGCAATCTCACCCAAGGGGAGCG
GCGCATGATTGAAGCCCACGTCACCTATACCTACGAGTTTCTCTCGCG
CATTCCTTGGACACCCCACCTGAAGAATGTGCCGATCATTGCCTATGG
TCACCATGAGCGCTTAAATGGCAGTGGCTACCCCCGCGGTATTGGTGC
CGCCGAAATTCCCCTACAAACCCAAATGCTGGCGATCGCGGATATTTA
CGATGCCCTGACCGCCAAGGATCGCCCCTACAAAAAGAGCCTACCTGT
GGATAGGGCCCTAGGGATTTTGTGGCAGGAGGCTAGGGAATTTAAGAT
TAATCCTGATCTGGTGGAACTCTTTGAGCAGCAGGAGGTCTTTCGGGT
GCTGGGGCACCAGCGCTAGGCGGCCGCAAAAAAAACGGGCCGGCGTAT
TATCGCCGGCCCGAGTAACACCGTGCGTGTTGACTATTTTACCTCTGG
CGGTGATAATGGTTGCAGGATCCTTTTGCTGGAGGAAAACCATATGAA
AGGACCAATAATAATGACTAGAGAAGAAAGAATGAAGATTGTTCATGA
AATTAAGGAACGAATATTGGATAAATATGGGGATGATGTTAAGGCAAT
TGGTGTTTATGGCTCTCTTGGTCGTCAGACTGATGGGCCCTATTCGGA
TATTGAGATGATGTGTGTTCTGTCAACAGAGGGAGTAGAGTTCAGCTA
TGAATGGACAACCGGTGAGTGGAAGGCGGAAGTGAATTTTTATAGCGA
AGAGATTCTACTAGATTATGCATCTCGGGTGGAACCGGATTGGCCGCT
TACACATGGTCGATTTTTCTCTATTTTGCCGATTTATGATCCAGGTGG
ATACTTTGAGAAAGTGTACCAAACTGCTAAATCGGTAGAAGCCCAAAA
GTTCCACGATGCGATCTGTGCCCTTATCGTAGAAGAGCTGTTTGAATA
TGCAGGCAAATGGCGTAATATTCGTGTGCAAGGACCGACAACATTTCT
ACCATCCTTGACTGTACAGGTGGCAATGGCAGGTGCCATGTTGATTGG
TCTGCATCATCGCATCTGTTATACGACGAGCGCTTCGGTCTTAACTGA
AGCAGTTAAGCAACCAGATCTTCCTCCAGGTTATGTCCAACTGTGCCA
GCTCGTAATGTCTGGTCAACTTTCCGACCCTGAGAAACTTCTGGAATC
GCTAGAGAATTTCTGGAATGGGGTTCAGGAGTGGGCGGAACGACACGG
ATATATAGTGGATGTGTCAAAACGCATACCATTTTGATGTCTAACCCC
CTTCCTTGCCCACAGCTTCGTCGATGGCGCGAAATTTCGGGTAAATAT
AATGACCCTCTTGATAACCCAAGAGGGCATTTTTTAGGCGCGCCCTAG
GGTGGATCGGCGGACGATTGCAAAAACGAGAGTTTCCACAGCGTAGCT
GCCAGCCAATTGGTACAGGTATGGGCAACGATCGCTAAGAGTAAATTA
TTCGTTGCCACAGCACTATAGGCAAAGAATCCGCCCACAAAGGTAGCC
CACAGGGCATAGGGCCACTGCTGCCGCGATCCAGCGTGCAAAATGCCA
AAGCACGCAGAACTGCCAATAATCCCTGCCCAGTTGAGCCCCAAACTC
GGTAGGAGCACCCCGCGAAAGAGCAGCTCTTCACTAAGGCCGGGCAGA
ATGCCAATCCAAAATAGATCAGGCCACAGCAGTGGTGAAAGCACAAGT
TTCAGGTAGGTATCTGAGGCGTGGCGGTAGGCCGGCCAGAGGCGATAC
AAAATGGCGCCAATGCCGGTAATTCCTAGGCAGAGGGCAATGCCTAAA
ACCACTGCCCAGACATCCCAGCGCAGCGGCAGCAGTCCCCCAGAAAAG
GGGGTAAATAACCACACCCGCGCCAAAATCAGCCACAGGATGGCCGTT
AACGCCATGGCCACTAAGACCTGTGTACGACTCAGAGGCTCATCGGGT
AGGGGGGACTCCTCCATAGGTCTACGCTTTCTGGAACTGACCAAATTG
GAAGTTATAGACCTCCTCCTCTTTTTCAGAGATCAATTTCAAATCTGA
GCAAGGGCGGGCCACACAGAGGAGGACATAGCCTTTTTCCCGCAGTTC
GGGACTCAGCCCCATTGCATCTCCGTGATCCACGGTACCCTCCTGAAT
TTGGGCCGCACAGGTGGTACATACCCCGGCATTGCAGGAACTCGGAAG
ATCAATTCCGGCAGCGGTGGCCGATCGCAGGAGGGGTTTATCGGCACT
GGCTTCAAAAGTGTAGGTTTGTCCTTGGTGCAGAATCTCAACACGAAA
GGTTTGGGTCATTCTGGCAGTGAGCTATGACGCAACATCTTCCCTATT
ATCCCCCTAATCCTCGCGATCGCTGGCTTCCTCGGGGGCAGACTTCAA
CCATGCCGGCAAAGGATCAGGAATCGGCACACGCTGGCGGTGGGGCAG
TTGCAGGCACATGTGTTGCGTCTGGGCAATGGCTACCCGATCCCCCCC
TTCGTTGTAGAGAGTATAGGTCAGTTGAAAACGGCTAGTATCCAGTCT
TTGGGGGTCAATGGTCACCCGCAGGCGATCGCCACAGTAGAGGGGTTT
CAAAAACCGTATCTGCGCCTCCGTAATCGGCACAATGAGGCCACTGTT
GCTGAAAAATTGCCGCAGATCTACCCCCAATTGGGCAAGGGCATCCTC
ATAGGCCTCATGGCAAAACCGCAGCAGATTGGCAAAGTAGACTACCCC
AGCCGCATCGGTATCGGCAAAATGAACTGTGCGCTGATAGTCGCGCAG
GGGTGTTGGATTCATCTATCGTCCTTCCATTGCCATCCCATAGGGTTG
TCCAACACAAGCCATGGGCAAAAACGCGCCACAGCATTTGTTGTTAAT
ATAGGATACAGCTCTTTTGCAACCAATTCCCATCCCTAAACCGATGAG
TAACAAAGGCAGTTCTGATCTGCGACTTCTTTTAAGCACGCTGGTGAT
CAGTGGCTTAGTCGCAGGACTGGCCTATTGGCAACTCAGTCAACACTG
GACCCGCTCCCCCGATCAAAACGCTGGCTCCCCCCTCCACACCCCAAC
CTCAAAGTGGCAAAAAATTGCCCTCGCGATGACCCTGCGGGGCCATGA
AGATGAGGTGAACGCGATCGCCCTGAGTCCCGATGGCAATTTCCTCGT
CAGTGCTGGCGACGATCGCAGGCTGTACTTCTGGAACTTGGCTACGGG
AACTGCCCTAGGACAAGCCAAAGGTCACACCGACTGGATCTATGCCCT
GGTGATGACTCCCGATGGTCAGACGGTGATTAGCGGCAGTAAAGACAA
AACCATCAAACTATGGGGGGTGGGCGATCGCCAACTCCAAGCCACCCT
CAGTGGCCACCAAGATTTTGTGAATGGCTTAGCCCTCAGTCCCGACGG
TCGCACCCTTGCCAGTGCCAGCTATGATCACACCGTCAAACTGTGGAA
TGTTCCCAGCCGTCAGGAAATTACTACGCTCAAAGCAAATGAGGGCAT
CATGCTCAGCGTCGCCATTAGTCGAGATGGGCGTTTTTTAGCCACGGG
TGGCGTGGATAAACTCATCCGCATTTGGGATTTGCCCTCCCGCCGACT
CCTGCGCACCCTGGAAGGACACACCAGTGATGTCAATAGCCTCGCCTT
CACCCCCGACAGCAGCCAACTGGTCAGTGGCAGTGACAAAGATGGTAT
AAAACTTTGGAACCTGACCACAGGAGAACTGCAGCAACAGTTTGGCAC
TGAGGGCGGGCAGGTCTTTAGTGTGGCAGTGAGTCCCGACGGCAGCAC
CCTTGCCAGTGGTCACGGCGATCAAACTGTCAAACTTTGGTCCCTCTC
TGGTCAGTTATTGCGGAACCTCAAGGGACACTCTGGCGCTGTCTACAG
TGTCGTCTTTGGTCAGGATCAACTGATCTCCGCCAGTGAAGACAAAAC
ATCAAAGTGTGGCGTCTTTTTCCCGAAACCCCATAGAGAACTCGCGGG
CCTCACCTACGGCACAAAAAACGGCTAAGATCCCCAAGAATCTTAGCC
ACTGAGAACAACGGCTGGAATTTTTTTAGCCCACACTTCCCTCTAGCT
TCAGGCTCAGCAGGCGATCGGCCTCGACTGCAAATTCCATCGGCAATT
GATTAAAGACATCGCGACAGAAGCCACTAATCATCATTGAGACGGCAT
CTTCAGCGGAAATTCCCCGCTGGGCAAAGTAGAAGAGTTGATCTTCAC
CAATTTTCGATGTCGAAGCCTCATGCTCCACCTGGGCAGTGGGGTTTT
GCACCTGAATATAGGGGAAGGTATTGGCAGCGGCCGTATCCCCAATGA
GCATCGAATCGCATTGGGAGTAGTTGCGTGCCCCTGTGGCCTTGGGGC
CAATTTTCACCAGACCGCGATAGCTATTTTGGGAGTGGCCGGCCGAAA
TGCCCTTAGAGACAATCCTGCTGCGGGTATTTTTCCCAATGTGGATCA
TCTTCGTGCCCGTGTCCGCCTGTTGGTAGTGATTGGTGAGGGCAACGG
AGTAAAATTCTCCCACGGAGTTATCCCCCACCAAGACACAACTGGGGT
ATTTCCAAGTAATGGCAGAACCCGTCTCCACCTGTGTCCAGGAAATCT
TGGAATTGCGGCCGAGGCAGAGTCCCCGCTTCGTCACAAAGTTGTAAA
TGCCCCCTTTGCCATTTTCATCGCCGGCATACCAGTTTTGCACAGTGG
AGTATTTGATTTCGGCATTGTCCAGAGCCACCAGCTCCACCACTGCCG
CATGGAGTTGATTGGTGTCAAACATGGGAGCAGTACAACCCTCAAGAT
AGCTCACGTAGCTCCCGGCATCGGCAATGATCAGGGTGCGCTCAAACT
GACCCGACTCACCGTTATTGATGCGGAAATAGGTGGATAGCTCCATTG
GACAGCGGGTATTCTTGGGAACATAGACGAAGGAGCCATCGGAAAAAA
CTGCGGAGTTCAAGGCAGCATAGAAATTATCGCCAATGGGAACAACAC
TGCCTAAGTATTTCTGCACTAACTCGGGATAGTCCTGGAGCGCTTCAG
AAATGGAGCAAAAAATGATCCCCTGCTTGGCCAACTCCTCGCGGAAGG
TGGTGGCCACTGACACACTATCGAAAATGGCATCTACGGCTACATTGG
TGAGCCGCTTTTGCTCTGAAAGGGGAATCCCTAGTTTTTCAAAGGTTT
CCAGCAGAACGGGATCTACTTCATCCAAGCTTTTTAGCTTTTCCTTCT
GTTTCGGAGCTGAGTAATAGACGATGTCTTGATAATTGATGGGGGGAT
AGCTCACCCGTGGCCATTGGGGCTCGCTCATCTTCAGCCATTGACGAT
AGGCACGCAGGCGAAACTCCAGCATGAACTCTGGCTCGTTCTTCTTGG
CGGAGATGAGGCGAATAATGTCCTCGTTGAGACCTTTGGGAATGGTTT
CCGTCTCAATGGGGGTG
SEQ ID NO: 5
pJB825_PcI_pdc_Km_PEM7_adh
CTAGAGGAGCTTGTTAACAGGCTTACGGCTGTTGGCGGCAGCAACGCG
CTTACCCCATTTGACCAATTCTTCAGTGCAGTCTTCACGACCGATGAA
GCATTCGATCAGGGTTGGGCCGTCGGTGTTTGCCAGAGCAACCTTGAT
GCTTCTGCCAGTTCGCCACCGGTTTTAGCCTTCAGGCCTTTACCAGCA
CCGCTGTCATAACCACCGTTACCGTTGAACACTTCCATCAGACCGGCA
TAATCCCAGTTCTTGATGTTGTTGTACGGACCATCATGGATCATAACT
TCGATGGTGTAACCATAGTTATTGATCAAGAAGATGATAACCGGCAGT
TTCAGGCGAACCATCTGAGCGACTTCCTGAGCCGTCAGCTGGAAGGAA
CCATCACCAACCATGAGGATGTTGCGACGTTCCGGAGCACCGACGGCA
TAACCGAAGGCGGCAGGAACGGACCAACCGATGTGACCCCACTGCATT
TCATATTCAACGCGAGCACCGTTCGGGAGCTTCATGCGCTGAGCATTG
AACCAAGAGTCACCGGTTTCAGCAATAACCGTCGTGTTCGGGGTCAGA
AGAGCTTCGACCTGACGGGCGATTTCTGCGTTGACCAACGGAGCACTC
GGATCAGCCGGAGCGGCTTTCTTCAGTTCACCTGCATTGAGGGATTTG
AAGAAGTCCAAAGCACCGGTTTTCTTGGAAACTTTCTGAGCCAAACGG
GTCAGATAGTCTTTCAGATGAACGCTGGGGAAGCGAACGCCGTTAACG
ACGACAGAACGCGGTTCAGCGAGAACCAGTTTCTTAGGATCAGGAATA
TCCGTCCAACCAGTGGTGGAGTAGTCGTTGAAGACAGGAGCCAGAGCG
ATAACCGCATCGGCTTCTTTCATCGTCTTTTCAACGCCCGGATAGCTG
ACTTCACCCCATGAGGTACCGATGTAATGCGGGTTTTCTTCTGGGAAG
AAGCTTTTTGCAGCAGCCATGGTAGCAACTGCGCCACCGAGAGCATCA
GCAAATTTGACAGCAGCTTCTTCAGCACCAGCTGCGCGCAGCTTGCTG
CCGACGAGGACGGCAACTTTGTCGCGGTTGGCGATGAATTTCAGGGTT
TCTTCAACCGCTGCATTCAAAGAAGCTTCGTCGCTGGCTTCGTCATTG
AACAATGCGCTTGCCGGTCCAGGAGCGGCGCAGGGCATGGAAGCAATG
TTGCAAGCGATTTCGAGATAAACCGGCTTCTTCTCACGAAGAGCAGTT
TTAATCACGTGATCGATTTTAGCCGGAGCTTCTTCTGGGGTGTAAATC
GCTTCAGCTGCGGCCGTGATGTTCTTGGCCATTTCCAACTGATAGTGA
TAGTCGGTTTTGCCAAGAGCGTGATGCAACACGTGACCAGCAGCGTGA
TCATTGTTGTTCGGAGCACCGGAGATCAGGATAACCGGAAGGTTTTCT
GCATAGGCGCCACCGATAGCATCAAATGCGGAAAGCGCACCGACGCTG
TAGGTAACGACGGCTGCTGCTGCGCCTTTGGCACGAGCATAACCTTCT
GCACTGAAACCGCAGTTCAGTTCGTTACAGCAATAAACCTGCTCCATG
TTTTTGTTCAAAAGCAGGTTGTCAAGAAGGACGAGGTTGTAGTCGCCC
GCGACTGCGAAGTGATGCTTGAGACCAATCTGGACAAGCCGCTCCGCT
AAATAGGTACCGACAGTATAACTCATATGTTTTCCTCCAGCAAAAGGA
TCCTGCAACCATTATCACCGCCAGAGGTAAAATAGTCAACACGCACGG
TGTTAGGCCGCATAGGCCAGAGGCGCGCCTGGCCTTCATGGCCTATAA
ACGCAGAAAGGCCCACCCGAAGGTGAGCCAGTGTGACTCTAGTAGAGA
GCGTTCACCGACAAACAACAGATAAAACGAAAGGCCCAGTCTTTCGAC
TGAGCCTTTCGTTTTATTTGATGCCTGGAATACTTCGAAGAGATGCTC
GACGTCCGTATCTCAGGCTAGCTTAGAAGAACTCATCCAGCAGACGGT
AGAAGGCAATGCGCTGAGAATCCGGCGCTGCGATACCGTACAGCACCA
GGAAACGGTCAGCCCATTCACCACCCAGTTCTTCTGCAATATCGCGGG
TAGCGAGGGCGATATCCTGATAGCGATCAGCTACACCCAGACGGCCAC
AGTCAATAAAACCAGAGAAGCGGCCGTTTTCCACCATAATGTTTGGCA
GACAAGCGTCGCCATGCGTTACCACCAGGTCTTCGCCGTCCGGCATGC
GGGCTTTCAGACGTGCAAACAGTTCCGCCGGTGCGAGGCCCTGGTGCT
CTTCATCCAGGTCGTCCTGATCAACCAGACCCGCTTCCATACGAGTGC
GTGCACGTTCAATACGGTGTTTAGCCTGATGGTCAAACGGGCAAGTTG
CCGGGTCCAGGGTGTGCAGACGGCGCATCGCGTCCGCCATGATGGAAA
CTTTTTCTGCCGGAGCGAGGTGGCTGCTCAGCAGATCCTGACCCGGAA
CTTCACCCAGCAGCAGCCAATCGCGACCGGCTTCAGTAACTACGTCCA
GAACTGCCGCGCACGGAACACCAGTCGTCGCGAGCCAGGACAGACGGG
CCGCTTCGTCCTGCAGTTCGTTCAGTGCGCCGGACAGGTCGGTTTTCA
CAAACAGAACCGGACGACCCTGTGCAGACAGACGGAAAACCGCTGCAT
CGCTACAGCCAATAGTCAGCTGAGCCCAGTCGTAACCAAACAGGCGTT
CCACCCAAGCAGCCGGAGAACCAGCATGCAGGCCATCTTGTTCAATCA
TACTCTTCCTTTTTCAATATTATTGAAGCATTTATCAGGGTTATTGTC
TCATGAGCAGATACATATTTGAATGTATTTAGAAAAATAAACAAATAG
GGGTCGGGCCGGCGATAATACGCCGGCCCGTTTTTTTTGGCCATGAAG
GCCAGGCGCGCCTCTGGCCTATGCGGCCTGTTGACAATTAATCATCGG
CATAGTATATCGGCATAGTATAATACGACAAGGTGAGGAACTAACATA
TGTGGGAAACTAAGATTAATATCAACGAAGTCCGTGAGATCCGCGCGA
AAACCACCGTTTACTTTGGTGTTGGTGCTATCAAGAAAATTGATGATA
TCGCTCGCGAGTTCAAAGAAAAAGGTTACGATCGCATCATCGTGATCA
CCGGTAAAGGCGCTTACAAAGCGACCGGTGCATGGGAATACATCGTGC
CTGCTCTGAACAAAAACCAGATTACGTATATCCATTATGATCAGGTGA
CCCCGAACCCGACCGTAGATCAGGTTGACGAAGCGACCAAACAGGCCC
GTGAATTTGGCGCTCGCGCAGTACTGGCTATTGGTGGCGGTTCCCCGA
TCGACGCAGCCAAATCTGTGGCGGTGCTGCTGTCTTATCCGGACAAAA
ACGCTCGTCAGCTGTACCAGCTGGAGTTTACCCCGGTAAAAGCAGCGC
CGATCATCGCCATCAACCTGACCCACGGTACGGGCACCGAAGCGGACC
GCTTCGCGGTTGTATCTATCCCGGAGAAGGCCTACAAACCGGCTATCG
CTTACGATTGCATCTACCCGCTGTACTCTATTGACGACCCGGCTCTGA
TGGTTAAACTGCCGAGCGACCAGACGGCGTACGTTAGCGTGGATGCCC
TGAACCATGTTGTTGAAGCTGCGACCTCCAAAGTTGCATCTCCGTACA
CTATTATCCTGGCAAAAGAAACGGTCCGTCTCATCGCACGCTACCTGC
CTCAGGCCCTGTCTCACCCTGCAGACCTGACCGCGCGTTATTACCTCC
TGTATGCCTCTCTGATCGCCGGTATTGCGTTTGATAACGGCCTGCTGC
ATTTCACCCACGCACTGGAACACCCGCTGTCTGCCGTGAAACCTGAAC
TGGCTCATGGCCTGGGTCTGGGTATGCTCCTGCCTGCGGTAGTTAAAC
AAATTTATCCGGCTACCCCGGAGGTACTGGCGGAAATCCTGGAACCAA
TCGTACCGGATCTGAAAGGCGTTCCGGGCGAGGCTGAGAAAGCGGCGT
CTGGCGTGGCGAAATGGCTGGCTGGTGCAGGCATCACTATGAAACTGA
AAGACGCGGGTTTCCAGGCTGAAGATATCGCGCGTCTGACCGACCTGG
CCTTCACCACTCCATCCCTGGAACTCCTGCTGTCTATGGCACCAGTAA
CTGCTGATCGTGAGCGTGTGAAAGCAATTTACCAGGACGCATTTTGA
SEQ ID NO: 6
pJB825_PEM7_pdcZm_Km_PcI_adhAM
CTAGAGGAGCTTGTTAACAGGCTTACGGCTGTTGGCGGCAGCAACGCG
CTTACCCCATTTGACCAATTCTTCAGTGCAGTCTTCACGACCGATGAA
GCATTCGATCAGGGTTGGGCCGTCGGTGTTTGCCAGAGCAACCTTGAT
AGCTTCTGCCAGTTCGCCACCGGTTTTAGCCTTCAGGCCTTTACCAGC
ACCGCTGTCATAACCACCGTTACCGTTGAACACTTCCATCAGACCGGC
ATAATCCCAGTTCTTGATGTTGTTGTACGGACCATCATGGATCATAAC
TTCGATGGTGTAACCATAGTTATTGATCAAGAAGATGATAACCGGCAG
TTTCAGGCGAACCATCTGAGCGACTTCCTGAGCCGTCAGCTGGAAGGA
ACCATCACCAACCATGAGGATGTTGCGACGTTCCGGAGCACCGACGGC
ATAACCGAAGGCGGCAGGAACGGACCAACCGATGTGACCCCACTGCAT
TTCATATTCAACGCGAGCACCGTTCGGGAGCTTCATGCGCTGAGCATT
GAACCAAGAGTCACCGGTTTCAGCAATAACCGTCGTGTTCGGGGTCAG
AAGAGCTTCGACCTGACGGGCGATTTCTGCGTTGACCAACGGAGCACT
CGGATCAGCCGGAGCGGCTTTCTTCAGTTCACCTGCATTGAGGGATTT
GAAGAAGTCCAAAGCACCGGTTTTCTTGGAAACTTTCTGAGCCAAACG
GGTCAGATAGTCTTTCAGATGAACGCTGGGGAAGCGAACGCCGTTAAC
GACGACAGAACGCGGTTCAGCGAGAACCAGTTTCTTAGGATCAGGAAT
ATCCGTCCAACCAGTGGTGGAGTAGTCGTTGAAGACAGGAGCCAGAGC
GATAACCGCATCGGCTTCTTTCATCGTCTTTTCAACGCCCGGATAGCT
GACTTCACCCCATGAGGTACCGATGTAATGCGGGTTTTCTTCTGGGAA
GAAGCTTTTTGCAGCAGCCATGGTAGCAACTGCGCCACCGAGAGCATC
AGCAAATTTGACAGCAGCTTCTTCAGCACCAGCTGCGCGCAGCTTGCT
GCCGACGAGGACGGCAACTTTGTCGCGGTTGGCGATGAATTTCAGGGT
TTCTTCAACCGCTGCATTCAAAGAAGCTTCGTCGCTGGCTTCGTCATT
GAACAATGCGCTTGCCGGTCCAGGAGCGGCGCAGGGCATGGAAGCAAT
GTTGCAAGCGATTTCGAGATAAACCGGCTTCTTCTCACGAAGAGCAGT
TTTAATCACGTGATCGATTTTAGCCGGAGCTTCTTCTGGGGTGTAAAT
CGCTTCAGCTGCGGCCGTGATGTTCTTGGCCATTTCCAACTGATAGTG
ATAGTCGGTTTTGCCAAGAGCGTGATGCAACACGTGACCAGCAGCGTG
ATCATTGTTGTTCGGAGCACCGGAGATCAGGATAACCGGAAGGTTTTC
TGCATAGGCGCCACCGATAGCATCAAATGCGGAAAGCGCACCGACGCT
GTAGGTAACGACGGCTGCTGCTGCGCCTTTGGCACGAGCATAACCTTC
TGCACTGAAACCGCAGTTCAGTTCGTTACAGCAATAAACCTGCTCCAT
GTTTTTGTTCAAAAGCAGGTTGTCAAGAAGGACGAGGTTGTAGTCGCC
CGCGACTGCGAAGTGATGCTTGAGACCAATCTGGACAAGCCGCTCCGC
TAAATAGGTACCGACAGTATAACTCATATGTTAGTTCCTCACCTTGTC
GTATTATACTATGCCGATATACTATGCCGATGATTAATTGTCAACAGG
CCGCATAGGCCAGAGGCGCGCCTGGCCTTCATGGCCAAAAAAAACGGG
CCGGCGTATTATCGCCGGCCCGACCCCTATTTGTTTATTTTTCTAAAT
ACATTCAAATATGTATCTGCTCATGAGACAATAACCCTGATAAATGCT
TCAATAATATTGAAAAAGGAAGAGTATGATTGAACAAGATGGCCTGCA
TGCTGGTTCTCCGGCTGCTTGGGTGGAACGCCTGTTTGGTTACGACTG
GGCTCAGCTGACTATTGGCTGTAGCGATGCAGCGGTTTTCCGTCTGTC
TGCACAGGGTCGTCCGGTTCTGTTTGTGAAAACCGACCTGTCCGGCGC
ACTGAACGAACTGCAGGACGAAGCGGCCCGTCTGTCCTGGCTCGCGAC
GACTGGTGTTCCGTGCGCGGCAGTTCTGGACGTAGTTACTGAAGCCGG
TCGCGATTGGCTGCTGCTGGGTGAAGTTCCGGGTCAGGATCTGCTGAG
CAGCCACCTCGCTCCGGCAGAAAAAGTTTCCATCATGGCGGACGCGAT
GCGCCGTCTGCACACCCTGGACCCGGCAACTTGCCCGTTTGACCATCA
GGCTAAACACCGTATTGAACGTGCACGCACTCGTATGGAAGCGGGTCT
GGTTGATCAGGACGACCTGGATGAAGAGCACCAGGGCCTCGCACCGGC
GGAACTGTTTGCACGTCTGAAAGCCCGCATGCCGGACGGCGAAGACCT
GGTGGTAACGCATGGCGACGCTTGTCTGCCAAACATTATGGTGGAAAA
CGGCCGCTTCTCTGGTTTTATTGACTGTGGCCGTCTGGGTGTAGCTGA
TCGCTATCAGGATATCGCCCTCGCTACCCGCGATATTGCAGAAGAACT
GGGTGGTGAATGGGCTGACCGTTTCCTGGTGCTGTACGGTATCGCAGC
GCCGGATTCTCAGCGCATTGCCTTCTACCGTCTGCTGGATGAGTTCTT
CTAAGCTAGCCTGAGATACGGACGTCGAGCATCTCTTCGAAGTATTCC
AGGCATCAAATAAAACGAAAGGCTCAGTCGAAAGACTGGGCCTTTCGT
TTTATCTGTTGTTTGTCGGTGAACGCTCTCTACTAGAGTCACACTGGC
TCACCTTCGGGTGGGCCTTTCTGCGTTTATAGGCCATGAAGGCCAGGC
GCGCCTCTGGCCTATGCGGCCTAACACCGTGCGTGTTGACTATTTTAC
CTCTGGCGGTGATAATGGTTGCAGGATCCTTTTGCTGGAGGAAAACAT
ATGTGGGAAACTAAGATTAATATCAACGAAGTCCGTGAGATCCGCGCG
AAAACCACCGTTTACTTTGGTGTTGGTGCTATCAAGAAAATTGATGAT
ATCGCTCGCGAGTTCAAAGAAAAAGGTTACGATCGCATCATCGTGATC
ACCGGTAAAGGCGCTTACAAAGCGACCGGTGCATGGGAATACATCGTG
CCTGCTCTGAACAAAAACCAGATTACGTATATCCATTATGATCAGGTG
ACCCCGAACCCGACCGTAGATCAGGTTGACGAAGCGACCAAACAGGCC
CGTGAATTTGGCGCTCGCGCAGTACTGGCTATTGGTGGCGGTTCCCCG
ATCGACGCAGCCAAATCTGTGGCGGTGCTGCTGTCTTATCCGGACAAA
AACGCTCGTCAGCTGTACCAGCTGGAGTTTACCCCGGTAAAAGCAGCG
CCGATCATCGCCATCAACCTGACCCACGGTACGGGCACCGAAGCGGAC
CGCTTCGCGGTTGTATCTATCCCGGAGAAGGCCTACAAACCGGCTATC
GCTTACGATTGCATCTACCCGCTGTACTCTATTGACGACCCGGCTCTG
ATGGTTAAACTGCCGAGCGACCAGACGGCGTACGTTAGCGTGGATGCC
CTGAACCATGTTGTTGAAGCTGCGACCTCCAAAGTTGCATCTCCGTAC
ACTATTATCCTGGCAAAAGAAACGGTCCGTCTCATCGCACGCTACCTG
CCTCAGGCCCTGTCTCACCCTGCAGACCTGACCGCGCGTTATTACCTC
CTGTATGCCTCTCTGATCGCCGGTATTGCGTTTGATAACGGCCTGCTG
CATTTCACCCACGCACTGGAACACCCGCTGTCTGCCGTGAAACCTGAA
CTGGCTCATGGCCTGGGTCTGGGTATGCTCCTGCCTGCGGTAGTTAAA
CAAATTTATCCGGCTACCCCGGAGGTACTGGCGGAAATCCTGGAACCA
ATCGTACCGGATCTGAAAGGCGTTCCGGGCGAGGCTGAGAAAGCGGCG
TCTGGCGTGGCGAAATGGCTGGCTGGTGCAGGCATCACTATGAAACTG
AAAGACGCGGGTTTCCAGGCTGAAGATATCGCGCGTCTGACCGACCTG
GCCTTCACCACTCCATCCCTGGAACTCCTGCTGTCTATGGCACCAGTA
ACTGCTGATCGTGAGCGTGTGAAAGCAATTTACCAGGACGCATTTTGA
SEQ ID NO: 7
pJB825_PcI_pdcZm_Km_PtRNAglu_adhAM
CTAGAGGAGCTTGTTAACAGGCTTACGGCTGTTGGCGGCAGCAACGCG
CTTACCCCATTTGACCAATTCTTCAGTGCAGTCTTCACGACCGATGAA
GCATTCGATCAGGGTTGGGCCGTCGGTGTTTGCCAGAGCAACCTTGAT
AGCTTCTGCCAGTTCGCCACCGGTTTTAGCCTTCAGGCCTTTACCAGC
ACCGCTGTCATAACCACCGTTACCGTTGAACACTTCCATCAGACCGGC
ATAATCCCAGTTCTTGATGTTGTTGTACGGACCATCATGGATCATAAC
TTCGATGGTGTAACCATAGTTATTGATCAAGAAGATGATAACCGGCAG
TTTCAGGCGAACCATCTGAGCGACTTCCTGAGCCGTCAGCTGGAAGGA
ACCATCACCAACCATGAGGATGTTGCGACGTTCCGGAGCACCGACGGC
ATAACCGAAGGCGGCAGGAACGGACCAACCGATGTGACCCCACTGCAT
TTCATATTCAACGCGAGCACCGTTCGGGAGCTTCATGCGCTGAGCATT
GAACCAAGAGTCACCGGTTTCAGCAATAACCGTCGTGTTCGGGGTCAG
AAGAGCTTCGACCTGACGGGCGATTTCTGCGTTGACCAACGGAGCACT
CGGATCAGCCGGAGCGGCTTTCTTCAGTTCACCTGCATTGAGGGATTT
GAAGAAGTCCAAAGCACCGGTTTTCTTGGAAACTTTCTGAGCCAAACG
GGTCAGATAGTCTTTCAGATGAACGCTGGGGAAGCGAACGCCGTTAAC
GACGACAGAACGCGGTTCAGCGAGAACCAGTTTCTTAGGATCAGGAAT
ATCCGTCCAACCAGTGGTGGAGTAGTCGTTGAAGACAGGAGCCAGAGC
GATAACCGCATCGGCTTCTTTCATCGTCTTTTCAACGCCCGGATAGCT
GACTTCACCCCATGAGGTACCGATGTAATGCGGGTTTTCTTCTGGGAA
GAAGCTTTTTGCAGCAGCCATGGTAGCAACTGCGCCACCGAGAGCATC
AGCAAATTTGACAGCAGCTTCTTCAGCACCAGCTGCGCGCAGCTTGCT
GCCGACGAGGACGGCAACTTTGTCGCGGTTGGCGATGAATTTCAGGGT
TTCTTCAACCGCTGCATTCAAAGAAGCTTCGTCGCTGGCTTCGTCATT
GAACAATGCGCTTGCCGGTCCAGGAGCGGCGCAGGGCATGGAAGCAAT
GTTGCAAGCGATTTCGAGATAAACCGGCTTCTTCTCACGAAGAGCAGT
TTTAATCACGTGATCGATTTTAGCCGGAGCTTCTTCTGGGGTGTAAAT
CGCTTCAGCTGCGGCCGTGATGTTCTTGGCCATTTCCAACTGATAGTG
ATAGTCGGTTTTGCCAAGAGCGTGATGCAACACGTGACCAGCAGCGTG
ATCATTGTTGTTCGGAGCACCGGAGATCAGGATAACCGGAAGGTTTTC
TGCATAGGCGCCACCGATAGCATCAAATGCGGAAAGCGCACCGACGCT
GTAGGTAACGACGGCTGCTGCTGCGCCTTTGGCACGAGCATAACCTTC
TGCACTGAAACCGCAGTTCAGTTCGTTACAGCAATAAACCTGCTCCAT
GTTTTTGTTCAAAAGCAGGTTGTCAAGAAGGACGAGGTTGTAGTCGCC
CGCGACTGCGAAGTGATGCTTGAGACCAATCTGGACAAGCCGCTCCGC
TAAATAGGTACCGACAGTATAACTCATATGTTTTCCTCCAGCAAAAGG
ATCCTGCAACCATTATCACCGCCAGAGGTAAAATAGTCAACACGCACG
GTGTTAGGCCGCATAGGCCAGAGGCGCGCCTGGCCTTCATGGCCTATA
AACGCAGAAAGGCCCACCCGAAGGTGAGCCAGTGTGACTCTAGTAGAG
AGCGTTCACCGACAAACAACAGATAAAACGAAAGGCCCAGTCTTTCGA
CTGAGCCTTTCGTTTTATTTGATGCCTGGAATACTTCGAAGAGATGCT
CGACGTCCGTATCTCAGGCTAGCTTAGAAGAACTCATCCAGCAGACGG
TAGAAGGCAATGCGCTGAGAATCCGGCGCTGCGATACCGTACAGCACC
AGGAAACGGTCAGCCCATTCACCACCCAGTTCTTCTGCAATATCGCGG
GTAGCGAGGGCGATATCCTGATAGCGATCAGCTACACCCAGACGGCCA
CAGTCAATAAAACCAGAGAAGCGGCCGTTTTCCACCATAATGTTTGGC
AGACAAGCGTCGCCATGCGTTACCACCAGGTCTTCGCCGTCCGGCATG
CGGGCTTTCAGACGTGCAAACAGTTCCGCCGGTGCGAGGCCCTGGTGC
TCTTCATCCAGGTCGTCCTGATCAACCAGACCCGCTTCCATACGAGTG
CGTGCACGTTCAATACGGTGTTTAGCCTGATGGTCAAACGGGCAAGTT
GCCGGGTCCAGGGTGTGCAGACGGCGCATCGCGTCCGCCATGATGGAA
ACTTTTTCTGCCGGAGCGAGGTGGCTGCTCAGCAGATCCTGACCCGGA
ACTTCACCCAGCAGCAGCCAATCGCGACCGGCTTCAGTAACTACGTCC
AGAACTGCCGCGCACGGAACACCAGTCGTCGCGAGCCAGGACAGACGG
GCCGCTTCGTCCTGCAGTTCGTTCAGTGCGCCGGACAGGTCGGTTTTC
ACAAACAGAACCGGACGACCCTGTGCAGACAGACGGAAAACCGCTGCA
TCGCTACAGCCAATAGTCAGCTGAGCCCAGTCGTAACCAAACAGGCGT
TCCACCCAAGCAGCCGGAGAACCAGCATGCAGGCCATCTTGTTCAATC
ATACTCTTCCTTTTTCAATATTATTGAAGCATTTATCAGGGTTATTGT
CTCATGAGCAGATACATATTTGAATGTATTTAGAAAAATAAACAAATA
GGGGTCGGGCCGGCGATAATACGCCGGCCCGTTTTTTTTGGCCATGAA
GGCCAGGCGCGCCTCTGGCCTATGCGGCCTCGCCCTCATTTTCTCCCT
AGGAGGGGCTTCGATGCAAAAATTGCCCGAGGTGTTGACAAACGCTCA
GGGTATTCGCTACATTAACTAATGCTGAGTCTTGATCTAAAGATCTTT
CTAGATTCTCGAGGCATATGTGGGAAACTAAGATTAATATCAACGAAG
TCCGTGAGATCCGCGCGAAAACCACCGTTTACTTTGGTGTTGGTGCTA
TCAAGAAAATTGATGATATCGCTCGCGAGTTCAAAGAAAAAGGTTACG
ATCGCATCATCGTGATCACCGGTAAAGGCGCTTACAAAGCGACCGGTG
CATGGGAATACATCGTGCCTGCTCTGAACAAAAACCAGATTACGTATA
TCCATTATGATCAGGTGACCCCGAACCCGACCGTAGATCAGGTTGACG
AAGCGACCAAACAGGCCCGTGAATTTGGCGCTCGCGCAGTACTGGCTA
TTGGTGGCGGTTCCCCGATCGACGCAGCCAAATCTGTGGCGGTGCTGC
TGTCTTATCCGGACAAAAACGCTCGTCAGCTGTACCAGCTGGAGTTTA
CCCCGGTAAAAGCAGCGCCGATCATCGCCATCAACCTGACCCACGGTA
CGGGCACCGAAGCGGACCGCTTCGCGGTTGTATCTATCCCGGAGAAGG
CCTACAAACCGGCTATCGCTTACGATTGCATCTACCCGCTGTACTCTA
TTGACGACCCGGCTCTGATGGTTAAACTGCCGAGCGACCAGACGGCGT
ACGTTAGCGTGGATGCCCTGAACCATGTTGTTGAAGCTGCGACCTCCA
AAGTTGCATCTCCGTACACTATTATCCTGGCAAAAGAAACGGTCCGTC
TCATCGCACGCTACCTGCCTCAGGCCCTGTCTCACCCTGCAGACCTGA
CCGCGCGTTATTACCTCCTGTATGCCTCTCTGATCGCCGGTATTGCGT
TTGATAACGGCCTGCTGCATTTCACCCACGCACTGGAACACCCGCTGT
CTGCCGTGAAACCTGAACTGGCTCATGGCCTGGGTCTGGGTATGCTCC
TGCCTGCGGTAGTTAAACAAATTTATCCGGCTACCCCGGAGGTACTGG
CGGAAATCCTGGAACCAATCGTACCGGATCTGAAAGGCGTTCCGGGCG
AGGCTGAGAAAGCGGCGTCTGGCGTGGCGAAATGGCTGGCTGGTGCAG
GCATCACTATGAAACTGAAAGACGCGGGTTTCCAGGCTGAAGATATCG
CGCGTCTGACCGACCTGGCCTTCACCACTCCATCCCTGGAACTCCTGC
TGTCTATGGCACCAGTAACTGCTGATCGTGAGCGTGTGAAAGCAATTT
ACCAGGACGCATTTTGA
SEQ ID NO: 8
pJB825_PtRNAglu_pdcZm_Km_PcI_adhAM
CTAGAGGAGCTTGTTAACAGGCTTACGGCTGTTGGCGGCAGCAACGCG
CTTACCCCATTTGACCAATTCTTCAGTGCAGTCTTCACGACCGATGAA
GCATTCGATCAGGGTTGGGCCGTCGGTGTTTGCCAGAGCAACCTTGAT
AGCTTCTGCCAGTTCGCCACCGGTTTTAGCCTTCAGGCCTTTACCAGC
ACCGCTGTCATAACCACCGTTACCGTTGAACACTTCCATCAGACCGGC
ATAATCCCAGTTCTTGATGTTGTTGTACGGACCATCATGGATCATAAC
TTCGATGGTGTAACCATAGTTATTGATCAAGAAGATGATAACCGGCAG
TTTCAGGCGAACCATCTGAGCGACTTCCTGAGCCGTCAGCTGGAAGGA
ACCATCACCAACCATGAGGATGTTGCGACGTTCCGGAGCACCGACGGC
ATAACCGAAGGCGGCAGGAACGGACCAACCGATGTGACCCCACTGCAT
TTCATATTCAACGCGAGCACCGTTCGGGAGCTTCATGCGCTGAGCATT
GAACCAAGAGTCACCGGTTTCAGCAATAACCGTCGTGTTCGGGGTCAG
AAGAGCTTCGACCTGACGGGCGATTTCTGCGTTGACCAACGGAGCACT
CGGATCAGCCGGAGCGGCTTTCTTCAGTTCACCTGCATTGAGGGATTT
GAAGAAGTCCAAAGCACCGGTTTTCTTGGAAACTTTCTGAGCCAAACG
GGTCAGATAGTCTTTCAGATGAACGCTGGGGAAGCGAACGCCGTTAAC
GACGACAGAACGCGGTTCAGCGAGAACCAGTTTCTTAGGATCAGGAAT
ATCCGTCCAACCAGTGGTGGAGTAGTCGTTGAAGACAGGAGCCAGAGC
GATAACCGCATCGGCTTCTTTCATCGTCTTTTCAACGCCCGGATAGCT
GACTTCACCCCATGAGGTACCGATGTAATGCGGGTTTTCTTCTGGGAA
GAAGCTTTTTGCAGCAGCCATGGTAGCAACTGCGCCACCGAGAGCATC
AGCAAATTTGACAGCAGCTTCTTCAGCACCAGCTGCGCGCAGCTTGCT
GCCGACGAGGACGGCAACTTTGTCGCGGTTGGCGATGAATTTCAGGGT
TTCTTCAACCGCTGCATTCAAAGAAGCTTCGTCGCTGGCTTCGTCATT
GAACAATGCGCTTGCCGGTCCAGGAGCGGCGCAGGGCATGGAAGCAAT
GTTGCAAGCGATTTCGAGATAAACCGGCTTCTTCTCACGAAGAGCAGT
TTTAATCACGTGATCGATTTTAGCCGGAGCTTCTTCTGGGGTGTAAAT
CGCTTCAGCTGCGGCCGTGATGTTCTTGGCCATTTCCAACTGATAGTG
ATAGTCGGTTTTGCCAAGAGCGTGATGCAACACGTGACCAGCAGCGTG
ATCATTGTTGTTCGGAGCACCGGAGATCAGGATAACCGGAAGGTTTTC
TGCATAGGCGCCACCGATAGCATCAAATGCGGAAAGCGCACCGACGCT
GTAGGTAACGACGGCTGCTGCTGCGCCTTTGGCACGAGCATAACCTTC
TGCACTGAAACCGCAGTTCAGTTCGTTACAGCAATAAACCTGCTCCAT
GTTTTTGTTCAAAAGCAGGTTGTCAAGAAGGACGAGGTTGTAGTCGCC
CGCGACTGCGAAGTGATGCTTGAGACCAATCTGGACAAGCCGCTCCGC
TAAATAGGTACCGACAGTATAACTCATATGCCTCGAGAATCTAGAAAG
ATCTTTAGATCAAGACTCAGCATTAGTTAATGTAGCGAATACCCTGAG
CGTTTGTCAACACCTCGGGCAATTTTTGCATCGAAGCCCCTCCTAGGG
AGAAAATGAGGGCGAGGCCGCATAGGCCAGAGGCGCGCCTGGCCTTCA
TGGCCAAAAAAAACGGGCCGGCGTATTATCGCCGGCCCGACCCCTATT
TGTTTATTTTTCTAAATACATTCAAATATGTATCTGCTCATGAGACAA
TAACCCTGATAAATGCTTCAATAATATTGAAAAAGGAAGAGTATGATT
GAACAAGATGGCCTGCATGCTGGTTCTCCGGCTGCTTGGGTGGAACGC
CTGTTTGGTTACGACTGGGCTCAGCTGACTATTGGCTGTAGCGATGCA
GCGGTTTTCCGTCTGTCTGCACAGGGTCGTCCGGTTCTGTTTGTGAAA
ACCGACCTGTCCGGCGCACTGAACGAACTGCAGGACGAAGCGGCCCGT
CTGTCCTGGCTCGCGACGACTGGTGTTCCGTGCGCGGCAGTTCTGGAC
GTAGTTACTGAAGCCGGTCGCGATTGGCTGCTGCTGGGTGAAGTTCCG
GGTCAGGATCTGCTGAGCAGCCACCTCGCTCCGGCAGAAAAAGTTTCC
ATCATGGCGGACGCGATGCGCCGTCTGCACACCCTGGACCCGGCAACT
TGCCCGTTTGACCATCAGGCTAAACACCGTATTGAACGTGCACGCACT
CGTATGGAAGCGGGTCTGGTTGATCAGGACGACCTGGATGAAGAGCAC
CAGGGCCTCGCACCGGCGGAACTGTTTGCACGTCTGAAAGCCCGCATG
CCGGACGGCGAAGACCTGGTGGTAACGCATGGCGACGCTTGTCTGCCA
AACATTATGGTGGAAAACGGCCGCTTCTCTGGTTTTATTGACTGTGGC
CGTCTGGGTGTAGCTGATCGCTATCAGGATATCGCCCTCGCTACCCGC
GATATTGCAGAAGAACTGGGTGGTGAATGGGCTGACCGTTTCCTGGTG
CTGTACGGTATCGCAGCGCCGGATTCTCAGCGCATTGCCTTCTACCGT
CTGCTGGATGAGTTCTTCTAAGCTAGCCTGAGATACGGACGTCGAGCA
TCTCTTCGAAGTATTCCAGGCATCAAATAAAACGAAAGGCTCAGTCGA
AAGACTGGGCCTTTCGTTTTATCTGTTGTTTGTCGGTGAACGCTCTCT
ACTAGAGTCACACTGGCTCACCTTCGGGTGGGCCTTTCTGCGTTTATA
GGCCATGAAGGCCAGGCGCGCCTCTGGCCTATGCGGCCTAACACCGTG
CGTGTTGACTATTTTACCTCTGGCGGTGATAATGGTTGCAGGATCCTT
TTGCTGGAGGAAAACATATGTGGGAAACTAAGATTAATATCAACGAAG
TCCGTGAGATCCGCGCGAAAACCACCGTTTACTTTGGTGTTGGTGCTA
TCAAGAAAATTGATGATATCGCTCGCGAGTTCAAAGAAAAAGGTTACG
ATCGCATCATCGTGATCACCGGTAAAGGCGCTTACAAAGCGACCGGTG
CATGGGAATACATCGTGCCTGCTCTGAACAAAAACCAGATTACGTATA
TCCATTATGATCAGGTGACCCCGAACCCGACCGTAGATCAGGTTGACG
AAGCGACCAAACAGGCCCGTGAATTTGGCGCTCGCGCAGTACTGGCTA
TTGGTGGCGGTTCCCCGATCGACGCAGCCAAATCTGTGGCGGTGCTGC
TGTCTTATCCGGACAAAAACGCTCGTCAGCTGTACCAGCTGGAGTTTA
CCCCGGTAAAAGCAGCGCCGATCATCGCCATCAACCTGACCCACGGTA
CGGGCACCGAAGCGGACCGCTTCGCGGTTGTATCTATCCCGGAGAAGG
CCTACAAACCGGCTATCGCTTACGATTGCATCTACCCGCTGTACTCTA
TTGACGACCCGGCTCTGATGGTTAAACTGCCGAGCGACCAGACGGCGT
ACGTTAGCGTGGATGCCCTGAACCATGTTGTTGAAGCTGCGACCTCCA
AAGTTGCATCTCCGTACACTATTATCCTGGCAAAAGAAACGGTCCGTC
TCATCGCACGCTACCTGCCTCAGGCCCTGTCTCACCCTGCAGACCTGA
CCGCGCGTTATTACCTCCTGTATGCCTCTCTGATCGCCGGTATTGCGT
TTGATAACGGCCTGCTGCATTTCACCCACGCACTGGAACACCCGCTGT
CTGCCGTGAAACCTGAACTGGCTCATGGCCTGGGTCTGGGTATGCTCC
TGCCTGCGGTAGTTAAACAAATTTATCCGGCTACCCCGGAGGTACTGG
CGGAAATCCTGGAACCAATCGTACCGGATCTGAAAGGCGTTCCGGGCG
AGGCTGAGAAAGCGGCGTCTGGCGTGGCGAAATGGCTGGCTGGTGCAG
GCATCACTATGAAACTGAAAGACGCGGGTTTCCAGGCTGAAGATATCG
CGCGTCTGACCGACCTGGCCTTCACCACTCCATCCCTGGAACTCCTGC
TGTCTATGGCACCAGTAACTGCTGATCGTGAGCGTGTGAAAGCAATTT
ACCAGGACGCATTTTGA
SEQ ID NO: 9
pJB826_PaphII_pdcZp_PcI_adhAm
GCGGCCGCGGGGGGGGGGGGGAAAGCCACGTTGTGTCTCAAAATCTCT
GATGTTACATTGCACAAGATAAAAATATATCATCATGAACAATAAAAC
TGTCTGCTTACATAAACAGTAATACAAGGGGTCATATGTATACCGTTG
GTATGTACTTGGCAGAACGCCTAGCCCAGATCGGCCTGAAACACCACT
TTGCCGTGGCCGGTGACTACAACCTGGTGTTGCTTGATCAGCTCCTGC
TGAACAAAGACATGGAGCAGGTCTACTGCTGTAACGAACTTAACTGCG
GCTTTAGCGCCGAAGGTTACGCTCGTGCACGTGGTGCCGCCGCTGCCA
TCGTCACGTTCAGCGTAGGTGCTATCTCTGCAATGAACGCCATCGGTG
GCGCCTATGCAGAAAACCTGCCGGTCATCCTGATCTCTGGCTCACCGA
ACACCAATGACTACGGCACAGGCCACATCCTGCACCACACCATTGGTA
CTACTGACTATAACTATCAGCTGGAAATGGTAAAACACGTTACCTGCG
CACGTGAAAGCATCGTTTCTGCCGAAGAAGCACCGGCAAAAATCGACC
ACGTCATCCGTACGGCTCTACGTGAACGCAAACCGGCTTATCTGGAAA
TCGCATGCAACGTCGCTGGCGCTGAATGTGTTCGTCCGGGCCCGATCA
ATAGCCTGCTGCGTGAACTCGAAGTTGACCAGACCAGTGTCACTGCCG
CTGTAGATGCCGCCGTAGAATGGCTGCAGGACCGCCAGAACGTCGTCA
TGCTGGTCGGTAGCAAACTGCGTGCCGCTGCCGCTGAAAAACAGGCTG
TTGCCCTAGCGGACCGCCTGGGCTGCGCTGTCACGATCATGGCTGCCG
AAAAAGGCTTCTTCCCGGAAGATCATCCGAACTTCCGCGGCCTGTACT
GGGGTGAAGTCAGCTCCGAAGGTGCACAGGAACTGGTTGAAAACGCCG
ATGCCATCCTGTGTCTGGCACCGGTATTCAACGACTATGCTACCGTTG
GCTGGAACTCCTGGCCGAAAGGCGACAATGTCATGGTCATGGACACCG
ACCGCGTCACTTTCGCAGGACAGTCCTTCGAAGGTCTGTCATTGAGCA
CCTTCGCCGCAGCACTGGCTGAGAAAGCACCTTCTCGCCCGGCAACGA
CTCAAGGCACTCAAGCACCGGTACTGGGTATTGAGGCCGCAGAGCCCA
ATGCACCGCTGACCAATGACGAAATGACGCGTCAGATCCAGTCGCTGA
TCACTTCCGACACTACTCTGACAGCAGAAACAGGTGACTCTTGGTTCA
ACGCTTCTCGCATGCCGATTCCTGGCGGTGCTCGTGTCGAACTGGAAA
TGCAATGGGGTCATATCGGTTGGTCCGTACCTTCTGCATTCGGTAACG
CCGTTGGTTCTCCGGAGCGTCGCCACATCATGATGGTCGGTGATGGCT
CTTTCCAGCTGACTGCTCAAGAAGTTGCTCAGATGATCCGCTATGAAA
TCCCGGTCATCATCTTCCTGATCAACAACCGCGGTTACGTCATCGAAA
TCGCTATCCATGACGGCCCTTACAACTACATCAAAAACTGGAACTACG
CTGGCCTGATCGACGTCTTCAATGACGAAGATGGTCATGGCCTGGGTC
TGAAAGCTTCTACTGGTGCAGAACTAGAAGGCGCTATCAAGAAAGCAC
TCGACAATCGTCGCGGTCCGACGCTGATCGAATGTAACATCGCTCAGG
ACGACTGCACTGAAACCCTGATTGCTTGGGGTAAACGTGTAGCAGCTA
CCAACTCTCGCAAACCACAAGCGTAATTAACTCGAGTAACACCGTGCG
TGTTGACTATTTTACCTCTGGCGGTGATAATGGTTGCAGGATCCTTTT
GCTGGAGGAAAACCATATGTGGGAAACTAAGATTAATATCAACGAAGT
CCGTGAGATCCGCGCGAAAACCACCGTTTACTTTGGTGTTGGTGCTAT
CAAGAAAATTGATGATATCGCTCGCGAGTTCAAAGAAAAAGGTTACGA
TCGCATCATCGTGATCACCGGTAAAGGCGCTTACAAAGCGACCGGTGC
ATGGGAATACATCGTGCCTGCTCTGAACAAAAACCAGATTACGTATAT
CCATTATGATCAGGTGACCCCGAACCCGACCGTAGATCAGGTTGACGA
AGCGACCAAACAGGCCCGTGAATTTGGCGCTCGCGCAGTACTGGCTAT
TGGTGGCGGTTCCCCGATCGACGCAGCCAAATCTGTGGCGGTGCTGCT
GTCTTATCCGGACAAAAACGCTCGTCAGCTGTACCAGCTGGAGTTTAC
CCCGGTAAAAGCAGCGCCGATCATCGCCATCAACCTGACCCACGGTAC
GGGCACCGAAGCGGACCGCTTCGCGGTTGTATCTATCCCGGAGAAGGC
CTACAAACCGGCTATCGCTTACGATTGCATCTACCCGCTGTACTCTAT
TGACGACCCGGCTCTGATGGTTAAACTGCCGAGCGACCAGACGGCGTA
CGTTAGCGTGGATGCCCTGAACCATGTTGTTGAAGCTGCGACCTCCAA
AGTTGCATCTCCGTACACTATTATCCTGGCAAAAGAAACGGTCCGTCT
CATCGCACGCTACCTGCCTCAGGCCCTGTCTCACCCTGCAGACCTGAC
CGCGCGTTATTACCTCCTGTATGCCTCTCTGATCGCCGGTATTGCGTT
TGATAACGGCCTGCTGCATTTCACCCACGCACTGGAACACCCGCTGTC
TGCCGTGAAACCTGAACTGGCTCATGGCCTGGGTCTGGGTATGCTCCT
GCCTGCGGTAGTTAAACAAATTTATCCGGCTACCCCGGAGGTACTGGC
GGAAATCCTGGAACCAATCGTACCGGATCTGAAAGGCGTTCCGGGCGA
GGCTGAGAAAGCGGCGTCTGGCGTGGCGAAATGGCTGGCTGGTGCAGG
CATCACTATGAAACTGAAAGACGCGGGTTTCCAGGCTGAAGATATCGC
GCGTCTGACCGACCTGGCCTTCACCACTCCATCCCTGGAACTCCTGCT
GTCTATGGCACCAGTAACTGCTGATCGTGAGCGTGTGAAAGCAATTTA
CCAGGACGCATTTTGAGCGGCCGC
SEQ ID NO: 10
pJB826_PcpcB_pdcZp_PcI_adhAm
GCGGCCGCTTCGTTATAAAATAAACTTAACAAATCTATACCCACCTGT
AGAGAAGAGTCCCTGAATATCAAAATGGTGGGATAAAAAGCTCAAAAA
GGAAAGTAGGCTGTGGTTCCCTAGGCAACAGTCTTCCCTACCCCACTG
GAAACTAAAAAAACGAGAAAAGTTCGCACCGAACATCAATTGCATAAT
TTTAGCCCTAAAACATAAGCTGAACGAAACTGGTTGTCTTCCCTTCCC
AATCCAGGACAATCTGAGAATCCCCTGCAACATTACTTAACAAAAAAG
CAGGAATAAAATTAACAAGATGTAACAGACATAAGTCCCATCACCGTT
GTATAAAGTTAACTGTGGGATTGCAAAAGCATTCAAGCCTAGGCGCTG
AGCTGTTTGAGCATCCCGGTGGCCCTTGTCGCTGCCTCCGTGTTTCTC
CCTGGATTTATTTAGGTAATATCTCTCATAAATCCCCGGGTAGTTAAC
GAAAGTTAATGGAGATCAGTAACAATAACTCTAGGGTCATTACTTTGG
ACTCCCTCAGTTTATCCGGGGGAATTGTGTTTAAGAAAATCCCAACTC
ATAAAGTCAAGTAGGAGATTAATCATATGTATACCGTTGGTATGTACT
TGGCAGAACGCCTAGCCCAGATCGGCCTGAAACACCACTTTGCCGTGG
CCGGTGACTACAACCTGGTGTTGCTTGATCAGCTCCTGCTGAACAAAG
ACATGGAGCAGGTCTACTGCTGTAACGAACTTAACTGCGGCTTTAGCG
CCGAAGGTTACGCTCGTGCACGTGGTGCCGCCGCTGCCATCGTCACGT
TCAGCGTAGGTGCTATCTCTGCAATGAACGCCATCGGTGGCGCCTATG
CAGAAAACCTGCCGGTCATCCTGATCTCTGGCTCACCGAACACCAATG
ACTACGGCACAGGCCACATCCTGCACCACACCATTGGTACTACTGACT
ATAACTATCAGCTGGAAATGGTAAAACACGTTACCTGCGCACGTGAAA
GCATCGTTTCTGCCGAAGAAGCACCGGCAAAAATCGACCACGTCATCC
GTACGGCTCTACGTGAACGCAAACCGGCTTATCTGGAAATCGCATGCA
ACGTCGCTGGCGCTGAATGTGTTCGTCCGGGCCCGATCAATAGCCTGC
TGCGTGAACTCGAAGTTGACCAGACCAGTGTCACTGCCGCTGTAGATG
CCGCCGTAGAATGGCTGCAGGACCGCCAGAACGTCGTCATGCTGGTCG
GTAGCAAACTGCGTGCCGCTGCCGCTGAAAAACAGGCTGTTGCCCTAG
CGGACCGCCTGGGCTGCGCTGTCACGATCATGGCTGCCGAAAAAGGCT
TCTTCCCGGAAGATCATCCGAACTTCCGCGGCCTGTACTGGGGTGAAG
TCAGCTCCGAAGGTGCACAGGAACTGGTTGAAAACGCCGATGCCATCC
TGTGTCTGGCACCGGTATTCAACGACTATGCTACCGTTGGCTGGAACT
CCTGGCCGAAAGGCGACAATGTCATGGTCATGGACACCGACCGCGTCA
CTTTCGCAGGACAGTCCTTCGAAGGTCTGTCATTGAGCACCTTCGCCG
CAGCACTGGCTGAGAAAGCACCTTCTCGCCCGGCAACGACTCAAGGCA
CTCAAGCACCGGTACTGGGTATTGAGGCCGCAGAGCCCAATGCACCGC
TGACCAATGACGAAATGACGCGTCAGATCCAGTCGCTGATCACTTCCG
ACACTACTCTGACAGCAGAAACAGGTGACTCTTGGTTCAACGCTTCTC
GCATGCCGATTCCTGGCGGTGCTCGTGTCGAACTGGAAATGCAATGGG
GTCATATCGGTTGGTCCGTACCTTCTGCATTCGGTAACGCCGTTGGTT
CTCCGGAGCGTCGCCACATCATGATGGTCGGTGATGGCTCTTTCCAGC
TGACTGCTCAAGAAGTTGCTCAGATGATCCGCTATGAAATCCCGGTCA
TCATCTTCCTGATCAACAACCGCGGTTACGTCATCGAAATCGCTATCC
ATGACGGCCCTTACAACTACATCAAAAACTGGAACTACGCTGGCCTGA
TCGACGTCTTCAATGACGAAGATGGTCATGGCCTGGGTCTGAAAGCTT
CTACTGGTGCAGAACTAGAAGGCGCTATCAAGAAAGCACTCGACAATC
GTCGCGGTCCGACGCTGATCGAATGTAACATCGCTCAGGACGACTGCA
CTGAAACCCTGATTGCTTGGGGTAAACGTGTAGCAGCTACCAACTCTC
GCAAACCACAAGCGTAATTAACTCGAGTAACACCGTGCGTGTTGACTA
TTTTACCTCTGGCGGTGATAATGGTTGCAGGATCCTTTTGCTGGAGGA
AAACCATATGTGGGAAACTAAGATTAATATCAACGAAGTCCGTGAGAT
CCGCGCGAAAACCACCGTTTACTTTGGTGTTGGTGCTATCAAGAAAAT
TGATGATATCGCTCGCGAGTTCAAAGAAAAAGGTTACGATCGCATCAT
CGTGATCACCGGTAAAGGCGCTTACAAAGCGACCGGTGCATGGGAATA
CATCGTGCCTGCTCTGAACAAAAACCAGATTACGTATATCCATTATGA
TCAGGTGACCCCGAACCCGACCGTAGATCAGGTTGACGAAGCGACCAA
ACAGGCCCGTGAATTTGGCGCTCGCGCAGTACTGGCTATTGGTGGCGG
TTCCCCGATCGACGCAGCCAAATCTGTGGCGGTGCTGCTGTCTTATCC
GGACAAAAACGCTCGTCAGCTGTACCAGCTGGAGTTTACCCCGGTAAA
AGCAGCGCCGATCATCGCCATCAACCTGACCCACGGTACGGGCACCGA
AGCGGACCGCTTCGCGGTTGTATCTATCCCGGAGAAGGCCTACAAACC
GGCTATCGCTTACGATTGCATCTACCCGCTGTACTCTATTGACGACCC
GGCTCTGATGGTTAAACTGCCGAGCGACCAGACGGCGTACGTTAGCGT
GGATGCCCTGAACCATGTTGTTGAAGCTGCGACCTCCAAAGTTGCATC
TCCGTACACTATTATCCTGGCAAAAGAAACGGTCCGTCTCATCGCACG
CTACCTGCCTCAGGCCCTGTCTCACCCTGCAGACCTGACCGCGCGTTA
TTACCTCCTGTATGCCTCTCTGATCGCCGGTATTGCGTTTGATAACGG
CCTGCTGCATTTCACCCACGCACTGGAACACCCGCTGTCTGCCGTGAA
ACCTGAACTGGCTCATGGCCTGGGTCTGGGTATGCTCCTGCCTGCGGT
AGTTAAACAAATTTATCCGGCTACCCCGGAGGTACTGGCGGAAATCCT
GGAACCAATCGTACCGGATCTGAAAGGCGTTCCGGGCGAGGCTGAGAA
AGCGGCGTCTGGCGTGGCGAAATGGCTGGCTGGTGCAGGCATCACTAT
GAAACTGAAAGACGCGGGTTTCCAGGCTGAAGATATCGCGCGTCTGAC
CGACCTGGCCTTCACCACTCCATCCCTGGAACTCCTGCTGTCTATGGC
ACCAGTAACTGCTGATCGTGAGCGTGTGAAAGCAATTTACCAGGACGC
ATTTTGAGCGGCCGC
SEQ ID NO: 11
pJB826_PaphII_pdcZp_adhAm
GCGGCCGCGGGGGGGGGGGGGAAAGCCACGTTGTGTCTCAAAATCTCT
GATGTTACATTGCACAAGATAAAAATATATCATCATGAACAATAAAAC
TGTCTGCTTACATAAACAGTAATACAAGGGGTCATATGTATACCGTTG
GTATGTACTTGGCAGAACGCCTAGCCCAGATCGGCCTGAAACACCACT
TTGCCGTGGCCGGTGACTACAACCTGGTGTTGCTTGATCAGCTCCTGC
TGAACAAAGACATGGAGCAGGTCTACTGCTGTAACGAACTTAACTGCG
GCTTTAGCGCCGAAGGTTACGCTCGTGCACGTGGTGCCGCCGCTGCCA
TCGTCACGTTCAGCGTAGGTGCTATCTCTGCAATGAACGCCATCGGTG
GCGCCTATGCAGAAAACCTGCCGGTCATCCTGATCTCTGGCTCACCGA
ACACCAATGACTACGGCACAGGCCACATCCTGCACCACACCATTGGTA
CTACTGACTATAACTATCAGCTGGAAATGGTAAAACACGTTACCTGCG
CACGTGAAAGCATCGTTTCTGCCGAAGAAGCACCGGCAAAAATCGACC
ACGTCATCCGTACGGCTCTACGTGAACGCAAACCGGCTTATCTGGAAA
TCGCATGCAACGTCGCTGGCGCTGAATGTGTTCGTCCGGGCCCGATCA
ATAGCCTGCTGCGTGAACTCGAAGTTGACCAGACCAGTGTCACTGCCG
CTGTAGATGCCGCCGTAGAATGGCTGCAGGACCGCCAGAACGTCGTCA
TGCTGGTCGGTAGCAAACTGCGTGCCGCTGCCGCTGAAAAACAGGCTG
TTGCCCTAGCGGACCGCCTGGGCTGCGCTGTCACGATCATGGCTGCCG
AAAAAGGCTTCTTCCCGGAAGATCATCCGAACTTCCGCGGCCTGTACT
GGGGTGAAGTCAGCTCCGAAGGTGCACAGGAACTGGTTGAAAACGCCG
ATGCCATCCTGTGTCTGGCACCGGTATTCAACGACTATGCTACCGTTG
GCTGGAACTCCTGGCCGAAAGGCGACAATGTCATGGTCATGGACACCG
ACCGCGTCACTTTCGCAGGACAGTCCTTCGAAGGTCTGTCATTGAGCA
CCTTCGCCGCAGCACTGGCTGAGAAAGCACCTTCTCGCCCGGCAACGA
CTCAAGGCACTCAAGCACCGGTACTGGGTATTGAGGCCGCAGAGCCCA
ATGCACCGCTGACCAATGACGAAATGACGCGTCAGATCCAGTCGCTGA
TCACTTCCGACACTACTCTGACAGCAGAAACAGGTGACTCTTGGTTCA
ACGCTTCTCGCATGCCGATTCCTGGCGGTGCTCGTGTCGAACTGGAAA
TGCAATGGGGTCATATCGGTTGGTCCGTACCTTCTGCATTCGGTAACG
CCGTTGGTTCTCCGGAGCGTCGCCACATCATGATGGTCGGTGATGGCT
CTTTCCAGCTGACTGCTCAAGAAGTTGCTCAGATGATCCGCTATGAAA
TCCCGGTCATCATCTTCCTGATCAACAACCGCGGTTACGTCATCGAAA
TCGCTATCCATGACGGCCCTTACAACTACATCAAAAACTGGAACTACG
CTGGCCTGATCGACGTCTTCAATGACGAAGATGGTCATGGCCTGGGTC
TGAAAGCTTCTACTGGTGCAGAACTAGAAGGCGCTATCAAGAAAGCAC
TCGACAATCGTCGCGGTCCGACGCTGATCGAATGTAACATCGCTCAGG
ACGACTGCACTGAAACCCTGATTGCTTGGGGTAAACGTGTAGCAGCTA
CCAACTCTCGCAAACCACAAGCGTAATTAACTCGAGTTGGATCCTATA
AGTAGGAGATAAACATATGTGGGAAACTAAGATTAATATCAACGAAGT
CCGTGAGATCCGCGCGAAAACCACCGTTTACTTTGGTGTTGGTGCTAT
CAAGAAAATTGATGATATCGCTCGCGAGTTCAAAGAAAAAGGTTACGA
TCGCATCATCGTGATCACCGGTAAAGGCGCTTACAAAGCGACCGGTGC
ATGGGAATACATCGTGCCTGCTCTGAACAAAAACCAGATTACGTATAT
CCATTATGATCAGGTGACCCCGAACCCGACCGTAGATCAGGTTGACGA
AGCGACCAAACAGGCCCGTGAATTTGGCGCTCGCGCAGTACTGGCTAT
TGGTGGCGGTTCCCCGATCGACGCAGCCAAATCTGTGGCGGTGCTGCT
GTCTTATCCGGACAAAAACGCTCGTCAGCTGTACCAGCTGGAGTTTAC
CCCGGTAAAAGCAGCGCCGATCATCGCCATCAACCTGACCCACGGTAC
GGGCACCGAAGCGGACCGCTTCGCGGTTGTATCTATCCCGGAGAAGGC
CTACAAACCGGCTATCGCTTACGATTGCATCTACCCGCTGTACTCTAT
TGACGACCCGGCTCTGATGGTTAAACTGCCGAGCGACCAGACGGCGTA
CGTTAGCGTGGATGCCCTGAACCATGTTGTTGAAGCTGCGACCTCCAA
AGTTGCATCTCCGTACACTATTATCCTGGCAAAAGAAACGGTCCGTCT
CATCGCACGCTACCTGCCTCAGGCCCTGTCTCACCCTGCAGACCTGAC
CGCGCGTTATTACCTCCTGTATGCCTCTCTGATCGCCGGTATTGCGTT
TGATAACGGCCTGCTGCATTTCACCCACGCACTGGAACACCCGCTGTC
TGCCGTGAAACCTGAACTGGCTCATGGCCTGGGTCTGGGTATGCTCCT
GCCTGCGGTAGTTAAACAAATTTATCCGGCTACCCCGGAGGTACTGGC
GGAAATCCTGGAACCAATCGTACCGGATCTGAAAGGCGTTCCGGGCGA
GGCTGAGAAAGCGGCGTCTGGCGTGGCGAAATGGCTGGCTGGTGCAGG
CATCACTATGAAACTGAAAGACGCGGGTTTCCAGGCTGAAGATATCGC
GCGTCTGACCGACCTGGCCTTCACCACTCCATCCCTGGAACTCCTGCT
GTCTATGGCACCAGTAACTGCTGATCGTGAGCGTGTGAAAGCAATTTA
CCAGGACGCATTTTGAGCGGCCGC

TABLE 4
Additional Informal Sequence Listing

SEQ ID: 3
TAACACCGTGCGTGTTGACTATTTTACCTCTGGCGGTGATAATGGTTG
CA
SEQ ID: 4
ATGAAAGGACCAATAATAATGACTAGAGAAGAAAGAATGAAGATTGTT
CATGAAATTAAGGAACGAATATTGGATAAATATGGGGATGATGTTAAG
GCAATTGGTGTTTATGGCTCTCTTGGTCGTCAGACTGATGGGCCCTAT
TCGGATATTGAGATGATGTGTGTTCTGTCAACAGAGGGAGTAGAGTTC
AGCTATGAATGGACAACCGGTGAGTGGAAGGCGGAAGTGAATTTTTAT
AGCGAAGAGATTCTACTAGATTATGCATCTCGGGTGGAACCGGATTGG
CCGCTTACACATGGTCGATTTTTCTCTATTTTGCCGATTTATGATCCA
GGTGGATACTTTGAGAAAGTGTACCAAACTGCTAAATCGGTAGAAGCC
CAAAAGTTCCACGATGCGATCTGTGCCCTTATCGTAGAAGAGCTGTTT
GAATATGCAGGCAAATGGCGTAATATTCGTGTGCAAGGACCGACAACA
TTTCTACCATCCTTGACTGTACAGGTGGCAATGGCAGGTGCCATGTTG
ATTGGTCTGCATCATCGCATCTGTTATACGACGAGCGCTTCGGTCTTA
ACTGAAGCAGTTAAGCAACCAGATCTTCCTCCAGGTTATGTCCAACTG
TGCCAGCTCGTAATGTCTGGTCAACTTTCCGACCCTGAGAAACTTCTG
GAATCGCTAGAGAATTTCTGGAATGGGGTTCAGGAGTGGGCGGAACGA
CACGGATATATAGTGGATGTGTCAAAACGCATACCATTTTGA

We claim:

1. A method for producing a carbon-based product of interest, comprising:
a. preparing a heterologous DNA sequence operably linked to an expression vector;
b. transforming a thermophilic cyanobacterium host with said vector; and
c. culturing said host.

2. A method for producing a fuel composition, comprising:
a. preparing a heterologous DNA sequence operably linked to an expression vector;
b. transforming a thermophilic cyanobacterium host with said vector; and
c. culturing said host.

3. The method of claim 1 wherein said carbon-based product of interest is selected from the group consisting of: ethyl ester, methyl ester, sucrose, alcohol, ethanol, propanol, isopropanol, butanol, fatty alcohols, fatty acid ester, wax ester, hydrocarbons, n-alkanes, propane, octane, diesel, JP8, polymers, terephthalate, polyol, 1,3-propanediol, 1,4-butanediol, PHA, PHB, acrylate, adipic acid, ε-caprolactone, isoprene, caprolactam, rubber, lactate, DHA, 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid, carotenoid, isoprenoid, itaconic acid, limonene, pharmaceutical or pharmaceutical intermediates, erythromycin 7-ADCA/cephalosporin, polyketides, statin, paclitaxel, docetaxel, terpene, peptide, steroid, and an omega fatty acid.

4. The method of claim 1 wherein said expression vector comprises an isolated or recombinant polynucleotide comprising or consisting of a nucleic acid sequence selected from the group consisting of:
a. any one of the sequences from Table 3;
b. a nucleic acid sequence at least 80%, at least 85%, at least 90%, at least 95%, at least 98%, at least 99% or at least 99.9% identical to any one of the sequences from Table 3; and
c. a nucleic acid sequence that hybridizes under stringent conditions to any one of the sequences in Table 3.

5. The method of claim 1 wherein said thermophilic cyanobacterium is Thermosynechococcus elongatus BP-1.

6. The method of claim 1 wherein transforming said thermophilic cyanobacterium host comprises integrating at least a portion of said vector in a chromosome of said thermophilic cyanobacterium.

7. The method of claim 1 further comprising isolating said carbon-based product of interest from said host cell or a culture medium.

8. The method of claim 2 further comprising isolating said fuel composition from said host cell or a culture medium.

9. The method of claim 1 wherein said carbon-based product of interest is an alcohol.

10. The method of claim 1 wherein said carbon-based product of interest is ethanol.

11. The method of claim 1 wherein said carbon-based product of interest is ethanol, and wherein said cyanobacterium produces at least 1000, at least 5000, at least 10,000, at least 12,000, or at least 15,000 mgs ethanol per liter of culture medium.

12. The method of claim 1 wherein said carbon-based product of interest is ethanol, and wherein said cyanobacterium produces between 1000 and 20,000 mgs ethanol per liter of culture medium.

13. The method of claim 1 wherein said carbon-based product of interest is ethanol, and wherein said cyanobacterium produces between 10,000 and 20,000, between 12,000 and 18,000, or between 13,000 and 16,000 mgs ethanol per liter of culture medium.

14. The method of claim 1 wherein said carbon-based product of interest is ethanol, and wherein said cyanobacterium further produces acetaldehyde, and wherein the ratio of ethanol to acetaldehyde is at least 500, at least 2000, at least 4000, at least 4500, at least 5000, at least 10,000, or between 4000 and 15,000, or between 500 and 3,000.

15. A modified Thermosynechococcus cell comprising a recombinant marker gene and a λ phage cI promoter wherein said marker gene is operably linked to said promoter.

16. The cell of claim 15 wherein said marker gene confers antibiotic resistance to said cell.

17. The cell of claim 15 wherein said marker gene confers resistance to kanamycin to said cell.

18. The cell of claim 15 wherein said marker gene is htk.

19. An isolated or recombinant polynucleotide comprising or consisting of a nucleic acid sequence selected from the group consisting of:
a. any one of the sequences from Table 3;
b. a nucleic acid sequence at least 80%, at least 85%, at least 90%, at least 95%, at least 98%, at least 99% or at least 99.9% identical to any one of the sequences from Table 3; and
c. a nucleic acid sequence that hybridizes under stringent conditions to any one of the sequences in Table 3.

20. A modified Thermosynechococcus cell comprising an alcohol dehydrogenase gene and a pyruvate decarboxylase gene.

21. The cell of claim 20 wherein at least one of said alcohol dehydrogenase gene and said pyruvate decarboxylase gene is recombinant.

22. The cell of claim 20 further comprising at least one promoter.

23. The cell of claim 22 wherein said at least one promoter is selected from the group consisting of tef, tac, trp, tet, trp-tet, lpp, lac, lpp-lac, lacIq, T7, T5, T3, gal, trc, ara, SP6, amyE, phage SP02, Pcpcb, PaphII, PtRNA_{Glu}, λ phage cI λ-p_{R} and λ-p_{L}.

24. The cell of claim 22 wherein said at least one promoter is PaphII.

25. The cell of claim 20 comprising SEQ ID NO:11.

26. The cell of claim 20 wherein said genes are divergently oriented.

27. The cell of claim 20 further comprising a first promoter operably linked to said alcohol dehydrogenase gene and a second promoter operably linked to said pyruvate decarboxylase gene.

28. The cell of claim 27 where said first promoter and said second promoter are each independently selected from the group consisting of tef, tac, trp, tet, trp-tet, lpp, lac, lpp-lac, lacIq, T7, T5, T3, gal, trc, ara, SP6, amyE, phage SP02, Pcpcb, PaphII, PtRNA_{Glu}, λ phage cI λ-p_{R} and λ-p_{L}

29. The cell of claim 27 wherein at least one of said first promoter and said second promoter is λ phage cI.

30. The cell of claim 27 wherein said first promoter is λ phage cI and said second promoter is PEM7.

31. The cell of claim 27 wherein said first promoter is PEM7 and said second promoter is λ phage cI.

32. The cell of claim 27 wherein said first promoter is λ phage cI and said second promoter is PtRNA_{Glu}.

33. The cell of claim 27 wherein said first promoter is PtRNA_{Glu} and said second promoter is λ phage cI.

34. The cell of claim 27 wherein said first promoter is PaphII and said second promoter is λ phage cI.

35. The cell of claim 27 wherein said first promoter is Pcpcb and said second promoter is λ phage cI.

36. The cell of claim 20 comprising any one of SEQ ID NO:5, SEQ ID NO:6, SEQ ID NO:7, SEQ ID NO:8, SEQ ID NO:9 or SEQ ID NO:10.

37. A method of producing a carbon-based product of interest comprising culturing the cell of claim 15 wherein said cell produces said carbon-based product of interest.

38. The method of claim 37 wherein said carbon-based product of interest is selected from the group consisting of: ethyl ester, methyl ester, sucrose, alcohol, ethanol, propanol, isopropanol, butanol, fatty alcohols, fatty acid ester, wax ester, hydrocarbons, n-alkanes, propane, octane, diesel, JP8, polymers, terephthalate, polyol, 1,3-propanediol, 1,4-butanediol, PHA, PHB, acrylate, adipic acid, ε-caprolactone, isoprene, caprolactam, rubber, lactate, DHA, 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid, carotenoid, isoprenoid, itaconic acid, limonene, pharmaceutical or pharmaceutical intermediates, erythromycin 7-ADCA/cephalosporin, polyketides, statin, paclitaxel, docetaxel, terpene, peptide, steroid, and an omega fatty acid.

39. The method of claim 37 wherein the carbon-based product of interest is an alcohol.

40. The method of claim 37 wherein the carbon-based product of interest is ethanol.

41. The method of claim 37 wherein said carbon-based product of interest is ethanol, and wherein said cyanobacterium produces at least 1000, at least 5000, at least 10,000, at least 12,000, or at least 15,000 mgs ethanol per liter of culture medium.

42. The method of claim 37 wherein said carbon-based product of interest is ethanol, and wherein said cyanobacterium produces between 1000 and 20,000 mgs ethanol per liter of culture medium.

43. The method of claim 37 wherein said carbon-based product of interest is ethanol, and wherein said cyanobacterium produces between 10,000 and 20,000, between 12,000 and 18,000, or between 13,000 and 16,000 mgs ethanol per liter of culture medium.

44. The method of claim 37 wherein said carbon-based product of interest is ethanol, and wherein said cyanobacterium further produces acetaldehyde, and wherein the ratio of ethanol to acetaldehyde is at least 500, at least 2000, at least 4000, at least 4500, at least 5000, at least 10,000, or between 4000 and 15,000, or between 500 and 3,000.

45. A method for engineering a thermophilic cyanobacterium comprising transforming said thermophilic cyanobacterium with a heterologous DNA sequence operably linked to an expression vector.

46. The method of claim 45 wherein said expression vector comprises an isolated or recombinant polynucleotide comprising or consisting of a nucleic acid sequence selected from the group consisting of:
a. any one of the sequences from Table 3;
b. a nucleic acid sequence at least 80%, at least 85%, at least 90%, at least 95%, at least 98%, at least 99% or at least 99.9% identical to any one of the sequences from Table 3; and
c. a nucleic acid sequence that hybridizes under stringent conditions to any one of the sequences in Table 3.

47. The method of claim 45 wherein said thermophilic cyanobacterium is Thermosynechococcus elongatus BP-1.

48. The method of claim 45 wherein transforming said thermophilic cyanobacterium host comprises integrating at least a portion of said vector in a chromosome of said thermophilic cyanobacterium.
